(19) United States
(12) Patent Application Publication
(10) Pub. No.: US 2026/0015352 A1
(43) Pub. Date: Jan. 15, 2026

(54) METHYLTRANSFERASE INHIBITOR AND USE THEREOF

(71) Applicant: CYTOSINLAB THERAPEUTICS CO., LTD., Shanghai (CN)

(72) Inventors: Xingnian FU, Shanghai (CN); Yuan MI, Shanghai (CN); Haiping WU, Shanghai (CN); Meng WANG, Shanghai (CN); Long WANG, Shanghai (CN); Hui SHI, Shanghai (CN); Yang HAN, Shanghai (CN)

(21) Appl. No.: 18/993,786

(22) PCT Filed: Jul. 11, 2023
(86) PCT No.: PCT/CN2023/106840

§ 371 Date: Jan. 13, 2025

(30) Foreign Application Priority Data
Jul. 12, 2022 (CN) 202210822809.7
Dec. 30, 2022 (CN) 202211729476.X

(51) Int. Cl. C07D 471/04; A61K 31/4375; A61K 31/444; A61K 31/506; A61K 31/519; A61K 31/5377; A61K 31/541; C07D 405/14; C07D 409/14; C07D 487/04; C07D 519/00

(52) U.S. Cl. CPC C07D 471/04; A61K 31/4375; A61K 31/444; A61K 31/506; A61K 31/519; A61K 31/5377; A61K 31/541; C07D 405/14; C07D 409/14; C07D 487/04; C07D 519/00

(57) ABSTRACT

A class of compounds having methyltransferase inhibitory activity. A compound has a structure as represented by formula I, and can be used for treating and preventing diseases related to a PRC2 complex or a constituent monomer thereof.

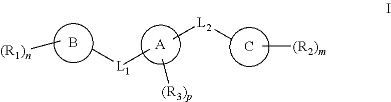

### TECHNICAL FIELD

The present invention relates to the field of pharmaceutical compounds, specifically the present invention provides a class of compounds for inhibiting PRC2 complexes, monomers, or combinations thereof, and their use in drugs or drug combinations.

### BACKGROUND

Site-specific lysine methylation on histones is one of the important epigenetic mechanisms that control and mediate many fundamental biological processes.

The epigenome behaves differently in transcriptionally active and silenced regions, and the polycomb repressive complex 2(PRC2) plays a crucial role in gene silencing by selectively occupying chromatin transcriptional silencing sites. PRC2 consists of four core components and several cofactors that regulate its activity: the enhancer of zeste 1 or 2 (EZH1/2), embryonic ectodermal development (EED), the inhibitor of zeste 12 (SUZ12), and retinoblastoma associated proteins 46 and 48 (RBAP46/RBAP48). EZH1/2 is a methyltransferase catalytic subunit responsible for the mono-, di-, and tri-methylation of lysine residues at position 27 of histone H3 (H3K27me1/2/3). EED enhances the enzymatic activity of PRC2 by binding to H3K27me3. SUZ12 interacts with all other subunits and contributes to the stability of the complex. RBAP46/RBAP48 (also known as RBBP4/7) can recognize histones H3 and H4 and may regulate the substrate specificity of PRC2. PRC2 is currently the only known methyltransferase of H3K27, and mediates trimethylation of H3K27 (H3K27me3), which ultimately leads to gene silencing at specific site by altering chromatin structure (chromatin compaction).

In human diseases, including tumors, PRC2 protein often undergoes mutations or dysregulation. Studies have found that PRC2 component is overexpressed in many solid tumors (including but not limited to ovarian cancer, breast cancer, prostate cancer, endometrial cancer, melanoma, bladder cancer, lung cancer, and liver cancer). In addition, specific missense mutations can enhance the catalytic activity of PRC2 in patients with hematologic malignancies such as DLBCL (diffuse large B-cell lymphoma), and FL (follicular lymphoma). At the same time, some in vivo and in vitro results also show that the proliferation and metastasis of many tumor cell lines depend on the activity of PRC2. Therefore, the inhibition of EZH2 or other core components of PRC2 by gene knockdown or knockout or small molecule drugs can inhibit the proliferation of many types of tumor cell lines (such as leukemia, lymphoma, prostate cancer, breast cancer, lung cancer and kidney cancer). Current preclinical studies have shown that secondary mutations in EZH2 can lead to acquired resistance, and its homologous EZH1 also has methyltransferase activity, both of which can result in limited EZH2 inhibitor activity. Meanwhile, the EZH2 subunit itself is inactive and must assemble with SUZ12 and EED to form a PRC2 protein complex in order to produce methyltransferase activity. Therefore, the development of novel inhibitors targeting the entire PRC2 protein complex has attracted many attentions.

In addition, IFN-γ plays a crucial role in the pathogenesis of many autoimmune diseases, and the relative deficiency of IFN-γ is a characteristic of some chronic infections and tumor tolerance in humans. The inhibition of EZH2 significantly increases the expression of IFN-γ in immature CD4+ T cells, which may promote IFN-γ-dependent anti-tumor immunity, but the promotion of IFN-γ expression by EZH2 inhibition relies on other components of PRC2.

Therefore, PRC2 provides a pharmacological target for solid tumors and some hematologic malignancies. Targeted inhibition of EZH2 methyltransferase activity has been proven to be a successful cancer treatment strategy and may also play an important role in tumor immunity.

### SUMMARY OF THE INVENTION

The purpose of the present invention is to provide a small molecule compound for inhibiting PRC2 protein complex or one or combinations of more proteins that constitute the complex.

In the first aspect of the present invention, provided is a compound of formula I, or a pharmaceutically acceptable salt thereof or a deuterated compound, a racemic mixture, or an optical monomer thereof:

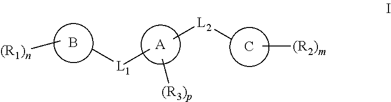

wherein, ring A is selected from the group consisting of: 5-10 membered bridged ring (including carbocycle and heterocycle), 6-10 membered aromatic ring, and 5-10 membered heteroaromatic ring;
ring B is selected from the group consisting of: 6-10 membered aromatic ring, and 5-14 membered heteroaromatic ring;
ring C is selected from the group consisting of: 5-10 membered aromatic ring or partially saturated aromatic ring, 5-10 membered heteroaromatic ring or partially saturated heteroaromatic ring, 5-10 membered saturated or partially unsaturated carbocycle (including fused and bridged rings), 5-10 membered saturated or partially unsaturated heterocycle (including fused and bridged rings);
L₁ and L₂ are each independently -(L)ₚ-, and each L is independently selected from the group consisting of: chemical bond or none, or —O—, —CHR—, —CHR—NH—, carbonyl, S, —NR—, —NHC(O)—, —NHS(O)₂—, —NHC(O)NH—, —NHC(S)NH—, —COO—, —O—S(O)₂—, —CHR—NR—, —C(R)₂NR—, and —C(R)₂—; wherein, each R is independently selected from the group consisting of: H, substituted or unsubstituted C₁₋₄ alkyl, substituted or unsubstituted C₃₋₄ cycloalkyl, and substituted or unsubstituted 3-4 membered heterocyclyl;
n is selected from the group consisting of: 0, 1, 2, 3, 4 and 5;
m is selected from the group consisting of: 0, 1, 2, 3 and 4;
p is selected from the group consisting of: 0, 1 and 2;
each R₁, R₂, and R₃ is independently selected from the group consisting of: H, halogen, cyano, amino, nitro, hydroxyl, thiol, aldehyde group, carboxyl, sulfonyl, substituted or unsubstituted C₁-C₆ alkyl, substituted or unsubstituted C₁-C₆ alkoxy, substituted or unsubstituted C₁-C₆alkylamino, substituted or unsubstituted C₁-C₆ alkylamide, substituted or unsubstituted C₁-C₆ alkyl-C(O)O, substituted or unsubstituted C₁-C₆ alkyl-OC(O), substituted or unsubstituted amide, substituted or unsubstituted amino (NH₂), substituted or unsubstituted C₂-C₆ alkenyl, substituted or unsubstituted C₂-C₆ alkynyl, substituted or unsubstituted C₃-C₆ carbocycle (including saturated or partially unsaturated situation), and substituted or unsubstituted 3-6 membered heterocycle (including saturated or partially unsaturated situation), and —X—Z—Y—R₅;
wherein, X and Y are each independently selected from the group consisting of: chemical bond, —O—, —C(R₅)₂—, —S—, and —NR₅—;
Z is selected from the group consisting of: C(O), NH, CH═CH, —C(R₅)₂—, S(O), and S(O)₂;
each R₅ is independently selected from the group consisting of: H, halogen, cyano, amino, nitro, hydroxyl, thiol, aldehyde group, carboxyl, sulfonyl, substituted or unsubstituted C₁-C₆ alkyl, substituted or unsubstituted C₃-C₆ carbocycle (including saturated or partially unsaturated situation), substituted or unsubstituted 3-6 membered heterocycle, substituted or unsubstituted C₆₋₁₀ aromatic ring, substituted or unsubstituted 5 to 12 membered heteroaromatic ring, substituted or unsubstituted C₁-C₆ alkyl-C(O)O, or substituted or unsubstituted C₁-C₆ alkyl-OC(O), and substituted or unsubstituted 5 to 9 membered heterospiro ring;
or two R₁, R₂, or R₃ located at two adjacent ring atoms together form a fused ring structure selected from the group consisting of: substituted or unsubstituted C₆₋₁₀ aromatic ring, substituted or unsubstituted 5 to 12 membered heteroaromatic ring, substituted or unsubstituted C₃-C₈ carbocycle (including saturated or partially unsaturated situation), substituted or unsubstituted 3 to 8 membered heterocycle (including saturated or partially unsaturated situation); or two adjacent R₁, R₂, or R₃ located at the same ring carbon atom together with the attached ring form a 3 to 8 membered saturated or partially unsaturated spiro ring structure, or a 3 to 8 membered saturated or partially unsaturated heterospiro ring structure (the 3-8 membered ring referred herein is the ring formed by the substituents, which does not include the attached ring);
wherein, the ring skeleton of each heterocycle mentioned above may contain 1-3 heteroatoms selected from boron, oxygen, sulfur, phosphorus, and nitrogen; specifically, when the atom is boron, sulfur, phosphorus, or nitrogen, the ring skeleton atom can be oxidized, such as S(O) or S(O)₂;
unless otherwise specified, in the above formulas, the “substituted” refers to the corresponding group is substituted by one or more substituents selected from the group consisting of: deuterium, tritium, halogen, oxo, =NH, =N(C₁₋₈ alkyl), hydroxy, carboxy, thiol, benzyl, C₁-C₁₂ alkoxycarbonyl, C₁-C₆ aldehyde group, amino, C₁-C₆ amide, nitro, cyano, unsubstituted or halogenated C₁-C₆ alkyl, C₁-C₆ alkyl-CN, unsubstituted or halogenated C₃-C₈ cycloalkyl, C₂-C₁₀ alkenyl, C₂-C₁₀ alkynyl, C₁-C₆ alkoxy, C₁-C₆ alkyl-amino, C₆-C₁₀ aryl, 5- or 6-membered heteroaryl, 3-8 membered non-aromatic heterocyclyl, —O—(C₆-C₁₀ aryl), —O-(5- or 6-membered heteroaryl), C₁-C₁₂ alkylamino carbonyl, unsubstituted or halogenated C₂-C₁₀ acyl, sulfonyl (—SO₂—OH), phosphoryl (—PO₃—OH), unsubstituted or halogenated C₁-C₄ alkyl-S(O)₂—, unsubstituted or halogenated C₁-C₄ alkyl-SO—, and unsubstituted or halogenated C₁-C₄ alkylamino-S(O)₂—;
wherein, each chiral atom in the molecule can be in R configuration, S configuration, or a combination thereof.

In another preferred embodiment, ring B is a 6 to 10 membered heteroaromatic ring, and R₁ is selected from the group consisting of: H, halogen, cyano, amino, nitro, hydroxyl, thiol, aldehyde group, carboxyl, sulfonyl, substituted or unsubstituted C₁-C₆ alkyl, substituted or unsubstituted C₁-C₆ alkoxy, and substituted or unsubstituted C₁-C₆ alkylamino; or two R₁ attached to adjacent ring atoms together form a cyclic structure selected from the group consisting of: substituted or unsubstituted C₃-C₆ carbocycle (including saturated or partially unsaturated situation), and substituted or unsubstituted 3-6 membered heterocycle (including saturated and partially unsaturated situation).

In another preferred embodiment, ring B has a structure selected from the group consisting of (wherein the connecting site can be located on any ring atom):

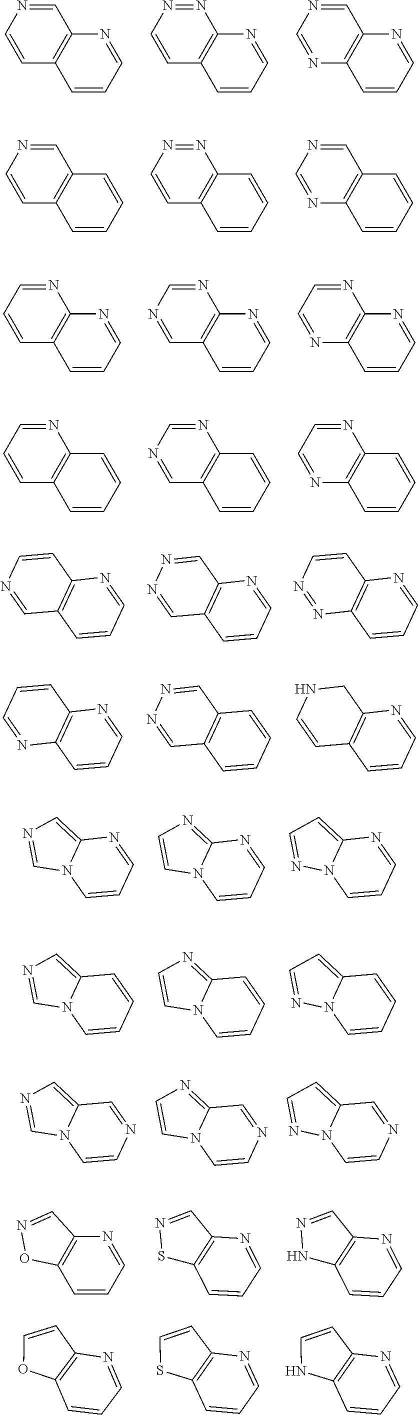

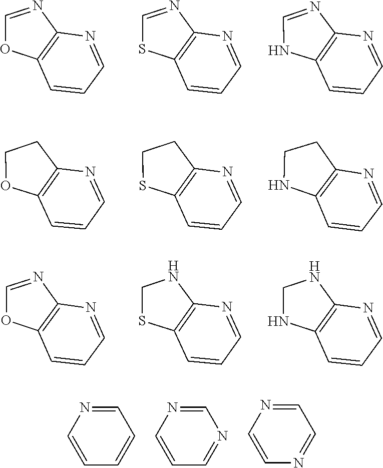

In another preferred embodiment, ring A has a structure selected from the group consisting of (wherein the connecting site can be located on any ring atom):

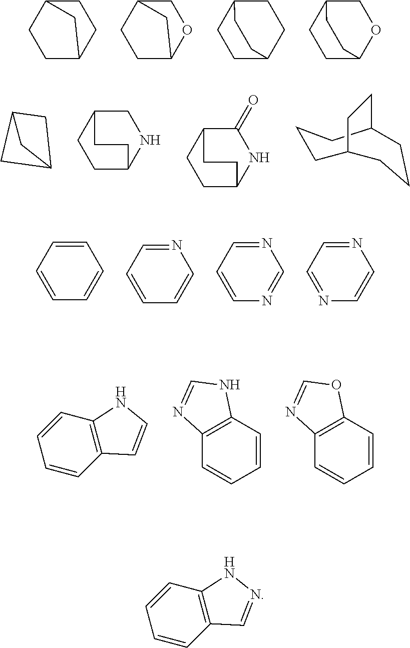

In another preferred embodiment, R₂ has a structure as shown in the following:

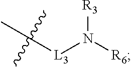

wherein, L₃ is selected from the group consisting of: —C(R₅)₂—C(O)— and —C(R₅)₂—;
R₃ is selected from the group consisting of: H, substituted or unsubstituted C₁-C₆ alkyl;
R₆ is selected from the group consisting of: substituted or unsubstituted C₁-C₆ alkyl, substituted or unsubstituted C₃-C₆carbocycle(including saturated or partially unsaturated situation), substituted or unsubstituted 3-6 membered heterocycle; the ring skeleton of the heterocycle may contain 1-3 heteroatoms selected from oxygen and sulfur; and the ring skeleton atoms can be oxidized;
the “substituted” refers to the hydrogen atoms on the corresponding group is substituted by one or more substituents selected from the group consisting of: deuterium, tritium, halogen, oxo, hydroxyl, carboxyl, thiol, benzyl, cyano, unsubstituted or halogenated C₁-C₆ alkyl, unsubstituted or halogenated C₃-C₈ cycloalkyl, C₂-C₁₀ alkenyl, C₁-C₆ alkoxy, C₁-C₆ alkyl-amino, C₆-C₁₀ aryl, 5- or 6-membered heteroaryl, and 3-8 membered non-aromatic heterocyclyl.

In another preferred embodiment, R₂ is —CHR—C(O)NH—R₆, wherein R is H, or substituted or unsubstituted C₁₋₄ alkyl, substituted or unsubstituted C₁-C₆ alkoxy, hydroxyl, substituted or unsubstituted amino; R₆ is selected from the group consisting of: substituted or unsubstituted C₃-C₆ carbocycle (including saturated or partially unsaturated situation), and substituted or unsubstituted 3-to-6 membered heterocycle (including saturated or partially unsaturated situation).

In another preferred embodiment, the compound has a structure as shown in formula II below:

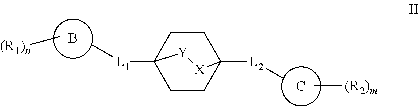

wherein, X and Y are independently selected from the group consisting of: O, NR₃, C(R₃)₂, and —C(═O)—;
or the compound has a structure as shown in formula III below:

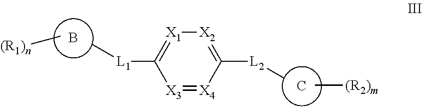

wherein, X₁, X₂, X₃, or X₄ is each independently selected from the group consisting of: N and CR₃;
or the compound has a structure as shown in Formula IV below:

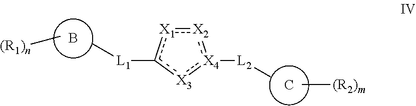

wherein, X₁, X₂, X₃, or X₄ is each independently selected from the group consisting of: O, S, N, NR₃ and CR₃;
[symbol] represents single bond or double bond;
the remaining groups are as described in the first aspect of the present invention.

In another preferred embodiment, L₁ is selected from the group consisting of: chemical bond, —O—, —CHR—, carbonyl, S, and —NH—; L₂ is selected from the group consisting of: chemical bond, —CHR—NH—, —CHR—O—, —CHR—S—, and —(CHR)₂—.

In the second aspect of the present invention, provided is a pharmaceutical composition comprising a therapeutically effective amount of one or more of the compound according to the first aspect of the present invention, a pharmaceutically acceptable salt thereof, a racemate, an optical isomer, a stereoisomer, and a tautomer thereof, and one or more pharmaceutically acceptable carriers, excipients, adjuvants, excipients, and/or diluents.

In the third aspect of the present invention, provided is a use of the compound according to the first aspect of the present invention, a racemate, an optical isomer monomer and mixtures thereof, or the pharmaceutically acceptable salt thereof in the preparation of a drug for the treatment or prevention of diseases associated with mutations, and overexpression of PRC2 complexes, monomers, or combinations thereof, or H3K27 methylation level dysregulation.

In another preferred embodiment, the disease is selected from the group consisting of: tumors and autoimmune diseases.

In another preferred embodiment, the disease is selected from the group consisting of: lymphoma, malignant blood disease, sarcoma, prostate cancer, breast cancer, kidney cancer, urothelial cancer, gastric cancer, ovarian cancer, endometrial cancer, cervical cancer, lung cancer, liver cancer, pancreatic cancer, colon cancer, head and neck cancer, brain tumor, melanoma, mesothelioma, gastrointestinal stromal tumor, psoriasis, and lupus erythematosus.

In another preferred embodiment, the disease is selected from the group consisting of: non-Hodgkin lymphoma, follicular lymphoma, mantle cell lymphoma, peripheral T-cell lymphoma, Burkitt lymphoma, Hodgkin lymphoma, chronic lymphocytic leukemia, acute and chronic myeloid leukemia, acute and chronic lymphocytic leukemia, multiple myeloma, myelodysplastic syndrome, epithelioid sarcoma, rhabdomyosarcoma, liposarcoma, prostate cancer, breast cancer, kidney cancer, bladder cancer, upper urinary tract epithelial cancer, gastric cancer, ovarian cancer, endometrial cancer, cervical cancer, lung cancer, liver cancer, pancreatic cancer, colon cancer, head and neck cancer, medulloblastoma, glioma, schwannoma, melanoma, mesothelioma, gastrointestinal stromal tumor, psoriasis and lupus erythematosus.

It should be understood that, within the scope of the present invention, each of the above technical features of the present invention and each of the technical features specifically described in the following (such as the examples) can be combined with each other to constitute a new or preferred technical solution. Due to space limitations, It will not be repeated herein.

### EMBODIMENTS FOR CARRYING OUT THE INVENTION

After long and intensive research, the inventors unexpectedly discovered a class of compound with H3K27 methylation level regulatory effects for the first time. The present invention is completed on this basis.

### Term

As used herein, halogen refers to F, Cl, Br or I.

As used herein, unless otherwise specified, the terms used have a general meaning known to those skilled in the art. As used herein, unless otherwise specified, all chemical formulas are intended to encompass any possible optical or geometric isomers (such as R-type, S-type or racemate, or cis-trans isomers of olefins, etc.).

As used herein, the term “C1-C6 alkyl” refers to a linear or branched alkyl having 1 to 6 carbon atoms, including but not limited to methyl, ethyl, propyl, isopropyl, butyl, isobutyl, sec-butyl, tert-butyl, pentyl and hexyl and the like; preferably ethyl, propyl, isopropyl, butyl, isobutyl, sec-butyl and tert-butyl.

As used herein, the term “C1-C6 alkoxyl” refers to a linear or branched alkoxy having 1 to 6 carbon atoms, including but not limited to methoxy, ethoxy, propoxy, isopropoxy and butoxy and the like.

As used herein, the term “C2-C6 alkenyl” refers to a linear or branched alkenyl having 2 to 6 carbon atoms and containing a double bond, including but not limited to vinyl, propenyl, butenyl, isobutenyl, pentenyl, hexenyl, and the like.

As used herein, the term “C2-C6 alkynyl” refers to a linear or branched alkynyl having 2 to 6 carbon atoms and containing a triple bond, including but not limited to ethynyl, propynyl, butynyl, isobutynyl, pentynyl, hexynyl, and the like.

As used herein, the term “C3-C10 cycloalkyl” refers to a cyclic alkyl having 3 to 10 carbon atoms on the ring, including but not limited to cyclopropyl, cyclobutyl, cyclopentyl, cyclohexyl, cycloheptyl, cyclooctyl, cyclodecyl and the like. The terms “C3-C8 cycloalkyl”, “C3-C7 cycloalkyl”, and “C3-C6 cycloalkyl” have similar meanings.

As used herein, the term “C3-C10 cycloalkenyl” refers to a cyclic alkenyl having 3 to 10 carbon atoms on the ring, including but not limited to cyclopropenyl, cyclobutenyl, cyclopentenyl, cyclohexenyl, cycloheptenyl, cyclooctenyl, cyclodecenyl and the like. The term “C3-C7 cycloalkenyl” has a similar meaning.

As used herein, the term “C1-C12 alkoxycarbonyl” refers to an alkoxycarbonyl having 1 to 12 carbon atoms on the alkyl chain, including but not limited to methoxycarbonyl, ethoxycarbonyl, propoxycarbonyl, isopropoxycarbonyl, tert butoxycarbonyl, benzyloxycarbonyl, and the like.

As used herein, the term “C1-C12 alkylaminocarbonyl” refers to an alkylaminocarbonyl having 1 to 12 carbon atoms on the alkyl chain, including but not limited to methylamino carbonyl, ethylamino carbonyl, propylamino carbonyl, isopropylamino carbonyl, tert butylamino carbonyl, benzylamino carbonyl, dimethylamino carbonyl and the like.

As used herein, the terms “aromatic ring” or “aryl” have the same meaning, preferably “aryl” is “C6-C12 aryl” or “C6-C10 aryl”. The term “C6-C12 aryl” refers to an aromatic ring group having 6 to 12 carbon atoms without any heteroatoms on the ring, such as phenyl, naphthyl and the like. The term “C6-C10 aryl” has a similar meaning.

As used herein, the terms “heteroaromatic ring” or “heteroaryl” have the same meaning, referring to heteroaromatic groups containing one to more heteroatoms. The heteroatoms referred to herein include oxygen, sulfur, and nitrogen. For example, furyl, thienyl, pyridinyl, pyrazolyl, pyrrolyl, N-alkyl pyrrolyl, pyrimidinyl, pyrazinyl, imidazolyl, tetrazolyl, and the like. Heteroaryl may be fused onto an aromatic, heterocyclic, or cycloalkyl ring, wherein the ring connected to the parent structure via a heteroaryl ring. Heteroaryl may be optionally substituted or unsubstituted.

As used herein, the term “3-12 membered heterocyclyl” refers to a saturated or unsaturated 3-12 membered cyclic group containing 1-3 heteroatoms selected from oxygen, sulfur, and nitrogen on the ring, such as dioxocyclopentyl and the like. The term “3-7-membered heterocyclyl” has a similar definition.

As used herein, the term “substituted” indicates that one or more hydrogen atoms on a specific group are substituted by specific substituents. The specific substituents are the substituents described above, or the substituents appeared in each example. Unless otherwise specified, a substituted group may have a substituent selected from a specific group at any substitutable site of that group, and the substituents may be the same or different in each position. A cyclic substituent, such as a heterocyclicalky, can be linked to another ring, such as a cycloalkyl, thereby forming a spiro-dicyclic ring system, where the two rings share a common carbon atom. It should be understood by those skilled in the art that the combinations of substituents contemplated by the present invention are those that are stable or chemically achievable. The substituents, such as (but not limited to): C1-8 alkyl, C2-8 alkenyl, C2-8 alkynyl, C3-8 cycloalkyl, 3- to 12-membered heterocyclyl, aryl, heteroaryl, halogen, hydroxyl, carboxyl (—COOH), C1-8 aldehyde group, C2-10 acyl, C2-10 ester group, C1-C12 alkoxycarbonyl, amino, alkoxyl, C1-10 sulfonyl, etc.

### H3K27 Methylation Level Regulator Compound

The present invention provides a class of compounds with H3K27 methylation level moderation effect:

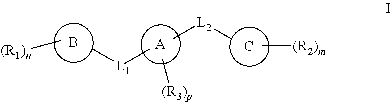

wherein, ring A is selected from the group consisting of: 5-to-10 membered bridged ring (including carbocycle and heterocycle), 6-to-10 membered aromatic ring, and 5-to-10 membered heteroaromatic ring (preferably benzene ring or 5-to-6 membered heteroaromatic ring);
ring B is selected from the group consisting of: 6-to-10 membered aromatic ring, 5-to-14 membered heteroaromatic ring (preferably 8-to-14 membered heteroaromatic ring), and partially saturated 5-to-12 membered heterocycle (preferably 8-to-14 membered heteroaromatic ring);
ring C is selected from the group consisting of: 5-to-10 membered aromatic ring or partially saturated aromatic ring, 5-to-10 membered heteroaromatic ring or partially saturated heteroaromatic ring, 5-to-10 membered saturated or partially unsaturated carbocycle (including fused and bridged rings), and 5-to-10 membered saturated or partially unsaturated heterocycle (including fused and bridged rings);
L₁ and L₂ are each independently -(L)ₚ-, and each L is independently selected from the group consisting of: chemical bond, none, —O—, —CHR—, —CHR—NH—, carbonyl, S, —NH—, —NHC(O)—, —NHS(O)₂—, —NHC(O)NH—, —NHC(S)NH—, —COO—, —O—S(O)₂—, —NR—, —C(R)₂NH—, and —C(R)₂—NR—; wherein, each R is selected from the group consisting of: H, substituted or unsubstituted C₁₋₄ alkyl or cycloalkyl, and substituted or unsubstituted 3-to-4 membered heterocyclyl;
n is selected from the group consisting of: 0, 1, 2, 3, 4, and 5;
m is selected from the group consisting of: 0, 1, 2, 3, and 4;
p is selected from the group consisting of: 0, 1, and 2;
each R₁, R₂, and R₃ is independently selected from the group consisting of: H, halogen, cyano, amino, nitro, hydroxyl, thiol, aldehyde group, carboxyl, sulfonyl, substituted or unsubstituted C₁-C₆ alkyl, substituted or unsubstituted C₁-C₆ alkoxy, substituted or unsubstituted C₁-C₆alkylamino, substituted or unsubstituted C₁-C₆ alkylamide or ester group, substituted or unsubstituted amide, substituted or unsubstituted amino (NH₂), substituted or unsubstituted C₂-C₆ alkenyl, substituted or unsubstituted C₂-C₆ alkynyl, substituted or unsubstituted C₃-C₆ carbocycle (including saturated or partially unsaturated situation), and substituted or unsubstituted 3-to-6 membered heterocycle (including saturated or partially unsaturated situation), or —X—Z—Y—R₅; wherein, X and Y are each independently selected from the group consisting of: chemical bond, —O—, —C(R₅)₂—, —S—, and —NR₅—; R₅ is selected from the group consisting of: H, halogen, cyano, amino, nitro, hydroxyl, thiol, aldehyde group, carboxyl, sulfonyl, substituted or unsubstituted C₁-C₆ alkyl, substituted or unsubstituted C₃-C₆ carbocycle (including saturated or partially unsaturated situation), and substituted or unsubstituted 3-6 membered heterocycle; or two R₁, R₂, or R₃ located at two adjacent ring atoms together form a fused ring structure selected from the group consisting of: substituted or unsubstituted C₆₋₁₀ aromatic ring, substituted or unsubstituted 5 to 12 membered heteroaromatic ring, substituted or unsubstituted C₃-C₈ carbocycle (including saturated or partially unsaturated situation), substituted or unsubstituted 3 to 8 membered heterocycle (including saturated or partially unsaturated situation); or two adjacent R₁, R₂, or R₃ located at the same ring carbon atom together with the attached ring form saturated or partially unsaturated spiro ring structure, or heterospiro ring structure;
wherein, the ring skeleton of each heterocycle mentioned above may contain 1-3 heteroatoms selected from boron, oxygen, sulfur, phosphorus, and nitrogen; specifically, when the atom is boron, sulfur, phosphorus, or nitrogen, the ring skeleton atom can be oxidized, such as S(O) or S(O)₂.
unless otherwise specified, in the above formulas, the “substituted” refers to the hydrogen atoms on the corresponding group is substituted by one or more substituents selected from the group consisting of: deuterium, tritium, halogen, hydroxy, carboxy, thiol, benzyl, C₁-C₁₂ alkoxycarbonyl, C₁-C₆ aldehyde group, amino, C₁-C₆ amide, nitro, cyano, unsubstituted or halogenated C₁-C₆ alkyl, unsubstituted or halogenated C₃-C₈ cycloalkyl, C₂-C₁₀ alkenyl, C₁-C₆ alkoxy, C₁-C₆ alkyl-amino, C₆-C₁₀ aryl, 5- or 6-membered heteroaryl, 5- or 6-membered non-aromatic heterocyclyl, —O—(C₆-C₁₀ aryl), —O-(5- or 6-membered heteroaryl), C₁-C₁₂ alkylamino carbonyl, unsubstituted or halogenated C₂-C₁₀ acyl, sulfonyl (—SO₂—OH), phosphoryl (—PO₃—OH), unsubstituted or halogenated C₁-C₄ alkyl-S(O)₂— and unsubstituted or halogenated C₁-C₄ alkyl-SO—;
wherein, each chiral atom in the molecule can be in R configuration, S configuration, or a combination thereof.
   Pharmaceutical Composition and Mode of Administration Due to the excellent methyltransferase inhibitory activity of the compound of the present invention, the compound of the invention and various crystal forms, pharmaceutically acceptable inorganic or organic salts, hydrates or solvates thereof, and pharmaceutical composition containing the compound of the present invention as main active ingredients can be used in the treatment, prevention and alleviation of the related diseases induced by the abnormal expression or activity of RC2 complexes, monomers, or combinations thereof.

The pharmaceutical composition of the present invention comprises a safe and effective amount of the compound of the present invention, or a pharmaceutically acceptable salt thereof and pharmaceutically acceptable excipients or carriers. Wherein “safe and effective amount” refers to the amount of compound which is sufficient to significantly improve the condition, and not to generate severe side effects. Generally, the pharmaceutical composition contains 1-2000 mg compound of the invention per dose, preferably, 5-200 mg compound of the invention per dose. Preferably, the “one dose” is one capsule or one pill.

“Pharmaceutically acceptable carrier” means one or more compatible solid or liquid fillers, or gelatinous materials which are suitable for human use and should be of sufficient purity and sufficiently low toxicity. “Compatible” herein refers to the ability of each component of a composition can be mixed with the compound of the present invention and can be mixed with each other without appreciably reducing the efficacy of the compound. Examples of pharmaceutically acceptable carrier include cellulose and derivatives thereof (such as sodium carboxymethylcellulose, sodium ethylcellulose, cellulose acetate, etc.), gelatin, talc, solid lubricant (such as stearic acid, magnesium stearate), calcium sulfate, vegetable oil (such as soybean oil, sesame oil, peanut oil, olive oil, etc.), polyol (such as propylene glycol, glycerol, mannitol, sorbitol, etc.), emulsifier (such as Tween®), wetting agent (such as lauryl sodium sulfate), colorant, flavoring, stabilizer, antioxidant, preservative, pyrogen-free water, etc.

There is no special limitation of administration mode for the compound or pharmaceutical compositions of the present invention, and the representative administration mode includes (but is not limited to): oral, intratumoral, rectal, parenteral (intravenous, intramuscular or subcutaneous), and topical administration.

The solid dosage forms used for oral administration include capsules, tablets, pills, powders, and granules. In these solid dosage forms, the active compounds are mixed with at least one conventional inert excipient (or carrier), such as sodium citrate or dicalcium phosphate, or mixed with any of the following components: (a) fillers or compatibilizer, for example, starch, lactose, sucrose, glucose, mannitol and silicic acid; (b) binders, for example, hydroxymethyl cellulose, alginates, gelatin, polyvinylpyrrolidone, sucrose and arabic gum; (c) humectant, such as, glycerol; (d) disintegrating agents such as agar, calcium carbonate, potato starch or tapioca starch, alginic acid, certain composite silicates, and sodium carbonate; (e) dissolution-retarding agents, such as paraffin; (f) absorption accelerators, for example, quaternary ammonium compounds; (g) wetting agents, such as cetyl alcohol and glyceryl monostearate; (h) adsorbents, for example, kaolin; and (i) lubricants such as talc, stearin calcium, magnesium stearate, solid polyethylene glycol, sodium lauryl sulfate, or the mixtures thereof. In capsules, tablets, and pills, the dosage form may also include buffers.

Solid dosage forms such as tablets, sugar pills, capsules, pills, and granules can be prepared using coating and shell materials, such as casings and other materials well-known in the art. They can contain opacifiers, and the release of active compounds or compounds in the composition can be delayed in a certain part of the digestive tract. Examples of embedding components that may be employed are polymeric substances and waxes. If necessary, the active compound may also be formed into a microcapsule with one or more of the above excipients.

Liquid dosage forms for oral administration include pharmaceutically acceptable lotion, solutions, suspensions, syrups or tinctures. In addition to the active compounds, the liquid dosage forms may contain any conventional inert diluents known in the art such as water or other solvents, solubilizers and emulsifiers, for example, ethanol, isopropanol, ethyl carbonate, ethyl acetate, propylene glycol, 1,3-butanediol, dimethyl carboxamide, as well as oil, in particular, cottonseed oil, peanut oil, corn germ oil, olive oil, castor oil and sesame oil, or the combination thereof.

In addition to these inert diluents, the composition can also include additives such as wetting agents, emulsifiers and suspensions, sweeteners, flavoring agents, and spices.

In addition to active compounds, suspensions can include suspending agents such as ethoxylated isooctadecanol, polyoxyethylene sorbitol and dehydrated sorbitol esters, microcrystalline cellulose, methanol aluminum and agar, or mixtures of these substances.

Compositions for parenteral injection may include physiologically acceptable sterile aqueous or anhydrous solutions, dispersion liquid, suspensions or lotions, and sterile powders for re dissolution into sterile injectable solutions or dispersions. Suitable aqueous and non-aqueous carriers, diluents, solvents or excipients include water, ethanol, polyols, and their suitable mixtures.

The dosage forms of the compounds of the present invention used for local administration include ointments, powders, patches, sprays, and inhalants. The active ingredients are mixed under sterile conditions with physiologically acceptable carriers and any preservatives, buffers, and propellants if necessary.

The compounds of the present invention may be administered alone or in combination with other pharmaceutically acceptable compound. In some preferred embodiments, the compounds of the present invention can form PROTAC with other small molecule compounds, or jointly form ADC with other large molecule compounds such as monoclonal antibodies for application.

When the pharmaceutical compositions are used, a safe and effective amount of compound of the present invention is applied to a mammal (such as human) in need of, wherein the dose of administration is a pharmaceutically effective dose. For a person weighed 60 kg, the daily dose is usually 1-2000 mg, preferably 5-500 mg. Of course, the particular dose should also depend on various factors, such as the route of administration, patient healthy status, which are well within the skills of an experienced physician.

The present invention was further described hereafter in combination with specific examples. It should be understood that these examples are only used to illustrate the and not to limit the scope of the invention. The experimental methods without specific conditions in the following examples generally follow the conventional conditions or the conditions suggested by the manufacturer. Unless otherwise stated, percentages and parts are calculated by weight.

### Example 1: Synthesis of Compound 2-[2-[[4-[(8-chloro-1,7-naphthyridin-2-yl)amino]-1-bicyclo[2.2.2]octyl]methylamino]py rimidin-5-yl]-N-(oxetan-3-yl)acetamide (PA046)

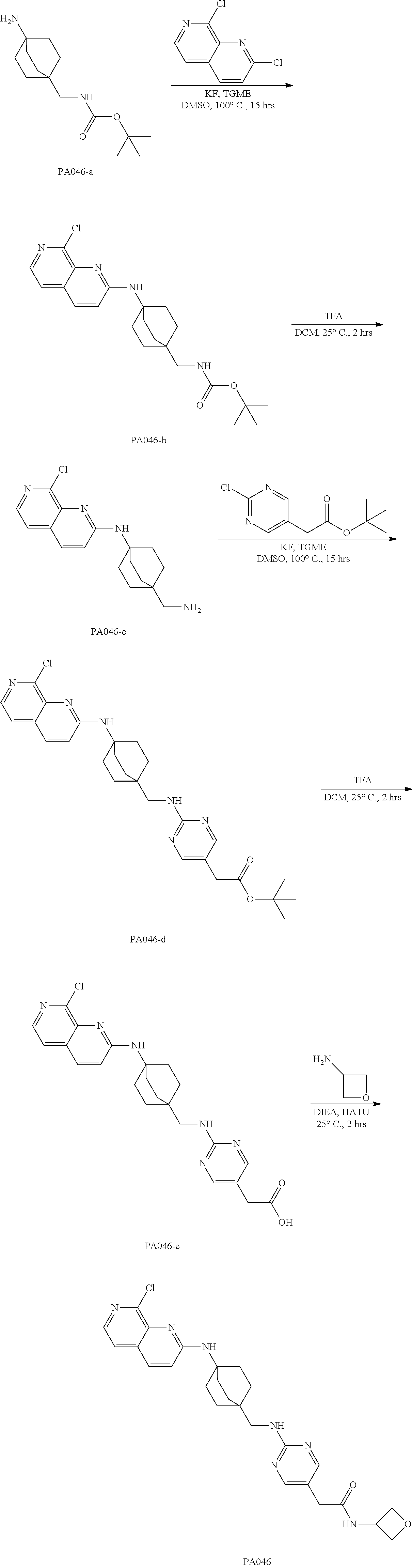

Step 1: To a solution of tert butyl ((4-aminobicyclo [2.2.2]oct-1-yl)carbamate (PA046-a) (100 mg, 0.393 mmol) and 2-chloro-8-methoxy-1,7-naphthyridine (78 mg, 0.393 mmol) in 2 mL of dimethyl sulfoxide was added potassium fluoride (114 mg, 1.97 mmol, 46.05 μL) and 2-[2-(2-methoxyethoxy)ethoxy]ethanol(65 mg, 0.393 mmol). The reaction solution was stirred at 100° C. for 15 hours. LCMS detection showed that the reaction was completed. The reaction solution was dissolved with 10 mL of water and extracted twice with 10 mL of ethyl acetate. The organic phases were combined, dried over magnesium sulfate, filtered, concentrated under reduced pressure, and purified by silica gel column chromatography to obtain tert butyl ((4-((8-chloro-1,7-naphthyridin-2-yl) amino) bicyclo[2.2.2]oct-1-yl)methyl)carbamate (PA046-b) as a yellow solid (150 mg, 91.6% yield). The obtained product was used in the next reaction. LCMS: m/z=417.2 (M+H)+; Step 2: To a solution of tert butyl ((4-((8-chloro-1,7-naphthyridin-2-yl) amino) bicyclo[2.2.2]oct-1-yl)methyl)carbamate(PA046-b) (150 mg, 0.360 mmol) in 2 mL of dichloromethane was added trifluoroacetic acid (41 mg, 0.360 mmol, 26.6 μL). The reaction solution was stirred at 25° C. for 2 hours. LCMS detection showed that the reaction was completed. The reaction solution was concentrated and dried under reduced pressure, then added with 10 mL of 1M hydrochloric acid and extracted twice with 10 mL of ethyl acetate. The inorganic phase was adjusted to pH 8-9 with sodium hydroxide, and then extracted twice with 10 mL of ethyl acetate. The organic phases were combined, dried over magnesium sulfate, filtered, and concentrated under reduced pressure to obtain a crude product of N-[4-(aminomethyl)-1-bicyclo[2.2.2]octyl]-8-chloro-1,7-naphthyridin-2-amino (PA046-c) (110 mg, 96.4% yield) as a white solid, which was directly used in the next reaction without further purification. LCMS: m/z=317.1 (M+H)+;

Step 3: To a solution of N-[4-(aminomethyl)-1-bicyclo[2.2.2]octyl]-8-chloro-1,7-naphthyridin-2-amino (PA046-c) (110 mg, 0.347 mmol) and tert-Butyl 2-(2-chloropyrimidin-5-yl)acetate (95 mg, 0.417 mmol) in dimethyl sulfoxide was added potassium fluoride (101 mg, 1.74 mmol) and 2-[2-(2-methoxyethoxy)ethoxy]ethanol (57 mg, 0.347 mmol). The reaction solution was stirred at 100° C. for 15 hours. LCMS detection showed that the reaction was completed. The reaction solution was dissolved with 10 mL of water and extracted twice with 10 mL of ethyl acetate. The organic phases were combined, dried over magnesium sulfate, filtered, concentrated under reduced pressure and spun-dried, and purified by silica gel column chromatography to obtain a yellow viscous liquid tert butyl 2-[2-[[4-[(8-chloro-1,7-naphthyridin-2-yl)amino]-1-bicyclo[2.2.2]octyl]methylamino]pyrimidin-5-yl]acetate (PA046-d) (140 mg, 79.2% yield). LCMS: m/z=509.3 (M+H)+;

Step 4: To a solution of tert butyl 2-[2-[[4-[(8-chloro-1,7-naphthyridin-2-yl)amino]-1-bicyclo[2.2.2]octyl]methylamino]pyrimidin-5-yl]acetate (PA046-d) (140 mg, 0.275 mmol) in 2 mL of dichloromethane was added trifluoroacetic acid (1.54 g, 13.5 mmol, 1 mL). The reaction was stirred at 25° C. for 2 hours. LCMS detection indicated the generation of target products. After the reaction solution was concentrated and dried under reduced pressure, then added with 10 mL of 1M hydrochloric acid and extracted twice with 10 mL of ethyl acetate. The inorganic phase was adjusted to pH 8 with sodium carbonate, and then extracted twice with 10 mL of ethyl acetate. The organic phases were combined, dried over magnesium sulfate, filtered, and concentrated under reduced pressure to obtain a crude product of 2-[2-[[4-[(8-chloro-1,7-naphthyridin-2-yl) amino]-1-bicyclo [2.2.2]octyl]methylamino]pyrimidin-5-yl]acetic acid (PA046-e) (90 mg, 72.2% yield) as a yellow solid, which was used directly in the next reaction without further purification. LCMS: m/z=453.3 (M+H)+;

Step 5: To a solution of 2-[2-[[4-[(8-chloro-1,7-naphthyridin-2-yl) amino]-1-bicyclo [2.2.2]octyl]methylamino]pyrimidin-5-yl]acetic acid (PA046-e) (90 mg, 0.199 mmol) and oxetan-3-amine (22 mg, 0.298 mmol) in 2 mL of N, N-dimethylformamide was added N, N-diisopropylethylamine (128.41 mg, 993.52 μmol, 173.05 μL, 5 equiv.) dropwise, and finally HATU (151.11 mg, 397.41 μmol, 2 equiv.) was added. The reaction solution was stirred at 25° C. for 2 hours. LCMS detection showed that the reaction was completed. The reaction solution was concentrated and dried under reduced pressure, and then separated by high-performance liquid chromatography column (column: Boston Prime C18 150 mm*30 mm*5 μm; Mobile phase: [water (ammonia solution v/v)−ACN]; B %: 26%-56%, 9 min) to obtain 2-[2-[[4-[(8-chloro-1,7-naphthyridin-2-yl)amino]-1-bicyclo[2.2.2]octyl]methylamino]pyrim idin-5-yl]-N-(oxetan-3-yl)acetamide (PA046) (40 mg, 39.13% yield) as a white solid. LCMS: m/z=508.2 (M+H)+; ¹H NMR (400 MHz, DMSO-d6) δ (ppm) 8.78 (br d, 1H), 8.11 (s, 2H), 7.96 (d, 1H), 7.87 (d, 1H), 7.53 (d, 1H), 7.15 (s, 1H), 7.02 (d, 1H), 6.92 (br t, 1H), 4.73-4.83 (m, 1H), 4.62-4.73 (m, 2H), 4.42 (t, 2H), 3.22-3.27 (m, 2H), 3.13 (br d, 2H), 2.01-2.18 (m, 6H), 1.46-1.65 (m, 6H)

### Example 2: Synthesis of Racemic Compound 2-[2-[[4-[(8-methoxy-1,7-naphthyridin-2-yl)amino]phenyl]methylamino]pyrimidin-5-yl]-N-(oxetan-3-yl) propanamide (PA041), and its Enantiomers PA042 and PA043

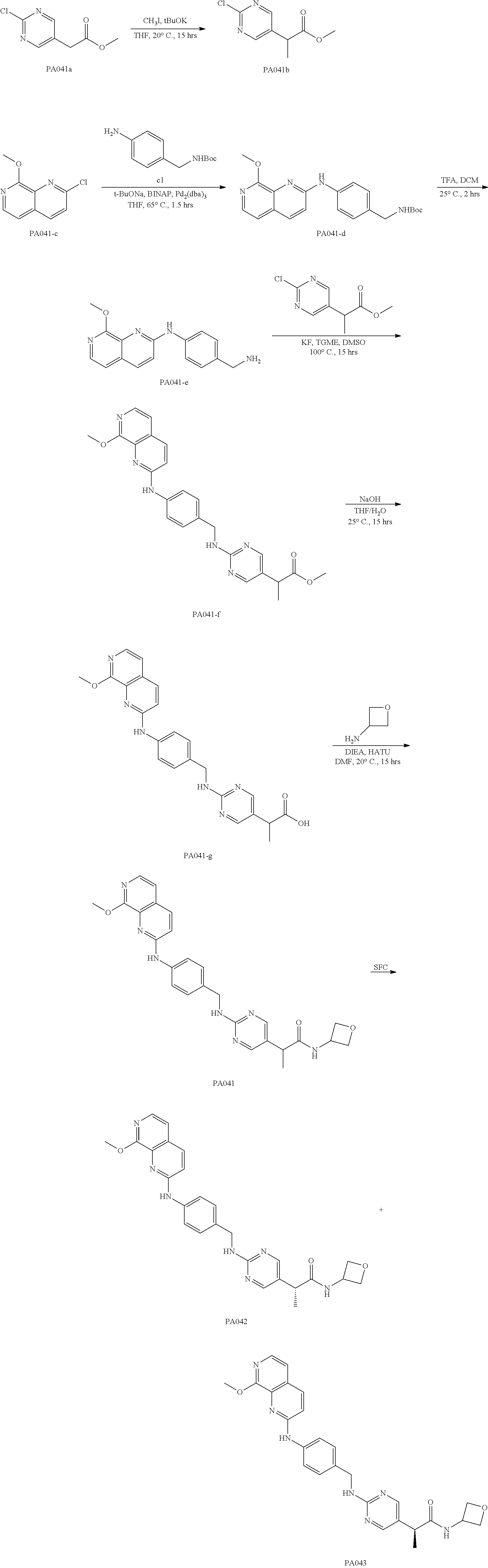

Step 1: To a solution of methyl 2-(2-chloropyrimidin-5-yl)acetate (PA041-a) (170 mg, 0.911 mmol) in anhydrous tetrahydrofuran (5 mL) was added potassium tert butoxide (102 mg, 0.911 mmol). The reaction solution was reacted at 20° C. for 30 minutes, and then iodomethane (129 mg, 0.911 mmol, 57 L) was added under ice water bath. The reaction solution continued stirring under ice water bath for 1 hour. LCMS detection indicated the generation of target product. The reaction solution was added with saturated ammonium chloride solution (30 mL), and extracted with ethyl acetate (30 mL*2). The combined organic phases were dried over anhydrous magnesium sulfate, filtered, and the filtrate was concentrated to obtain a crude product, which was purified by silica gel column chromatography to obtain a colorless viscous crude product of methyl 2-(2-chloropyrimidin-5-yl)propionate (PA041-b) (110 mg, 60.1% purity). LCMS: m/z=200.9 (M+H)+;
Step 2: To a solution of 2-chloro-8-methoxy-1,7-naphthyridine(PA041-c) (200 mg, 1.03 mmol) and tert butyl N-[(4-aminophenyl)methyl]carbamate (c1) (456 mg, 2.06 mmol) in anhydrous tetrahydrofuran (1 mL) was added sodium tert butoxide (98.7 mg, 1.03 mmol), BINAP (191 mg, 0.308 mmol) and Pd₂(dba)₃ (94.1 mg, 0.103 mmol). The reaction solution was heated to 65° C. for 1.5 hours. LCMS showed that the reaction was completed. The reaction solution was added with water (10 mL) and extracted with ethyl acetate (10 mL*2). The combined organic phases were dried over anhydrous magnesium sulfate, filtered, and the filtrate was concentrated to obtain a crude product. The crude product was purified by silica gel column chromatography to obtain a crude product of tert butyl N-[[4-[(8-methoxy-1,7-naphthyridin-2-yl)amino]phenyl]methyl]carbamate (PA041-d) (620 mg, 47.0% purity) as a red solid, which was used directly in the next reaction without purification. LCMS: m/z=381.2 (M+H)+;
Step 3: To a solution of tert butyl N-[[4-[(8-methoxy-1,7-naphthyridin-2-yl)amino]phenyl]methyl]carbamate (PA041-d) (620 mg, 0.815 mmol, 50% purity) in anhydrous dichloromethane (10 mL) was added trifluoroacetic acid (4.62 g, 40.52 mmol, 3 mL). The reaction solution was reacted at 25° C. for 2 hours, and LCMS showed that the reaction was completed. The reaction solution was concentrated under reduced pressure, and then added with hydrochloric acid (10 mL, 1M) and extracted with ethyl acetate (10 mL*2). The aqueous phase was adjusted to pH 8-9 with sodium hydroxide, and extracted with ethyl acetate (10 mL*2). The organic phases were combined and dried over anhydrous magnesium sulfate, filtered, and the filtrate was concentrated to obtain a crude product of N-[4-(aminomethyl)phenyl]-8-methoxy-1,7-naphthyridin-2-amino (PA041-e) (230 mg, 97.29% yield) as a yellow solid, which was used directly in the next reaction without further purification. LCMS: m/z=281.1 (M+H)+;
Step 4: N-[4-(aminomethyl)phenyl]-8-methoxy-1,7-naphthyridin-2-amino (PA041-e) (153 mg, 0.548 mmol), methyl 2-(2-chloropyrimidin-5-yl)propionate (b) (110 mg, 0.548 mmol), potassium fluoride (159 mg, 2.74 mmol, 64 μL), and 2-[2-(2-methoxyethoxy)ethoxy]ethanol (90.0 mg, 0.548 mmol) were added to DMSO (5 mL), and the reaction solution was reacted at 100° C. for 5 hours. LCMS showed that the reaction was completed. The reaction solution was added with water (10 mL), and extracted with ethyl acetate (10 mL*2). The organic phases were combined, dried over anhydrous magnesium sulfate, filtered, and the filtrate was concentrated to obtain a crude product. The crude product was purified by silica gel column chromatography to obtain the product methyl 2-[2-[[4-[(8-methoxy-1,7-naphthyridin-2-yl)amino]phenyl]methylamino]pyrimidin-5-yl]propionate (PA041-f) (230 mg, 82.88% yield, 87.82% purity) as a yellow solid. LCMS: m/z=445.2 (M+H)+;
Step 5: To a solution of methyl 2-[2-[[4-[(8-methoxy-1,7-naphthyridin-2-yl)amino]phenyl]methylamino]pyrimidin-5-yl]propionate (PA041-f) (110 mg, 0.247 mmol) in a mixture of tetrahydrofuran (1 mL) and water (1 mL) was added sodium hydroxide (19.8 mg, 0.495 mmol) under stirring. The reaction solution was allowed to react at 25° C. for 15 hours, and LCMS showed that the reaction was completed. The reaction solution was concentrated under reduced pressure, and then added to dichloromethane (5 mL). The pH was adjusted to 4 with trifluoroacetic acid, and the mixture was concentrated under reduced pressure to obtain a crude product 2-[2-[[4-[(8-methoxy-1,7-naphthyridin-2-yl) amino]phenyl]methylamino]pyrimidin-5-yl]propionic acid (PA041-g) (100 mg, 93.87% yield) as a yellow solid, which was used directly in the next step without further purification. LCMS: m/z=431.2 (M+H)+;
Step 6: To a solution of 2-[2-[[4-[(8-methoxy-1,7-naphthyridin-2-yl) amino]phenyl]methylamino]pyrimidin-5-yl]propionic acid (PA041-g) (100 mg, 0.232 mmol) and oxetan-3-amine (16.9 mg, 0.232 mmol) in DMF (2 mL) was added DIEA (90.0 mg, 0.697 mmol, 121 μL) and HATU (132 mg, 0.348 mmol), and the reaction solution was reacted at 25° C. for 2 hours. LCMS showed that the reaction was completed. The reaction solution was concentrated under reduced pressure, and the residue was purified by reverse phase high performance liquid chromatography (chromatography column: Boston Prime C18 150 mm*30 mm*5 μm; Mobile phase: [water (ammonia v/v)−acetonitrile]; Gradient: 21%-51%, 9 minutes) to obtain racemic compound 2-[2-[[4-[(8-methoxy-1,7-naphthyridin-2-yl)amino]phenyl]methylamino]pyrimidin-5-yl]-N-(oxetan-3-yl) propanamide (PA041) (47 mg) as a yellow solid. LCMS: m/z=486.2 (M+H)+; ¹H NMR (400 MHz, DMSO-d6) δ (ppm) 9.58 (s, 1H), 8.75 (d, 1H), 8.18-8.22 (m, 2H), 8.02 (d, 1H), 7.94 (d, 2H), 7.84 (d, 1H), 7.61 (t, 1H), 7.22-7.29 (m, 4H), 4.64-4.78 (m, 3H), 4.45 (d, 2H), 4.38-4.43 (m, 1H), 4.34 (t, 1H), 4.03 (s, 3H), 3.39-3.46 (m, 1H), 1.30 (d, 3H).
SFC separation was used to obtain two optical enantiomers compounds (chromatographic column: DAICEL CHIRALCEL OD-H (250 mm*30 mm, 5 μm); Mobile phase: [0.1% ammonia ethanol]; B %: 45%-45%, min) (PA042) (18 mg, 15.42% yield) and (PA043) (15 mg, 13.11% yield) as a yellow solid;

(PA042) LCMS: m/z=486.2 (M+H)*; ¹H NMR (400 MHz, DMSO-d6) δ (ppm) 9.57 (s, 1H), 8.73 (br d, J=6.13 Hz, 1H), 8.20 (s, 2H), 8.02 (d, J=8.88 Hz, 1H), 7.93 (br d, J=8.50 Hz, 2H), 7.80-7.89 (m, 1H), 7.58 (br t, J=6.25 Hz, 1H), 7.16-7.35 (m, 4H), 4.55-4.80 (m, 3H), 4.45 (br d, J=6.25 Hz, 2H), 4.39-4.42 (m, 1H), 4.34 (br t, J=5.94 Hz, 1H), 4.03 (s, 3H), 3.44-3.47 (m, 1H), 1.30 (br d, J=7.13 Hz, 3H).

(PA043) LCMS: m/z=486.2 (M+H)+; ¹H NMR (400 MHz, DMSO-d6) δ (ppm) 9.57 (s, 1H), 8.73 (br d, 1H), 8.20 (s, 2H), 8.02 (d, 1H), 7.93 (br d, 2H), 7.84 (d, 1H), 7.58 (br t, 1H), 7.16-7.34 (m, 4H), 4.55-4.80 (m, 3H), 4.45 (br d, 2H), 4.37-4.43 (m, 1H), 4.30-4.37 (m, 1H), 4.03 (s, 3H), 3.46 (br s, 1H), 1.30 (br d, 3H).

### Example 3: Synthesis of Racemic Compound 2-(2-((1-(4-((8-methoxy-1,7-naphthyridin-2-yl) amino) phenyl) ethyl) amino) pyrimidin-5-yl) acetic acid N-(oxetan-3-yl) acetamide (PA039) and its Enantiomers PA061 and PA062

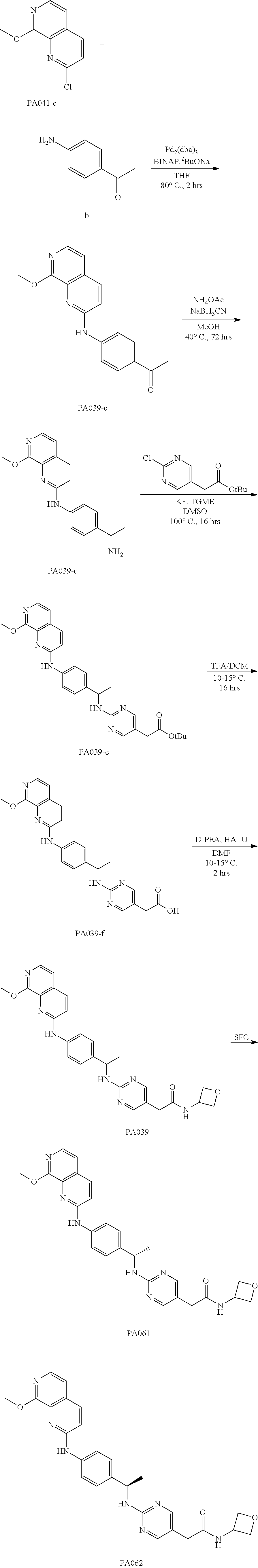

Step 1: 1-(4-aminophenyl)ethane 1-(4-aminophenyl) ethanone (b) (180 mg, 1.33 mmol), 2-chloro-8-methoxy-1,7-naphthyridine (PA041-c) (250 mg, 1.28 mmol), sodium tert butoxide (140 mg, 1.46 mmol) and anhydrous tetrahydrofuran (10 mL) were added to a reaction flask, and the reaction flask was degassed to remove oxygen under reduced pressure and replaced several times with nitrogen, and then BINAP (160 mg, 257 μmol) and Pd₂(dba)₃ (100 mg, 109 μmol) were added to the reaction solution. The reaction was carried out at 80° C. for 2 hours. LCMS detection indicated the generation of target products. The reaction was cooled to room temperature, added with saturated saline solution (10 mL), and extracted twice with dichloromethane (15 mL). The combined organic phase was dried with anhydrous sodium sulfate, and filtered; the filtrate was concentrated under reduced pressure to obtain a crude product. The crude product was purified by silica gel column chromatography to obtain the compound 1-(4-((8-methoxy-1,7-naphthyridin-2-yl)amino)phenyl)ethanone (PA039-c) (392 mg, 94.6% yield) as an orange solid. LCMS: m/z=294.0 [M+1]+; ¹H NMR (400 MHz, DMSO-d6) δ (ppm) 10.08 (s, 1H), 8.19 (d, 2H), 8.13 (d, 1H), 7.97 (d, J=8.63 Hz, 2H), 7.91 (d, 1H), 7.33 (d, 1H), 7.29 (d, 1H), 4.08 (s, 3H), 2.54 (s, 3H)
Step 2: To a solution of 1-(4-((8-methoxy-1,7-naphthyridin-2-yl)amino)phenyl)ethanone (PA039-c) (360 mg, 1.23 mmol) and ammonium acetate (1.89 g, 24.47 mmol) in methanol (30 mL) was added sodium cyanoborohydride(308 mg, 4.90 mmol). The reaction solution was reacted at 40° C. for 16 hours. LCMS showed the generation of the target product and the remaining raw materials. 300 mg sodium cyanoborohydride was added and the reaction solution was reacted at 40° C. for 48 hours. LCMS showed that the reaction was complete. The reaction solution was concentrated under reduced pressure, and 50 mL of saturated saline solution was added. The mixture was extracted with methanol/dichloromethane (1:10, 75 mL), and the aqueous phase was extracted twice with dichloromethane (50 mL). The combined organic phases were dried over anhydrous magnesium sulfate, and filtered, and the filtrate was concentrated to obtain a crude product of N-(4-(1-aminoethyl) phenyl)-8-methoxy-1,7-naphthyridin-2-amino (PA039-d) (372 mg) as a yellow solid, which was directly used in the next reaction without further purification. LCMS m/z=295.1 [M+1]+;
Step 3: To a solution of N-(4-(1-aminoethyl) phenyl)-8-methoxy-1,7-naphthyridin-2-amino (PA039-d) (150 mg, 0.51 mmol) and tert-Butyl 2-(2-chloropyrimidin-5-yl)acetate (d1) (115 mg, 0.505 mmol) in DMSO (4 mL) was added 2-[2-(2-methoxyethoxy)ethoxy]ethanol (TGME) (92 mg, 0.561 mmol) and potassium fluoride (148 mg, 2.55 mmol), and the reaction solution was reacted at 100° C. for 16 hours. LCMS showed that the reaction was completed. The reaction solution was cooled to room temperature, diluted with water (25 mL), and filtered, and the filter cake was washed with water (20 mL). The filter cake was collected and lyophilized to obtain a crude product of tert butyl 2-(2-((1-(4-((8-methoxy-1,7-naphthyridin-2-yl) amino) phenyl) ethyl) amino) pyrimidin-5-yl) acetate (PA039-e) (183 mg) as a brown solid. LCMS m/z=487.2[M+1]+;
Step 4: A mixture of tert butyl 2-(2-((1-(4-((8-methoxy-1,7-naphthyridin-2-yl) amino) phenyl) ethyl) amino) pyrimidin-5-yl) acetate (PA039-e) (40 mg, 0.082 mmol) and trifluoroacetic acid/dichloromethane (3 mL) [trifluoroacetic acid/dichloromethane=1:5] was reacted at 10-15° C. for 16 hours. LCMS detection indicated the generation of target products, the reaction solution was concentrated under reduced pressure to obtain a crude product, then slurried with methyl tert butyl ether (10 mL), and diluted with petroleum ether (10 mL), and the solution was allowed to stand still and remove the solution. The residue was dried under reduced pressure to obtain a crude product of 2-(2-((1-(4-((8-methoxy-1,7-naphthyridin-2-yl)amino)phenyl)ethyl) amino)pyrimidin-5-yl)acetic acid (PA039-f) (82 mg, trifluoroacetate) as a brown slurry-like substance, which was used directly for the next reaction without further purification. LCMS m/z=431.1 [M+1]+;
Step 5: To a solution of 2-(2-((1-(4-((8-methoxy-1,7-naphthyridin-2-yl)amino)phenyl)ethyl) amino)pyrimidin-5-yl)acetic acid (PA039-f) (82 mg, 0.075 mmol) in DMF (1 mL) was added oxetan-3-amine (10 mg, 0.137 mmol), DIPEA (50 mg, 0.387 mmol) and HATU (45 mg, 0.118 mmol). The reaction solution was reacted at 10-15° C. for 2 hours. LCMS detection indicated the generation of target products. The reaction solution was concentrated under reduced pressure to obtain a crude product, which was then purified by reverse phase high performance liquid chromatography (chromatographic column: YMC Triart preparative C18 150*40 mm*7 μm; Mobile phase: [Water (ammonia water+ammonium bicarbonate)-acetonitrile]; Gradient: 7%-47%, 9 minutes), and lyophilized to obtain the product 2-(2-((1-(4-((8-methoxy-1,7-naphthyridin-2-yl)amino)phenyl)ethyl)pyrimidin-5-yl)acetic acid N-(oxetan-3-yl)acetamide (PA039) (12 mg, 0.024 mmol, 31.8% yield) as an off-white solid. LCMS m/z=486.2 [M+1]+; ¹H NMR (400 MHz, DMSO-d6) δ (ppm) 9.56 (s, 1H), 8.78 (d, 1H), 8.11 (s, 2H), 8.01 (d, 1H), 7.91 (d, 2H), 7.83 (d, 1H), 7.51 (d, 1H), 7.33 (d, 2H), 7.18-7.25 (m, 2H), 5.00-5.09 (m, 1H), 4.70-4.80 (m, 0.01H), 4.64-4.69 (m, 2H), 4.39 (t, 2H), 4.03 (s, 3H), 3.22 (s, 2H), 1.43 (d, 3H)
Step 6: 70 mg of 2-(2-((1-(4-((8-methoxy-1,7-naphthyridin-2-yl)amino)phenyl)ethyl)pyrimidin-5-yl)acetic acid N-(oxetan-3-yl)acetamide (PA039) was subjected to chiral separation (chromatographic column: DAICEL CHIRALCEL OD-H (250 mm*30 mm, 5 μm); Mobile phase: [0.1% ammonia ethanol]; B %: 45%-45%, min) to obtain two optical enantiomers: white solid PA061 (25 mg, 35.7% yield). LCMS m/z=486.1 [M+1]+; Chiral SFC: ee %=98.32%, ¹H NMR (400 MHz, DMSO-d6) δ 9.56 (s, 1H), 8.78 (d, 1H), 8.11 (s, 2H), 8.01 (d, 1H), 7.91 (d, 2H), 7.83 (d, 1H), 7.51 (d, 1H), 7.33 (d, 2H), 7.18-7.25 (m, 2H), 5.00-5.09 (m, 1H), 4.70-4.80 (m, 1H), 4.64-4.69 (m, 2H), 4.39 (t, 2H), 4.03 (s, 3H), 3.22 (s, 2H), 1.43 (d, H); white solid compound PA062 (25 mg, yield 35.7%) LCMS: m/z=486.1 [M+1]+; Chiral SFC ee %=87.62%; ¹H NMR (400 MHz, DMSO-d6) δ 9.56 (s, 1H), 8.78 (d, 1H), 8.11 (s, 2H), 8.01 (d, 1H), 7.91 (d, 2H), 7.83 (d, 1H), 7.51 (d, 1H), 7.33 (d, 2H), 7.18-7.25 (m, 2H), 5.00-5.09 (m, 1H), 4.70-4.80 (m, 1H), 4.64-4.69 (m, 2H), 4.39 (t, 2H), 4.03 (s, 3H), 3.22 (s, 2H), 1.43 (d, 3H)

### Example 4: Synthesis of Compound (S)-3-(2-(1-(4-(8-chloro-7-methylquinolin-2-amino)-3-fluorophenyl)ethylamino) pyrimidin-5-ylmethylamino)thietan-1,1-dione (PA167)

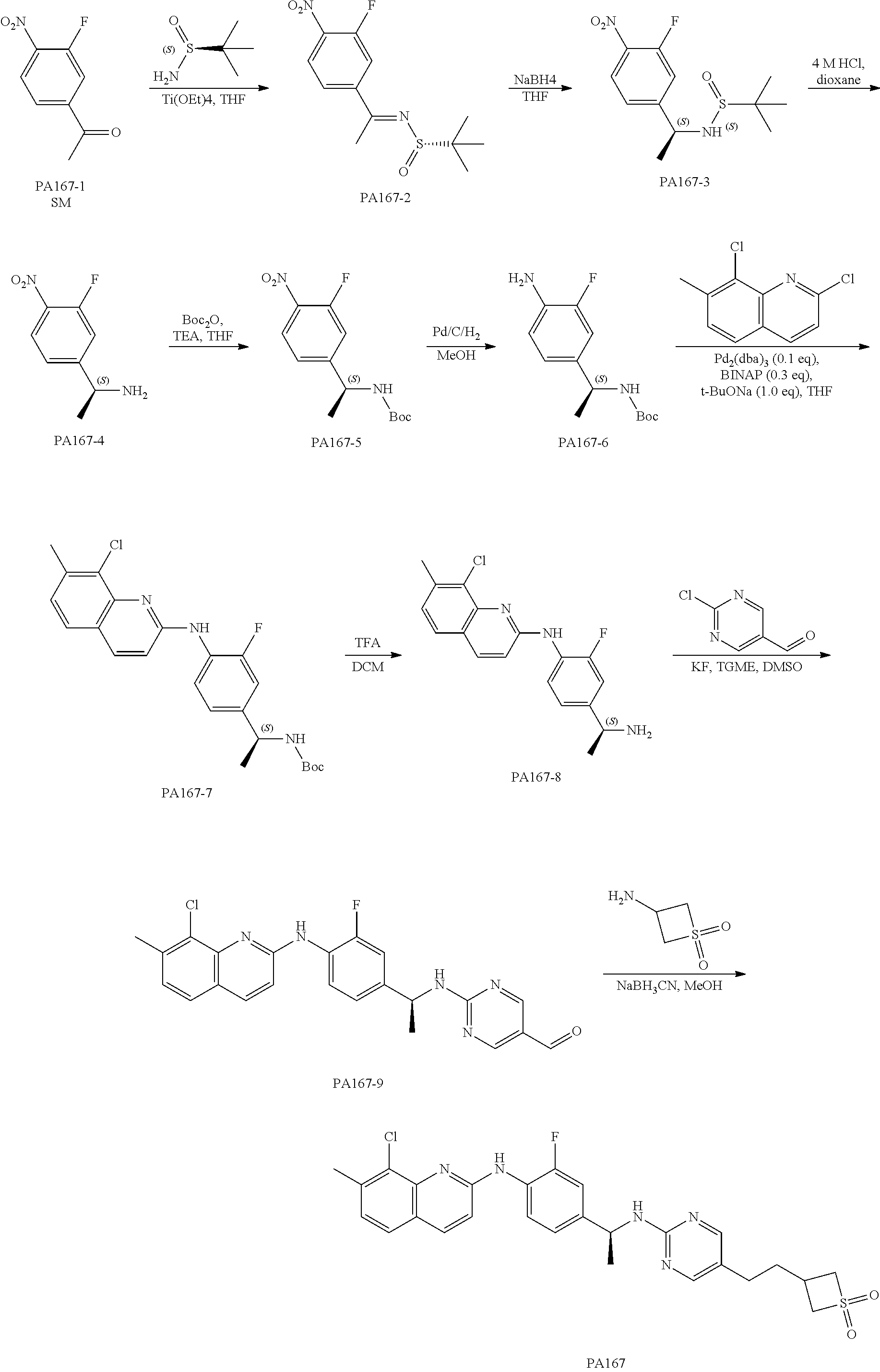

### 1. Preparation of Compound (S, E)-N-(1-(3-fluoro-4-nitrophenylethylene)-2-methylpropan-2-sulfimide (PA167-2)

Compound 1-(3-fluoro-4-nitrophenyl)ethanone(PA167-1) (5.00 g, 27.3 mmol) was dissolved in 50 mL of tetrahydrofuran, and (S)-tert butyl sulfinamide(4.96 g, 41.0 mmol) and Ti(OEt)₄ (12.46 g, 54.6 mmol, 11.4 mL) were added. The reaction solution was stirred at 75° C. for 16 hours, cooled to room temperature, and then quenched with ice-water (30 mL). 100 mL of ethyl acetate was added and the mixture was stirred at room temperature for 15 minutes. After filtration, the filter cake was washed with 3*20 ml of ethyl acetate. The combined organic phases were washed with saturated saline solution, dried over anhydrous sodium sulfate, and concentrated to obtain 7.80 g yellow crude product of (S, E)-N-(1-(3-fluoro-4-nitrophenylethylene)-2-methylpropan-2-sulfimide (PA167-2). (ESI) m/z. [M+H]+ 287.0.

### 2. Preparation of (S)-N-((S)-1-(3-fluoro-4-nitrophenyl)ethyl)-2-methylpropane-2-sulfimide (PA167-3)

The crude product (S, E)-N-(1-(3-fluoro-4-nitrophenylethylene)-2-methylpropan-2-sulfimide (PA167-2) (7.8 g, 27.2 mmol) was dissolved in 100 mL of tetrahydrofuran, and then cooled to −50° C. Sodium borohydride (4.12 g, 109.0 mmol) was added in batches. The reaction mixture was slowly warmed to 0-25° C. and stirred for 3 hours, and 20 mL of saturated ammonium chloride aqueous solution was added carefully to quench the reaction. The mixture was extracted with ethyl acetate. The organic phase was washed with saturated saline solution and dried over anhydrous sodium sulfate, filtered, concentrated, and purified by silica gel column chromatography to obtain (S)-N-((S)-1-(3-fluoro-4-nitrophenyl)ethyl)-2-methylpropane-2-sulfimide (PA167-3) (5.17 g, 17.9 mmol, 65.8% yield) as a yellow oil. (ESI) m/z. [M+H]+ 289.0.

### 3. Synthesis of Compound (S)-1-(3-fluoro-4-nitrophenyl)ethyl-1-amine (PA167-4)

To a solution of compound (S)-N-((S)-1-(3-fluoro-4-nitrophenyl)ethyl)-2-methylpropane-2-sulfimide (PA167-3) (1.0 g, 3.47 mmol) in 8 mL of 1,4-dioxane was added dropwise 8 mL of 4M hydrochloric acid. The reaction solution was stirred at room temperature for 3 hours, and the reaction was complete. After removing the solvent, added with water and extracted with ethyl acetate. The organic phase was washed with saturated aqueous sodium bicarbonate solution, dried over anhydrous sodium sulfate, filtered, and concentrated to obtain 640 mg of crude product of (S)-1-(3-fluoro-4-nitrophenyl)ethyl-1-amine (PA167-4) as a yellow mucus. (ESI) m/z. [M+H]+ 185.0.

### 4. Synthesis of Compound tert butyl (S)-(1-(3-fluoro-4-nitrophenyl)ethyl)carbonate (PA167-5)

To a solution of (S)-1-(3-fluoro-4-nitrophenyl)ethyl-1-amine (PA167-4) (650 mg, 3.5 mmol) in 5 mL of tetrahydrofuran was added Boc₂O (1.54 g, 7.06 mmol) and triethylamine (1.07 g, 10.59 mmol, 3.77 mL). The reaction solution was stirred at room temperature for 2 hours. The reaction solution was concentrated, added with water and extracted with ethyl acetate. The organic phase was washed with saturated saline solution, dried over anhydrous sodium sulfate, filtered, concentrated, and purified by silica gel column chromatography to obtain tert butyl (S)-(1-(3-fluoro-4-nitrophenyl)ethyl)carbonate (PA167-5) (700.0 mg, 2.5 mmol, 69.8% yield) as a yellow solid. (ESI) m/z. [M+H]+ 229.0.

### 5. Synthesis of Compound tert butyl (S)-(1-(4-amino-3-fluoro-phenyl)ethyl) carbonate (PA167-6)

To a solution of tert butyl (S)-(1-(3-fluoro-4-nitrophenyl)ethyl)carbonate (PA167-5)(700.0 mg, 2.5 mmol) in 10 mL of methanol was added 70 mg of Pd/C. The mixture was stirred at room temperature for 2 hours and reacted completely. The reaction mixture was filtered through diatomaceous earth and washed with methanol. The organic phase was concentrated to obtain a crude product of tert butyl (S)-(1-(4-amino-3-fluoro-phenyl)ethyl) carbonate (PA167-6) as a yellow mucus (540.0 mg, 2.1 mmol, 86.2% yield). (ESI) m/z. [M+H]+ 277.

### 6. Synthesis of Compound tert butyl (S)-(1-(4-((8-chloro-7-methylquinolin-2-yl)amino)-3-fluoro-phenyl)ethyl)carbonate (PA167-7)

To a mixture of 2,8-dichloro-7-methylquinoline (366.9 mg, 1.7 mmol), tert butyl (S)-(1-(4-amino-3-fluoro-phenyl)ethyl) carbonate (PA167-6) (PA167-6) (400.0 mg, 1.6 mmol), sodium tert butoxide (151.2 mg, 1.6 mmol), and BINAP (293.8 mg, 471.9 μmol) in 10 mL of dry tetrahydrofuran was added Pd₂(dba)₃ (144.0 mg, 157.3 μmol) after nitrogen displacement. The reaction was stirred at 65° C. for 16 hours under nitrogen protection. The reaction solution was filtered, and the filtrate was added with 100 mL of water and extracted twice with ethyl acetate. The combined organic phases were washed with saturated saline, dried over anhydrous sodium sulfate, filtered, concentrated, and purified by silica gel column chromatography to obtain compound tert butyl (S)-(1-(4-((8-chloro-7-methylquinolin-2-yl)amino)-3-fluoro-phenyl)ethyl)carbonate (PA167-7)(400.0 mg, 651.3 μmol, 41.4% yield) as a brown solid. (ESI) m/z. [M+H]+ 430.2

### 7. Synthesis of Compound (S)-N-(4-(1-aminoethyl)-2-fluorophenyl)-8-chloro-7-methylquinolin-2-amine (PA167-8)

To a solution of tert butyl (S)-(1-(4-((8-chloro-7-methylquinolin-2-yl)amino)-3-fluoro-phenyl)ethyl)carbonate (PA167-7) (400.0 mg, 93.4 μmol) in 3 mL of dichloromethane was added dropwise 5 mL of trifluoroacetic acid at room temperature under stirring and stirred for 2 hours. The reaction mixture was concentrated and diluted with water, and the pH was adjusted to 8-9 using a saturated sodium carbonate aqueous solution. The mixture was then extracted with dichloromethane, and the combined organic phases were dried over anhydrous sodium sulfate, filtered, concentrated, and purified by silica gel column chromatography to obtain compound (S)-N-(4-(1-aminoethyl)-2-fluorophenyl)-8-chloro-7-methylquinolin-2-amine (PA167-8)(190.0 mg, 518.5 μmol, 55.7% yield) as a yellow solid. (ESI) m/z. [M+H]+ 330.0.

### 8. Synthesis of Compound (S)-2-((1-(4-(8-chloro-7-methylquinolin-2-yl)amino)-3-fluorophenyl)ethyl)amino) pyrimidin-5-carbaldehyde (PA167-9)

Under nitrogen protection, to a mixture of (S)-N-(4-(1-aminoethyl)-2-fluorophenyl)-8-chloro-7-methylquinolin-2-amine (PA167-8) (90 mg, 272.9 μmol), 2-chloropyrimidin-5-carbaldehyde (38.9 mg, 272.9 μmol), TGME (44.8 mg, 272.9 μmol) was added potassium fluoride (79.3 mg, 1.4 mmol) and 2 mL of dimethyl sulfoxide. The reaction mixture was heated to 110° C. and stirred for 16 hours. The reaction mixture was added with 10 mL of water and extracted with ethyl acetate. The combined organic phases were washed with saturated saline solution, dried over anhydrous sodium sulfate, filtered, concentrated, and separated by silica gel column chromatography to obtain (S)-2-((1-(4-(8-chloro-7-methylquinolin-2-yl)amino)-3-fluorophenyl)ethyl)amino) pyrimidin-5-carbaldehyde (PA167-9) (55.0 mg, 118.6 μmol, 43.5% yield) as a yellow solid. (ESI) m/z. [M+H]+ 436.2.

### 9. Synthesis of Compound (S)-3-(((2-((1-(4-(8-chloro-7-methylquinolin-2-amino)-3-fluorophenyl)ethyl)amino) pyrimidin-5-yl)methyl)amino)thietan-1,1-dione (PA167)

Compound (S)-2-((1-(4-(8-chloro-7-methylquinolin-2-yl)amino)-3-fluorophenyl) ethyl) amino) pyrimidin-5-carbaldehyde (PA167-9)(55.0 mg, 126.2 μmol), and 1,1-dioxothietan-3-amine (19.9 mg, 164.0 μmol) were dissolved in 3 mL of dry methanol and stirred at 50° C. for 18 hours before cooling to room temperature. The reaction mixture was added with 1 mL of dichloromethane and sodium cyanoborohydride (15.9 mg, 252.4 μmol), and stirred at room temperature for 5 hours. The reaction was quenched by adding 2 drops of water. After post-treatment, the reaction solution was added with methanol and purified by preparative HPLC to obtain compound (S)-3-(((2-((1-(4-(8-chloro-7-methylquinolin-2-amino)-3-fluorophenyl)ethyl)amino) pyrimidin-5-yl)methyl)amino)thietan-1,1-dione (PA167) as a white solid. ¹H NMR (400 MHz, DMSO-d6) δ 9.20 (d, 1H), 9.08 (t, 1H), 8.41 (s, OH), 8.16 (s, 2H), 8.04 (d, 1H), 7.59 (dd, 2H), 7.30 (d, 1H), 7.27-7.17 (m, 3H), 5.05 (p, 1H), 4.28-4.20 (m, 2H), 3.84 (ddd, 2H), 3.46-3.40 (m, 1H), 3.36 (s, 2H), 2.49 (s, 3H), 1.41 (d, 3H); (ESI) m z. [M+H]⁺ 541.2.

### Example 5: Synthesis of Compound 2-(2-(((S)-1-(4-((8-chloro-1,7-naphthyridin-2-yl) amino)bicyclic[2.2.2]octan-1-yl)ethyl)amino)pyrimidin-5-yl) propionic acid (PA169-11) and Diastereomers PA166, PA207

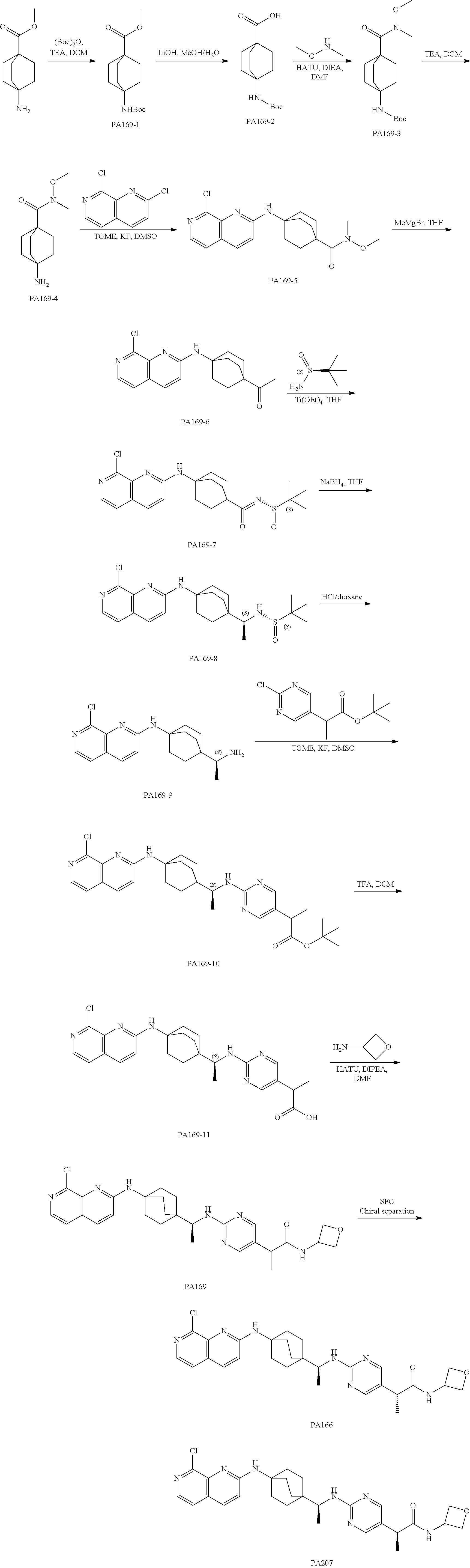

### 1. Synthesis of Compound methyl 4-(tert butoxycarbonylamino)bicyclo[2.2.2]octan-1-carboxylate (PA169-1)

To a solution of methyl 4-aminobicyclo[2.2.2]octan-1-carboxylate (2 g, 10.9 mmol) in 20 mL of dichloromethane was added triethylamine (3.31 g, 32.7 mmol) and di tert butyl carbonate (2.86 g, 13.1 mmol). The reaction mixture was stirred at room temperature for 16 hours. The reaction was added with water and extracted with dichloromethane. The organic phase was washed with saturated saline solution, extracted with dichloromethane, dried over anhydrous sodium sulfate, filtered and concentrated to obtain 2.20 g of white crude product methyl 4-(tert butoxycarbonylamino)bicyclo[2.2.2]octan-1-carboxylate (PA169-1). (ESI) m/z. [M+H]+ 228.0.

### 2. Synthesis of Compound 4-(tert butoxycarbonylamino)bicyclo[2.2.2]octan-1-carboxylic acid (PA169-2)

To a solution of methyl 4-(tert butoxycarbonylamino)bicyclo[2.2.2]octan-1-carboxylate (PA169-1) (2.2 g, 7.76 mmol) in 15 mL of methanol was added 5 mL of water and lithium hydroxide (371.86 mg, 15.53 mmol). The mixture was stirred at room temperature for 4 hours. The reaction solution was adjusted with 1N hydrochloric acid solution to pH 6, extracted with ethyl acetate and washed with saturated saline solution. The organic phase was dried over anhydrous sodium sulfate, filtered, and concentrated to obtain the white crude product 4-(tert butoxycarbonylamino)bicyclo[2.2.2]octan-1-carboxylic acid (PA169-2). (ESI) m/z. [M+H]+ 214.0.

### 3. Synthesis of Compound tert butyl N-[4-[methoxy(methyl)carbamoyl]-1-bicyclo [2.2.2]octyl]carbonate (PA169-3)

To a solution of 4-(tert butoxycarbonylamino)bicyclo[2.2.2]octan-1-carboxylic acid (PA169-2) (1.7 g, 6.31 mmol) and diisopropylethylamine (1.63 g, 12.62 mmol) in 20 mL of DMF was added N,O-dimethylhydroxylamine (424.10 mg, 6.94 mmol) and HATU (2.64 g, 6.94 mmol), and the reaction mixture was stirred at room temperature for 1 hour. The reaction solution was filtered, diluted with water, and extracted with ethyl acetate. The organic phase was washed with saturated saline solution, dried over anhydrous sodium sulfate, filtered, concentrated, and purified by silica gel column chromatography to obtain compound tert butyl N-[4-[methoxy(methyl)carbamoyl]-1-bicyclo[2.2.2]octyl]carbonate (PA169-3) (1.60 g, 4.61 mmol, 73.03% yield, 90% purity) as a white solid. (ESI) m/z. [M+H]+ 313.20.

### 4. Synthesis of Compound 4-amino-N-methoxy-N-methylbicyclo[2.2.2]octyl-1-carboxamide (PA169-4)

To a solution of Compound tert butyl N-[4-[methoxy(methyl)carbamoyl]-1-bicyclo [2.2.2]octyl]carbonate (PA169-3) (1.6 g, 5.12 mmol) in 10 mL of dichloromethane was added 3 mL of trifluoroacetic acid. The reaction solution was stirred at room temperature for 2 hours. The reaction solution was concentrated, and sodium bicarbonate aqueous solution was added to adjust the pH to 10. The mixture was extracted with dichloromethane and washed with saturated saline solution, dried over anhydrous sodium sulfate, filtered and concentrated to obtain 1.00 g of crude product of 4-amino-N-methoxy-N-methylbicyclo[2.2.2]octyl-1-carboxamide (PA169-4) as a white solid. (ESI) m/z. [M+H]+ 213.0.

### 5. Synthesis of Compound 4-[(8-chloro-1,7-naphthyridin-2-yl)amino]-N-methoxy-N-methyl-bicyclo[2.2.2]octyl-1-carboxamide (PA169-5)

To a solution of compound 4-amino-N-methoxy-N-methylbicyclo[2.2.2]octyl-1-carboxamide (PA169-4) (0.95 g, 4.48 mmol) in 15 mL of dimethyl sulfoxide was added 2,8-dichloro-1,7-naphthyridine (1.25 g, 6.27 mmol), potassium fluoride (1.30 g, 22.38 mmol), and TGME (734.80 mg, 4.48 mmol). The reaction solution was heated to 100° C. and stirred for 16 hours. The reaction solution was filtered, added with 30 mL of water and extracted with ethyl acetate. The organic phase was washed with saturated saline solution, dried over anhydrous sodium sulfate, filtered and concentrated. The crude product was purified by silica gel column chromatography to obtain compound 4-[(8-chloro-1,7-naphthyridin-2-yl)amino]-N-methoxy-N-methyl-bicyclo[2.2.2]octyl-1-carboxamide (PA169-5) (1.00 g, 2.40 mmol, 53.6% yield, 90% purity) as a white solid. (ESI) m/z. [M+H]+ 375.0.

### 6. Synthesis of Compound 1-[4-[(8-chloro-1,7-naphthyridin-2-yl)amino]-1-bicyclo [2.2.2]octyl]ethanone (PA169-6)

To a solution of Compound 4-[(8-chloro-1,7-naphthyridin-2-yl)amino]-N-methoxy-N-methyl-bicyclo[2.2.2]octyl-1-carboxamide (PA169-5) (1 g, 2.67 mmol) in 10 mL of dry tetrahydrofuran was added dropwise a solution of methyl magnesium bromide tetrahydrofuran (3M, 4.45 mL) after cooling to 0° C. under nitrogen protection. The reaction solution was slowly warmed to 40° C. and reacted for 16 hours. The reaction solution was added dropwise carefully with 15 mL of saturated ammonium chloride aqueous solution, extracted with ethyl acetate, washed with saturated saline solution, dried over anhydrous sodium sulfate, filtered and concentrated to obtain 800 mg crude product of 1-[4-[(8-chloro-1,7-naphthyridin-2-yl)amino]-1-bicyclo [2.2.2]octyl]ethanone (PA169-6) as a yellow solid. (ESI) m/z. [M+H]+ 330.10.

### 7. Synthesis of Compound (S, E)-N-(1-(4-((8-chloro-1,7-naphthyridin-2-yl) amino)bicyclo[2.2.2]octan-1-yl)ethylene)-2-methylpropan-2-sulfinamide (PA169-7)

To a solution of compound 1-[4-[(8-chloro-1,7-naphthyridin-2-yl)amino]-1-bicyclo [2.2.2]octyl]ethanone (PA169-6) (0.15 g, 454.8 μmol) in 10 mL of dry tetrahydrofuran was added tetraethoxytitanium(207.5 mg, 909.6 μmol), and the reaction mixture was heated to 75° C. and stirred for 16 hours. The reaction solution was added with 20 mL of ethyl acetate and 10 mL of water after cooling. The reaction solution was stirred for another half hour and then extracted with ethyl acetate. The organic phase was washed with saturated saline and dried over anhydrous sodium sulfate, filtered and concentrated to obtain 150 mg crude product of (S, E)-N-(1-(4-((8-chloro-1,7-naphthyridin-2-yl) amino)bicyclo[2.2.2]octan-1-yl)ethylene)-2-methylpropan-2-sulfinamide (PA169-7) as a white solid. (ESI) m/z. [M+H]+ 433.20.

### 8. Synthesis of Compound (S)-N-((S)-1-(4-((8-chloro-1,7-naphthyridin-2-yl)amino) bicyclo[2.2.2]octan-1-yl)ethyl)-2-methylpropan-2-sulfinamide (PA169-8)

Compound (S, E)-N-(1-(4-((8-chloro-1,7-naphthyridin-2-yl)amino)bicycle [2.2.2]octan-1-yl)ethylene)-2-methylpropan-2-sulfinamide (PA169-7) (0.15 g, 346.4 μmol) was dissolved in 5 mL of tetrahydrofuran. The mixture was cooled to −50° C., and added with sodium borohydride (52.4 mg, 1.39 mmol). The reaction solution was slowly warmed to room temperature and stirred for 3 hours. The reaction solution was quenched by adding 10 mL of saturated ammonium chloride aqueous solution, extracted with ethyl acetate, washed with saturated saline solution, dried over anhydrous sodium sulfate, filtered and concentrated to obtain 120 mg crude product of (S)-N-((S)-1-(4-((8-chloro-1,7-naphthyridin-2-yl)amino) bicycle [2.2.2]octan-1-yl)ethyl)-2-methylpropan-2-sulfinamide (PA169-8) as a yellow solid. (ESI) m/z. [M+H]+ 435.1.

### 9. Synthesis of Compound (S)-N-(4-(1-aminoethyl)bicyclic[2.2.2]octan-1-yl)-8-chloro-1,7-naphthyridin-2-amine (PA169-9)

Compound (S)-N-((S)-1-(4-((8-chloro-1,7-naphthyridin-2-yl)amino) bicyclo[2.2.2]octan-1-yl)ethyl)-2-methylpropan-2-sulfinamide (PA169-8) (0.12 g, 275.8 μmol) was dissolved in 5 mL of 1,4-dioxane. The mixture was cooled to 0° C., and then hydrochloric acid solution (1,4-dioxane solution, 2M concentration, 1.1 mL) was added dropwise. The reaction solution was slowly warmed to room temperature and stirred for another 3 hours until the reaction was complete. The reaction solution was concentrated, saturated sodium bicarbonate aqueous solution was added to adjust pH to 8 and the mixture was extracted with ethyl acetate. The organic phase was washed with saturated saline solution, dried over anhydrous sodium sulfate, filtered and concentrated to obtain 75 mg crude product of (S)-N-(4-(1-aminoethyl)bicyclic[2.2.2]octan-1-yl)-8-chloro-1,7-naphthyridin-2-amine (PA169-9) as a yellow solid. (ESI) m/z. [M+H]+ 331.0.

### 10. Synthesis of Compound tert butyl 2-(2-(((S)-1-(4-((8-chloro-1,7-naphthyridin-2-yl)amino) bicyclo[2.2.2]octan-1-yl)ethyl)amino)pyrimidin-5-yl)propionate (PA169-10)

To a mixture of (S)-N-(4-(1-aminoethyl)bicyclic[2.2.2]octan-1-yl)-8-chloro-1,7-naphthyridin-2-amine (PA169-9) (1.5 g, 4.53 mmol) and tert butyl 2-(2-chloropyrimidin-5-yl)propionate(1.21 g, 4.99 mmol) in 15 mL of dimethyl sulfoxide was added potassium fluoride (1.32 g, 22.67 mmol) and TGME (744.4 mg, 4.53 mmol). The reaction solution was heated to 130° C. and stirred for 16 hours. The reaction solution was filtered, added with 50 mL of water and extracted with ethyl acetate. The organic phase was washed with saturated saline solution, dried over anhydrous sodium sulfate, filtered, concentrated, and purified by silica gel column chromatography to obtain compound tert butyl 2-(2-(((S)-1-(4-((8-chloro-1,7-naphthyridin-2-yl)amino) bicyclo[2.2.2]octan-1-yl)ethyl)amino)pyrimidin-5-yl)propionate (PA169-10)(1.15 g, 1.93 mmol, 42.5% yield) as a white solid. (ESI) m/z. [M+H]+ 537.2.

### 11. Synthesis of Compound 2-(2-(((S)-1-(4-((8-chloro-1,7-naphthyridin-2-yl) amino)bicyclo[2.2.2]octan-1-yl)ethyl)amino)pyrimidin-5-yl)propionic acid (PA169-11)

To a solution of compound tert butyl 2-(2-(((S)-1-(4-((8-chloro-1,7-naphthyridin-2-yl)amino) bicyclo[2.2.2]octan-1-yl)ethyl)amino)pyrimidin-5-yl)propionate (PA169-10) (1.1 g, 2.05 mmol) in 10 mL of dichloromethane was added dropwise 5 mL of trifluoroacetic acid at room temperature under stirring; the reaction solution was stirred for another 2 hours and then concentrated to obtain 1.00 g crude product of 2-(2-(((S)-1-(4-((8-chloro-1,7-naphthyridin-2-yl)amino)bicyclo[2.2.2]octan-1-yl)ethyl)amin o)pyrimidin-5-yl)propionic acid (PA169-11) as a brown solid. (ESI) m/z. [M+H]+ 481.20.

### 12. Synthesis of Compound 2-(2-(((S)-1-(4-((8-chloro-1,7-naphthyridin-2-yl) amino)bicyclo[2.2.2]octan-1-yl)ethyl)amino)pyrimidin-5-yl)-N-(oxetan-3-yl)propanamide (PA169)

To a solution of compound 2-(2-(((S)-1-(4-((8-chloro-1,7-naphthyridin-2-yl) amino)bicyclic[2.2.2]octan-1-yl)ethyl)amino)pyrimidin-5-yl)propionic acid (PA169-11) (1 g, 2.08 mmol) in 10 mL of DMF was added diisopropylethylamine (1.34 g, 10.40 mmol), oxetan-3-amine (303.9 mg, 4.16 mmol) and HATU (948.6 mg, 2.49 mmol) under stirring at room temperature. The reaction was stirred for 1 hour until the reaction was complete. The reaction solution was filtered, concentrated and subjected to preparative HPLC purification to obtain compound 2-(2-(((S)-1-(4-((8-chloro-1,7-naphthyridin-2-yl) amino)bicyclo[2.2.2]octan-1-yl)ethyl)amino)pyrimidin-5-yl)-N-(oxetan-3-yl)propanamide (PA169) (620.0 mg, 1.12 mmol, 53.9% yield) as a white solid. (ESI) m/z. [M+H]+ 536.1.

### 13. Preparation of Diastereoisomers PA166 and PA207

Compound 2-(2-(((S)-1-(4-((8-chloro-1,7-naphthyridin-2-yl) amino)bicyclo[2.2.2]octan-1-yl)ethyl)amino)pyrimidin-5-yl)-N-(oxetan-3-yl)propanamide (PA169) (620.0 mg, 1.16 mmol) was separated by SFC chiral column to obtain two diastereomer compounds PA166 (205.0 mg, 372.3 μmol, 32.2% yield) and PA207 (219.2 mg, 389.8 μmol, 33.7% yield) as white solids. The absolute configurations of the amide ortho chiral center of these two monomer compounds were temporarily randomly assigned.

PA166: ^{1H}NMR (400 MHz, DMSO-d6) δ (ppm) 8.75 (d, 1H), 8.15 (s, 2H), 7.96 (d, 1H), 7.87 (d, 1H), 7.53 (d, 1H), 7.18 (s, 1H), 7.02 (d, 1H), 6.74 (d, 1H), 4.79-4.64 (m, 3H), 4.41 (t, 1H), 4.36 (t, 1H), 3.98-3.89 (m, 1H), 3.42 (t, 1H), 2.11-2.05 (m, 6H), 1.62-1.51 (m, 6H), 1.30 (d, 3H), 1.01 (d, 3H); (ESI) m/z. [M+H]+ 536.0.

PA207: ¹H NMR (400 MHz, DMSO-d6) δ (ppm) 8.76 (d, 1H), 8.15 (s, 2H), 7.95 (d, 1H), 7.86 (d, 1H), 7.53 (d, 1H), 7.18 (s, 1H), 7.02 (d, 1H), 6.75 (d, 1H), 4.79-4.64 (m, 3H), 4.41 (t, 1H), 4.35 (t, 1H), 3.99-3.90 (m, 1H), 3.41 (q, 1H), 2.10-2.06 (m, 6H), 1.64-1.50 (m, 6H), 1.30 (d, 3H), 1.00 (d, 3H); (ESI) m z. [M+H]+ 536.0.

The following compounds were prepared using similar methods:

| Number | Structure | Compound characterization data |
|---|---|---|
| PA001 | 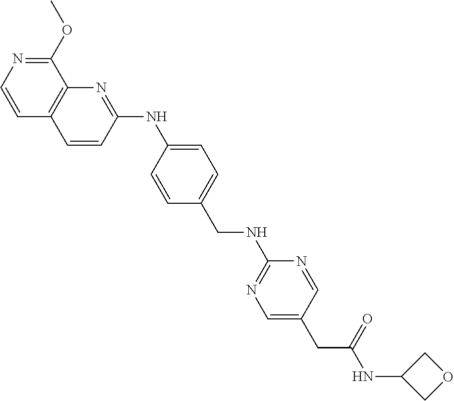 [SMILES: COc1nccc2ccc(Nc3ccc(CNc4ncc(CC(=O)NC5COC5)cn4)cc3)nc12] | ¹H NMR (400 MHz, DMSO-d6) δ (ppm) 9.57 (s, 1H), 8.79 (d, 1H), 8.14 (s, 2H), 8.01 (d, 1H), 7.93 (d, 2H), 7.84 (d, 1H), 7.56 (t, 1H), 7.28 − 7.21 (m, 4H), 4.76 (q, 1H), 4.69 (t, 2H), 4.45 (d, 2H), 4.40 (t, 2H), 4.03 (s, 3H), 3.25 (s, 2H). (ESI) m/z. [M + H]+ 472.3 |
| | PA001 | |
| PA007 | 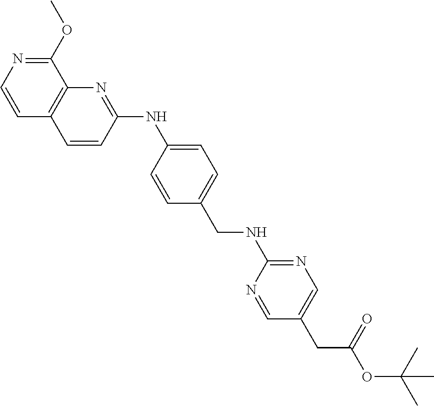 [SMILES: COc1nccc2ccc(Nc3ccc(CNc4ncc(CC(=O)OC(C)(C)C)cn4)cc3)nc12] | ¹H NMR (400 MHz, DMSO-d6) δ (ppm) 9.58 (s, 1H), 8.16 (s, 2H), 8.01 (d, 1H), 7.94 (d, 2H), 7.84 (d, 1H), 7.60 (t, 1H), 7.28 − 7.21 (m, 4H), 4.46 (d, 2H), 4.03 (s, 3H), 3.39 (s, 2H), 1.40 (s, 9H). (ESI) m/z. [M + H]+ 473.3 |
| | PA007 | |
| PA010 | 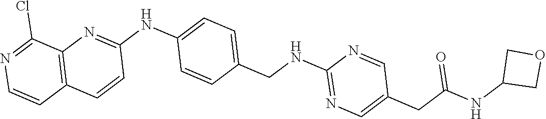 [SMILES: O=C(Cc1cnc(NCc2ccc(Nc3ccc4ccnc(Cl)c4n3)cc2)nc1)NC1COC1] | ¹H NMR (400 MHz, DMSO-d6) δ (ppm) 9.93 (s, 1H), 8.81 (br d, 1H), 8.11-8.18 (m, 4H), 8.06 (d, 2H), 7.71 (d, 1H), 7.59 (br t, 1H), 7.28-7.35 (m, 3H), 4.76 (td, 1H), 4.61-4.72 (m, 2H), 4.47 (br d, 2H), 4.41 (t, 2H), 3.26 (s, 2H); (ESI) m/z. [M + H]+ 476.1 |
| | PA010 | |
| PA033 | 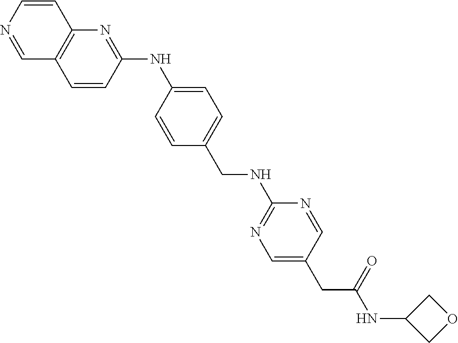 [SMILES: O=C(Cc1cnc(NCc2ccc(Nc3ccc4cnccc4n3)cc2)nc1)NC1COC1] | ¹H NMR (400 MHz, DMSO-d6) δ (ppm) 9.75 (s, 1H), 8.94 (s, 1H), 8.80 (d, 1H), 8.46 (d, 1H), 8.17 − 8.10 (m, 3H), 7.86 (d, 2H), 7.59 (t, 1H), 7.48 (d,1H), 7.28 (d, 2H), 7.10 (d, 1H), 4.72 (m, 3H), 4.48 − 4.36 (m, 4H), 3.25 (s, 2H). (ESI) m/z. [M + H]+ 442.1 |
| | PA033 | |
| PA034 | 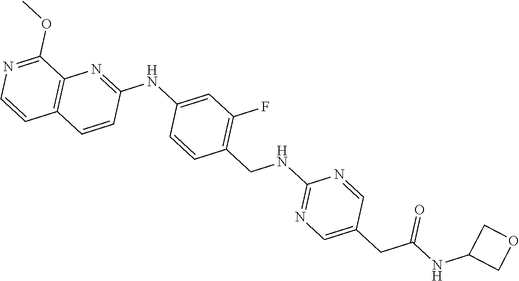 [SMILES: COc1nccc2ccc(Nc3ccc(CNc4ncc(CC(=O)NC5COC5)cn4)c(F)c3)nc12] | ¹H NMR (400 MHz, DMSO-d6) δ (ppm) 9.84 (s, 1H), 8.87 − 8.74 (m, 1H), 8.39 (d, 1H), 8.18 (s, 2H), 8.09 (d, 1H), 7.90 (d, 1H), 7.57 (t, 1H), 7.42 (d, 1H), 7.32 − 7.24 (m, 3H), 4.82 − 4.74 (m, 1H), 4.73 − 4.66 (m, 2H), 4.52 (d, 2H), 4.42 (dd, 6.4 Hz, 2H), 4.07 (s, 3H), 3.27 (s, 2H). (ESI) m/z. [M + H]+ 490.1 |
| | PA034 | |
| PA038 | 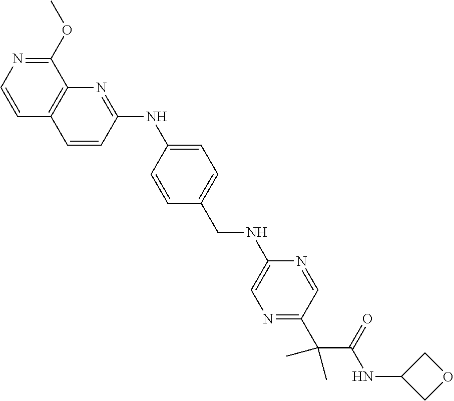 [SMILES: COc1nccc2ccc(Nc3ccc(CNc4cnc(C(C)(C)C(=O)NC5COC5)cn4)cc3)nc12] | ¹H NMR (400 MHz, DMSO-d6) δ (ppm) 9.58 (s, 1H), 8.21 (s, 2H), 8.12 (d, Hz, 1H), 8.02 (d, 1H), 7.94 (d, 2H), 7.84 (d, 1H), 7.64 (t, 1H), 7.28 (d, 2H), 7.21-7.26 (m, 2H), 4.71-4.78 (m, 1H), 4.62-4.71 (m, 2H), 4.46 (d, 2H), 4.41 (t, 2H), 4.03 (s, 3H), 1.43 (s, 6H) (ESI) m/z. [M + H]+ 500.3 |
| | PA038 | |
| PA039 | 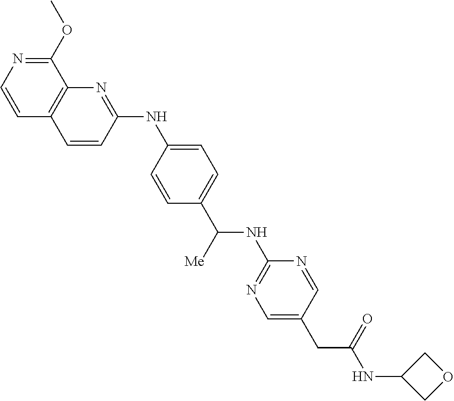 [SMILES: COc1nccc2ccc(Nc3ccc(C(C)Nc4ncc(CC(=O)NC5COC5)cn4)cc3)nc12] | ¹H NMR (400 MHz, DMSO-d6) δ (ppm) 9.56 (s, 1H), 8.78 (d, 1H), 8.11 (s, 2H), 8.01 (d, 1H), 7.91 (d, 2H), 7.83 (d, 1H), 7.51 (d, 1H), 7.33 (d, 2H), 7.18-7.25 (m, 2H), 5.00-5.09 (m, 1H), 4.70-4.80 (m, 1H), 4.64-4.69 (m, 2H), 4.39 (t, 2H), 4.03 (s, 3H), 3.22 (s, 2H), 1.43 (d, 3H) (ESI) m/z. [M + H]+ 486.2 |
| | PA039 | |
| PA041 | 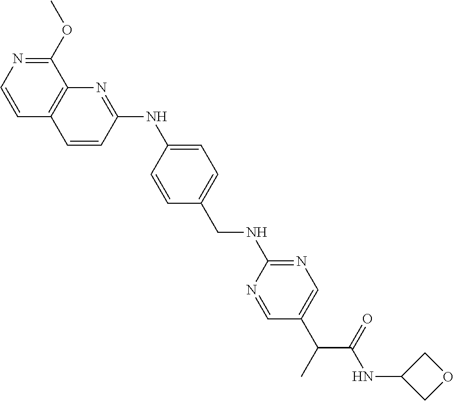 [SMILES: COc1nccc2ccc(Nc3ccc(CNc4ncc(C(C)C(=O)NC5COC5)cn4)cc3)nc12] | ¹H NMR (400 MHz, DMSO-d6) δ (ppm) 9.58 (s, 1H), 8.75 (d, 1H), 8.18-8.22 (m, 2H), 8.02 (d, 1H), 7.94 (d, 2H), 7.84 (d, 1H), 7.61 (t, 1H), 7.22-7.29 (m, 4H), 4.64-4.78 (m, 3H), 4.45 (d, 2H), 4.38-4.43 (m, 1H), 4.34 (t, 1H), 4.03 (s, 3H), 3.39-3.46 (m, 1H), 1.30 (d, 3H) (ESI) m/z. [M + H]+ 486.2 |
| | PA041 | |
| PA042 | 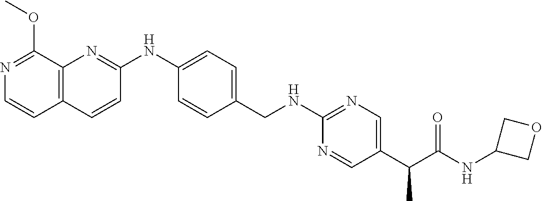 [SMILES: COc1nccc2ccc(Nc3ccc(CNc4ncc([C@H](C)C(=O)NC5COC5)cn4)cc3)nc12] | ¹H NMR (400 MHz, DMSO-d6) δ (ppm) 9.57 (s, 1H), 8.73 (br d, 1H), 8.20 (s, 2H), 8.02 (d, 1H), 7.93 (br d, 2H), 7.80-7.89 (m, 1H), 7.58 (br t, 1H), 7.16-7.35 (m, 4H), 4.55-4.80 (m, 3H), 4.45 (br d, 2H), 4.39-4.42 (m, 1H), 4.34 (br t, 1H), 4.03 (s, 3H), 3.44-3.47 (m, 1H), 1.30 (br d, 3H) (ESI) m/z. [M + H]+ 486.2 |
| | PA042 | |
| PA043 | 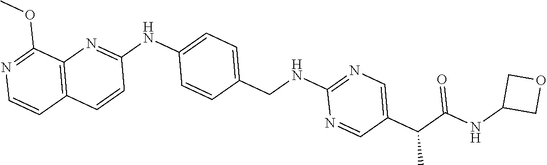 [SMILES: COc1nccc2ccc(Nc3ccc(CNc4ncc([C@@H](C)C(=O)NC5COC5)cn4)cc3)nc12] | ¹H NMR (400 MHz, DMSO-d6) δ (ppm) 9.57 (s, 1H), 8.73 (br d, 1H), 8.20 (s, 2H), 8.02 (d, 1H), 7.93 (br d, 2H), 7.84 (d, 1H), 7.58 (br t, 1H), 7.16-7.34 (m, 4H), 4.55-4.80 (m, 3H), 4.45 (br d, 2H), 4.37-4.43 (m, 1H), 4.30-4.37 (m, 1H), 4.03 (s, 3H), 3.46 (br s, 1H), 1.30 (br d, 3H) (ESI) m/z. [M + H]+ 486.2 |
| | PA043 | |
| PA044 | 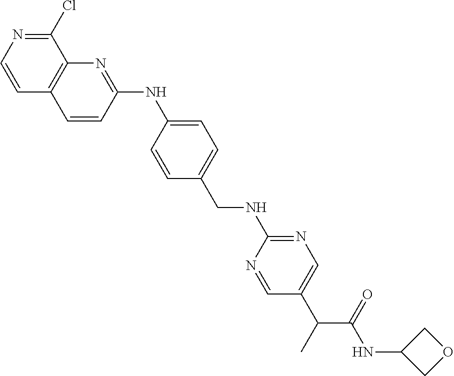 [SMILES: CC(C(=O)NC1COC1)c1cnc(NCc2ccc(Nc3ccc4ccnc(Cl)c4n3)cc2)nc1] | ¹H NMR (400 MHz, DMSO-d6) δ (ppm) 9.92 (s, 1H), 8.75 (d, 1H), 8.20 (s, 2H), 8.12-8.18 (m, 2H), 8.06 (d, 2H), 7.71 (d, 1H), 7.62 (t, 1H), 7.32 (t, 3H), 4.64-4.77 (m, 3H), 4.46 (d, 2H), 4.37-4.43 (m, 1H), 4.34 (t, 1H), 3.42-3.46 (m, 1H), 1.30 (d, 3H) (ESI) m/z. [M + H]+ 490.2 |
| | PA044 | |
| PA045 | 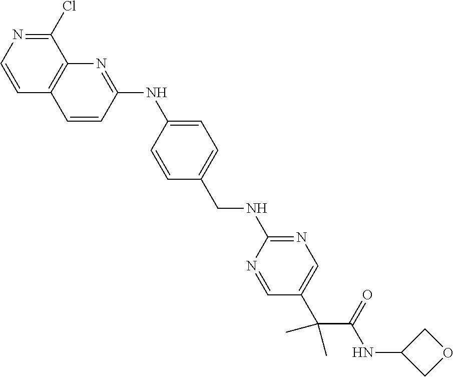 [SMILES: CC(C)(C(=O)NC1COC1)c1cnc(NCc2ccc(Nc3ccc4ccnc(Cl)c4n3)cc2)nc1] | ¹H NMR (400 MHz, DMSO-d6) δ (ppm) 9.92 (s, 1H), 8.20 (s, 2H), 8.08-8.18 (m, 3H), 8.06 (d, 2H), 7.71 (d, 1H), 7.63 (t, 1H), 7.33 (dd, 3H), 4.69-4.79 (m, 1H), 4.62-4.69 (m, 2H), 4.48 (d, 2H), 4.35-4.44 (m, 2H), 1.43 (s, 6H) (ESI) m/z. [M + H]+ 504.2 |
| | PA045 | |
| PA046 | 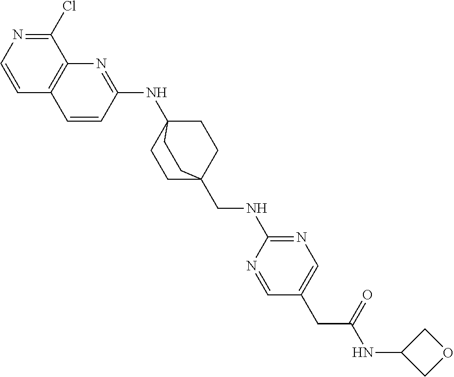 [SMILES: O=C(Cc1cnc(NCC23CCC(Nc4ccc5ccnc(Cl)c5n4)(CC2)CC3)nc1)NC1COC1] | 1H NMR (400 MHz, DMSO-d6) δ (ppm) 8.78 (br d, 1H), 8.11 (s, 2H), 7.96 (d, 1H), 7.87 (d, 1H), 7.53 (d, 1H), 7.15 (s, 1H), 7.02 (d, 1H), 6.92 (br t, 1H), 4.73-4.83 (m, 1H), 4.62-4.73 (m, 2H), 4.42 (t, 2H), 3.22-3.27 (m, 2H), 3.13 (br d, 2H), 2.01-2.18 (m, 6H), 1.46-1.65 (m, 6H) (ESI) m/z. [M + H]+ 508.2 |
| | PA046 | |
| PA048 | 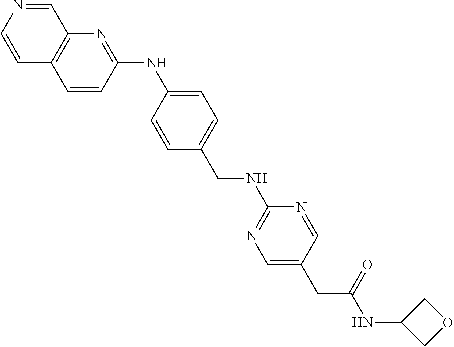 [SMILES: O=C(Cc1cnc(NCc2ccc(Nc3ccc4ccncc4n3)cc2)nc1)NC1COC1] | ¹H NMR (400 MHz, DMSO-d6) δ (ppm) 9.62 (s, 1H), 8.99 (s, 1H), 8.79 (d, 1H), 8.32 (d, 1H), 8.15 (s, 2H), 8.08 (d, 1H), 7.93-7.86 (m, 2H), 7.64 (d, 1H), 7.57 (t, 1H), 7.34-7.23 (m, 3H), 4.82-4.72 (m, 1H), 4.70 (d, 2H), 4.46 (d, 2H), 4.44-4.38 (m, 2H), 3.26 (s, 2H). (ESI) m/z. [M + H]+ 442.2 |
| | PA048 | |
| PA051 | 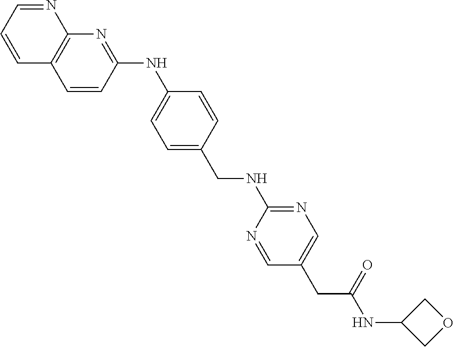 [SMILES: O=C(Cc1cnc(NCc2ccc(Nc3ccc4cccnc4n3)cc2)nc1)NC1COC1] | ¹H NMR (400 MHz, DMSO-d6) δ (ppm) 9.82 (s, 1H), 8.80 − 8.78 (m, 2H), 8.28 (d, 1H), 8.14 − 8.12 (m, 3H), 7.91 (d, 2H), 7.58 (t, 1H), 7.38 − 7.35 (m, 1H), 7.30 (d, 2H), 7.16-7.14 (m, 1H), 4.77-4.75 (m, 1H), 4.70-4.67 (m, 2H), 4.47-4.39 (m, 4H), 3.25 (s, 2H). (ESI) m/z. [M + H]+ 442.0 |
| | PA051 | |
| PA52 | 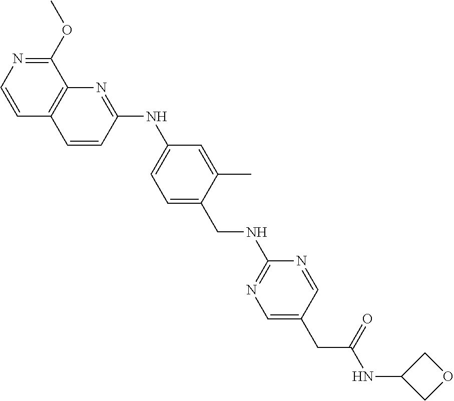 [SMILES: COc1nccc2ccc(Nc3ccc(CNc4ncc(CC(=O)NC5COC5)cn4)c(C)c3)nc12] | ¹H NMR (400 MHz, DMSO-d6) δ (ppm) 9.52 (s, 1H), 8.80 (d, 1H), 8.15 (s, 2H), 8.02-7.99 (m, 1H), 7.86-7.83 (m, 2H), 7.80-7.77 (m, 1H), 7.45 (t, 4.0 Hz, 1H), 7.24-7.22 (m, 2H), 7.19-7.17 (m, 1H), 4.79-4.74 (m, 1H), 4.71-4.67 (m, 2H), 4.43-4.39 (m, 4H), 4.03 (s, 3H), 3.26 (s, 2H), 2.34 (s, 3H). (ESI) m/z. [M + H]+ 486.2 |
| | PA052 | |
| PA053 | 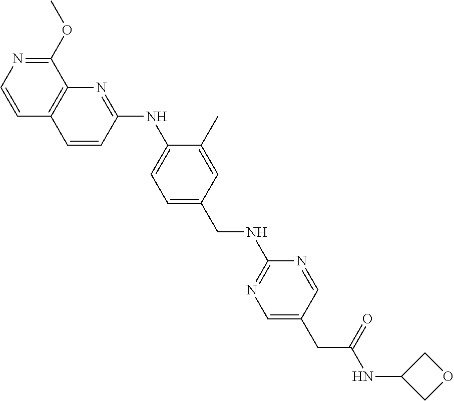 [SMILES: COc1nccc2ccc(Nc3ccc(CNc4ncc(CC(=O)NC5COC5)cn4)cc3C)nc12] | ¹H NMR (400 MHz, DMSO-d6) δ (ppm) 8.84-8.72 (m, 2H), 8.14 (s, 2H), 7.97 (d, 1H), 7.78 (d, 1H), 7.69 (d, 1H), 7.60 (t, 1H), 7.22-7.10 (m, 4H), 4.72 (dt, 7.1 Hz, 3H), 4.50-4.37 (m, 4H), 3.94 (s, 3H), 3.25 (s, 2H), 2.22 (s, 3H). (ESI) m/z. [M + H]+ 486.2 |
| | PA053 | |
| PA054 | 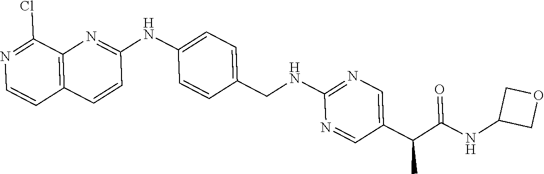 | ¹H NMR (400 MHz, DMSO-d6) δ (ppm) 9.93 (s, 1H), 8.75 (d, 1H), 8.20 (s, 2H), 8.11-8.18 (m, 2H), 8.06 (d, 2H), 7.71 (d, 1H), 7.61 (t, 1H), 7.32 (t, 3H), 4.63-4.78 (m, 3H), 4.46 (d, 2H), 4.37-4.44 (m, 1H), 4.34 (t, 1H), 3.41-3.46 (m, 1H), 1.30 (d, 3H) (ESI) m/z. [M + H]+ 486.2 |
| | PA054 | |
| PA055 | 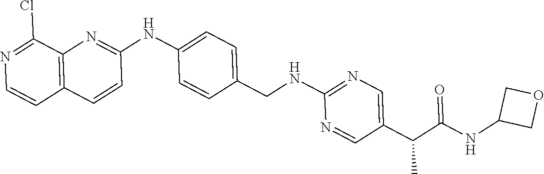 | ¹H NMR (400 MHz, DMSO-d6) δ (ppm) 9.93 (s, 1H), 8.75 (d, 1H), 8.20 (s, 2H), 8.12-8.18 (m, 2H), 8.06 (d, 2H), 7.71 (d, 1H), 7.61 (t, 1H), 7.32 (t, 3H), 4.63-4.77 (m, 3H), 4.46 (d, 2H), 4.36-4.44 (m, 1H), 4.34 (t, 1H), 3.42-3.50 (m, 1H), 1.30 (d, 3H) (ESI) m/z. [M + H]+ 486.2 |
| | PA055 | |
| PA056 | 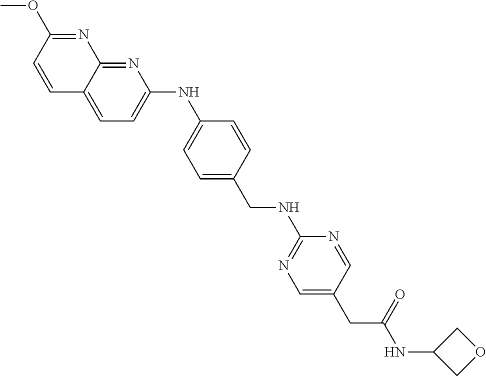 | ¹H NMR (400 MHz, DMSO-d6) δ (ppm) 8.80 (br d, 1H), 8.50 (s, 1H), 8.14 (s, 2H), 7.99 (d, 1H), 7.65 (d, 1H), 7.58 (t, 1H), 7.27 (d, 2H), 7.22 (d, 1H), 7.15 (d, 2H), 7.09 (dd, 1H), 6.68 (d, 1H), 4.72-4.81 (m, 1H), 4.66-4.71 (m, 2H), 4.44 (br d, 2H), 4.40 (t, 2H), 3.90 (s, 3H), 3.25 (s, 2H) (ESI) m/z. [M + H]+ 493.1 |
| | PA056 | |
| PA058 | 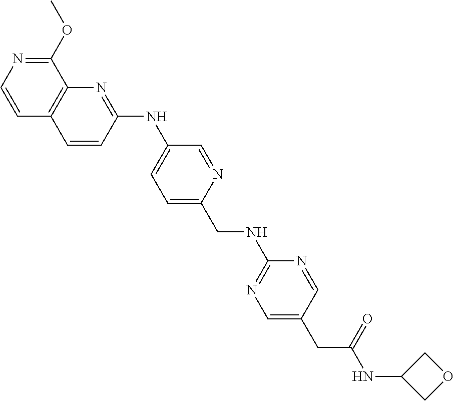 [SMILES: COc1nccc2ccc(Nc3ccc(CNc4ncc(CC(=O)NC5COC5)cn4)nc3)nc12] | ¹H NMR (400 MHz, DMSO-d6) δ (ppm) 9.87-9.78 (m, 1H), 9.15 (d, 1H), 8.82 (d, 1H), 8.40 (dd, 1H), 8.15 (s, 2H), 8.07 (d, 1H), 7.87 (d, 1H), 7.56 (t, 1H), 7.32-7.22 (m, 3H), 4.80-4.72 (m, 1H), 4.71- 4.63 (m, 2H), 4.54 (d, 2H), 4.40 (t, 2H), 4.04 (s, 3H), 3.26 (s, 2H). (ESI) m/z. [M + H]+ 473.2 |
| | PA058 | |
| PA061 | 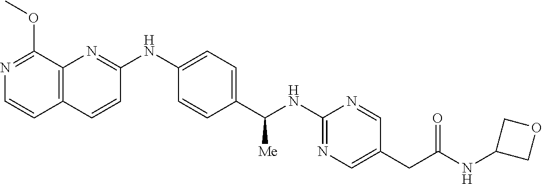 [SMILES: COc1nccc2ccc(Nc3ccc([C@H](C)Nc4ncc(CC(=O)NC5COC5)cn4)cc3)nc12] | ¹H NMR (400 MHz, DMSO-d6) δ (ppm) 9.56 (s, 1H), 8.78 (d, 1H), 8.11 (s, 2H), 8.01 (d, 1H), 7.91 (d, 2H), 7.83 (d, 1H), 7.51 (d, 1H), 7.33 (d, 2H), 7.18-7.25 (m, 2H), 5.00-5.09 (m, 1H), 4.70-4.80 (m, 1H), 4.64-4.69 (m, 2H), 4.39 (t, 2H), 4.03 (s, 3H), 3.22 (s, 2H), 1.43 (d, 3H) (ESI) m/z. [M + H]+ 486.1 |
| | PA061 | |
| PA062 | 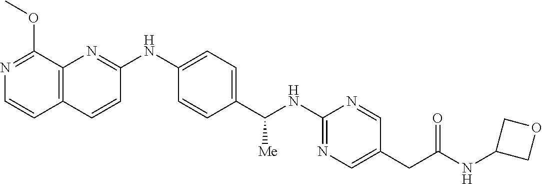 [SMILES: COc1nccc2ccc(Nc3ccc([C@@H](C)Nc4ncc(CC(=O)NC5COC5)cn4)cc3)nc12] | ¹H NMR (400 MHz, DMSO-d6) δ (ppm) 9.56 (s, 1H), 8.78 (d, 1H), 8.11 (s, 2H), 8.01 (d, 1H), 7.91 (d, 2H), 7.83 (d, 1H), 7.51 (d, 1H), 7.33 (d, 2H), 7.18-7.25 (m, 2H), 5.00-5.09 (m, 1H), 4.70-4.80 (m, 1H), 4.64-4.69 (m, 2H), 4.39 (t, 2H), 4.03 (s, 3H), 3.22 (s, 2H), 1.43 (d, 3H) (ESI) m/z. [M + H]+ 486.1 |
| | PA062 | |
| PA063 | 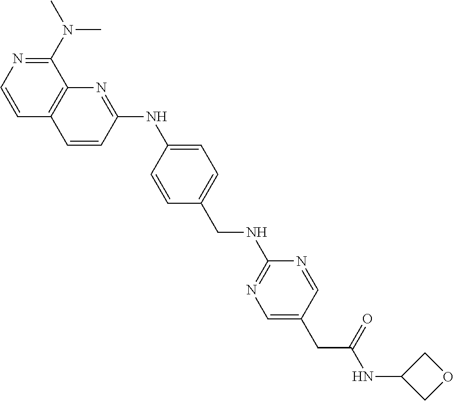 [SMILES: CN(C)c1nccc2ccc(Nc3ccc(CNc4ncc(CC(=O)NC5COC5)cn4)cc3)nc12] | ¹H NMR (400 MHz, DMSO-d6) δ (ppm) 9.40 (s, 1H), 8.75 (d, 1H), 8.11 (s, 2H), 7.91 (d, 1H), 7.81 (d, 1H), 7.73 (d, 2H), 7.51 (t, 1H), 7.24 (d, 2H), 7.12 (d, 1H), 6.96 (d, 1H), 4.80-4.67 (m, 1H), 4.65 (t, 2H), 4.42 (s, 2H), 4.37 (d, 2H), 3.22 (s, 2H), 3.18 (s, 6H). (ESI) m/z. [M + H]+ 485.1 |
| | PA063 | |
| PA064 | 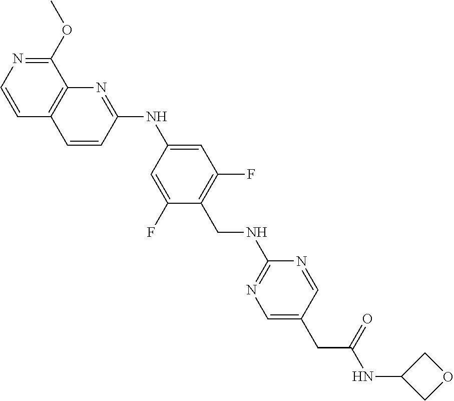 [SMILES: COc1nccc2ccc(Nc3cc(F)c(CNc4ncc(CC(=O)NC5COC5)cn4)c(F)c3)nc12] | ¹H NMR (400 MHz, DMSO-d6) δ (ppm) 10.00 (s, 1H), 8.79 (d, 1H), 8.17 (s, 2H), 8.13-8.11 (m, 1H), 7.91 (d, 1H), 7.82-7.79 (m, 2H), 7.32 (t, 1H), 7.29-7.24 (m, 2H), 4.81-4.73 (m, 1H), 4.71 − 4.67 (m, 2H), 4.48 (d, 2H), 4.41 (t, 2H), 4.06 (s, 3H), 3.26 (s, 2H). (ESI) m/z. [M + H]+ 508.1 |
| | PA064 | |
| PA066 | 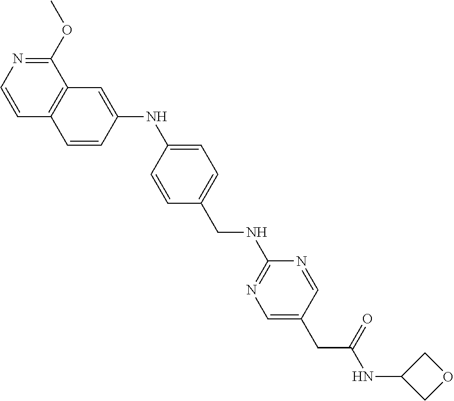 [SMILES: COc1nccc2ccc(Nc3ccc(CNc4ncc(CC(=O)NC5COC5)cn4)cc3)cc12] | ¹H NMR (400 MHz, DMSO-d6) δ (ppm) 8.76 (d, 1H), 8.47 (s, 1H), 8.11 (s, 2H), 7.73-7.67 (m, 2H), 7.60 (d, 1H), 7.55-7.49 (m, 1H), 7.40 (dd, 1H), 7.22 (d, 2H), 7.18 (d, 1H), 7.09 (d, 2H), 4.76-4.68 (m, 1H), 4.65 (t, 2H), 4.41 (d, 2H), 4.39-4.35 (m, 2H), 3.95 (s, 3H), 3.22 (s, 2H). (ESI) m/z. [M + H]+ 471.2 |
| | PA066 | |
| PA068 | 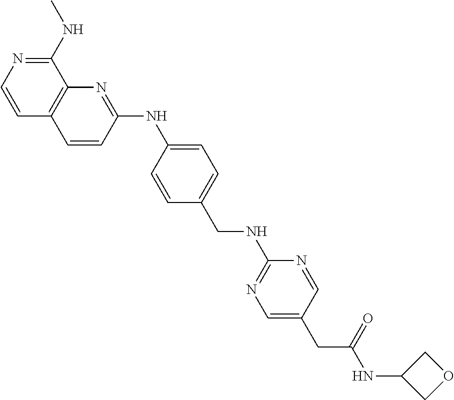 [SMILES: CNc1nccc2ccc(Nc3ccc(CNc4ncc(CC(=O)NC5COC5)cn4)cc3)nc12] | ¹H NMR (400 MHz, DMSO-d6) δ (ppm) 9.38 (s, 1H), 8.76 (d, 1H), 8.11 (s, 2H), 7.85 (d, 1H), 7.74-7.65 (m, 3H), 7.52 (t, 1H), 7.26 (d, 2H), 7.12 (d, 1H), 6.70 (d, 1H), 6.50 (q, 1H), 4.84-4.69 (m, 1H), 4.65 (t, 2H), 4.42 (d, 2H), 4.36 (d, 2H), 3.22 (s, 2H), 2.99 (d, 3H). (ESI) m/z. [M + H]+ 471.0 |
| | PA068 | |
| PA069 | 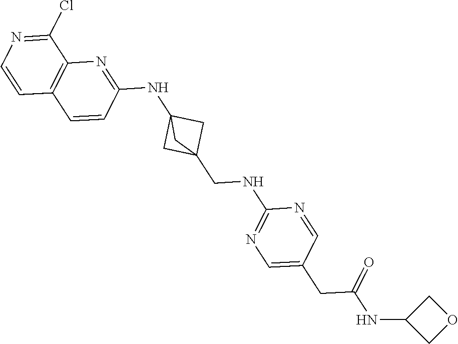 [SMILES: O=C(Cc1cnc(NCC23CC(Nc4ccc5ccnc(Cl)c5n4)(C2)C3)nc1)NC1COC1] | ¹H NMR (400 MHz, DMSO-d6) δ (ppm) 8.80 (br d, 1H), 8.19 (s, 1H), 8.13 (s, 2H), 7.93-8.05 (m, 2H), 7.60 (d, 1H), 7.04 (br t, 1H), 6.99 (br d, 1H), 4.73-4.81 (m, 1H), 4.66-4.73 (m, 2H), 4.41 (t, 2H), 3.47-3.58 (m, 2H), 3.21-3.29 (m, 2H), 2.10 (s, 6H) (ESI) m/z. [M + H]+ 466.2 |
| | PA069 | |
| PA070 | 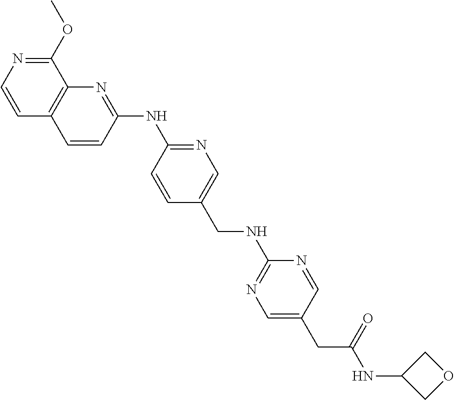 [SMILES: COc1nccc2ccc(Nc3ccc(CNc4ncc(CC(=O)NC5COC5)cn4)cn3)nc12] | ¹H NMR (400 MHz, DMSO-d6) δ (ppm) 10.26 (s, 1H), 8.80 (br d, 1H), 8.34 (d, 1H), 8.23 (d, 1H), 8.16 (s, 2H), 8.12 (d, 1H), 7.84-7.90 (m, 2H), 7.71 (dd, 1H), 7.62 (t, 1H), 7.27 (d, 1H), 4.71-4.80 (m, 1H), 4.65-4.71 (m, 2H), 4.44 (d, 2H), 4.40 (t, 2H), 4.04 (s, 3H), 3.26 (s, 2H) (ESI) m/z. [M + H]+ 473.2 |
| | PA070 | |
| PA072 | 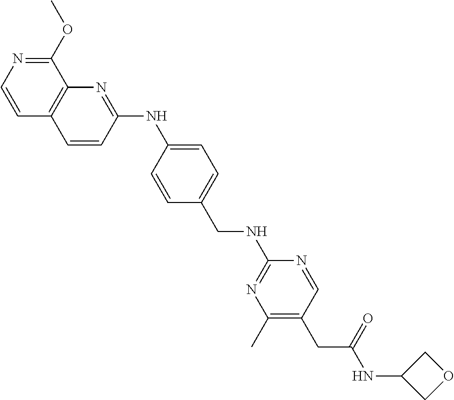 [SMILES: COc1nccc2ccc(Nc3ccc(CNc4ncc(CC(=O)NC5COC5)c(C)n4)cc3)nc12] | ¹H NMR (400 MHz, DMSO-d6) δ (ppm) 9.57 (s, 1H), 8.76 (d, 1H), 8.01 (d, 1H), 7.97 (s, 1H), 7.93 (d, 2H), 7.83 (d, 1H), 7.42 (br t, 1H), 7.26 (d, 2H), 7.23 (d, 1H), 7.22 (d, 1H), 4.72-4.83 (m, 1H), 4.66-4.72 (m, 2H), 4.45 (d, 2H), 4.41 (t, 2H), 4.03 (s, 3H), 3.30 (s, 2H), 2.22 (s, 3H) (ESI) m/z. [M + H]+ 486.1 |
| | PA072 | |
| PA073 | 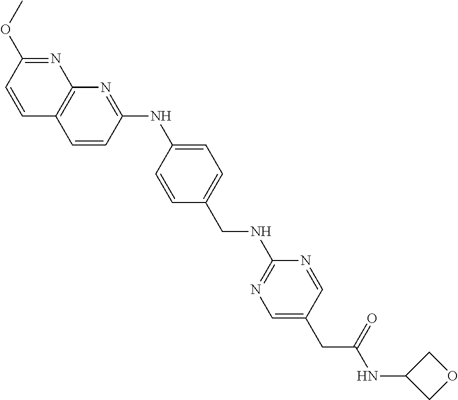 [SMILES: COc1ccc2ccc(Nc3ccc(CNc4ncc(CC(=O)NC5COC5)cn4)cc3)nc2n1] | ¹H NMR (400 MHz, DMSO-d6) δ (ppm) 9.88 − 9.78 (m, 1H), 8.82 (br d, 1H), 8.22 (s, 2H), 8.13 (t, 2H), 7.96 − 7.83 (m, 1H), 7.72 (br d, 2H), 7.34 (d, 2H), 6.97 (d, 1H), 6.88 (d, 1H), 4.83 − 4.73 (m, 1H), 4.73 − 4.66 (m, 2H), 4.51 (br s, 2H), 4.41 (t, 2H), 4.00 (s, 3H), 4.02 − 3.98 (m, 1H), 3.30 (s, 3H) (ESI) m/z. [M + H]+ 472.2 |
| | PA073 | |
| PA074 | 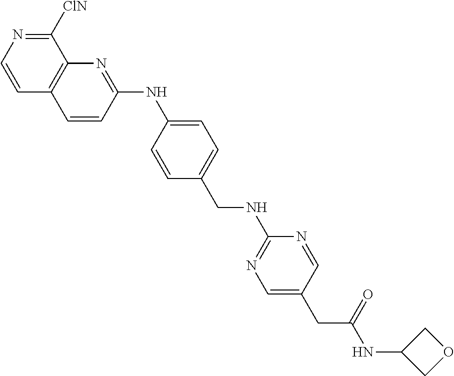 | ¹H NMR (400 MHz, DMSO-d6) δ (ppm) 10.15 (s, 1H), 8.81 (br d, 1H), 8.48 (d, 1H), 8.21 (d, 1H), 8.15 (s, 2H), 8.06 (d, 2H), 8.00 (d, 1H), 7.62 (t, 1H), 7.38 (d, 1H), 7.31 (d, 2H), 4.72-4.80 (m, 1H), 4.66-4.72 (m, 2H), 4.48 (d, 2H), 4.41 (t, 2H), 3.24-3.27 (m, 2H) (ESI) m/z. [M + H]+ 467.1 |
| | PA074 | |
| PA075 | 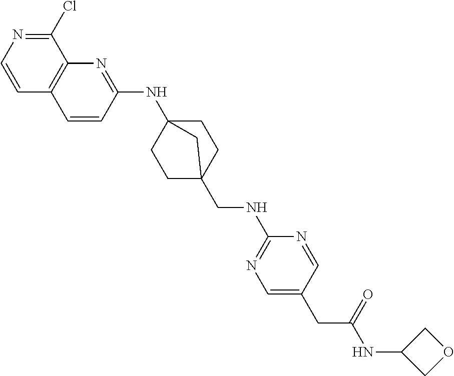 [SMILES: O=C(Cc1cnc(NCC23CCC(Nc4ccc5ccnc(Cl)c5n4)(CC2)C3)nc1)NC1COC1] | ¹H NMR (400 MHz, DMSO-d6) δ (ppm) 8.78 (br d, 1H), 8.11 (s, 2H), 7.96 (d, 1H), 7.88 (d, 1H), 7.82 (s, 1H), 7.54 (d, 1H), 6.98-7.08 (m, 2H), 4.65-4.83 (m, 3H), 4.40 (t, 2H), 3.45 (br d, 2H), 3.23 (s, 2H), 1.96-2.13 (m, 4H), 1.81 (s, 2H), 1.64-1.77 (m, 2H), 1.44 (br d, 2H) (ESI) m/z. [M + H]+ 494.1 |
| | PA075 | |
| PA076 | 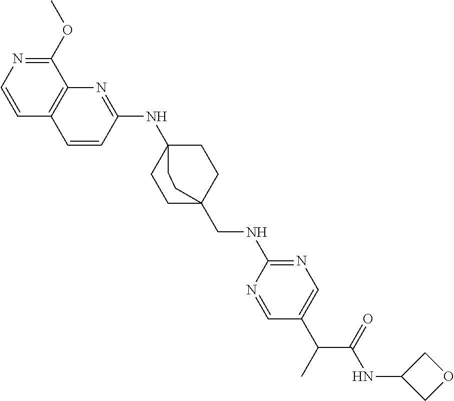 [SMILES: COc1nccc2ccc(NC34CCC(CNc5ncc(C(C)C(=O)NC6COC6)cn5)(CC3)CC4)nc12] | ¹H NMR (400 MHz, DMSO-d6) δ (ppm) 8.77 (d, 1H), 8.16 (s, 2H), 7.73 (d, 1H), 7.68 (d, 1H), 7.07 (d, 1H), 6.98 (t, 1H), 6.93 (d, 1H), 6.70 (s, 1H), 4.64-4.81 (m, 3H), 4.39-4.45 (m, 1H), 4.35 (t, 1H), 3.96 (s, 3H), 3.42-3.46 (m, 1H), 3.12 (br d, 2H), 1.98-2.07 (m, 6H), 1.43-1.57 (m, 6H), 1.30 (d, 3H) (ESI) m/z. [M + H]+ 518.2 |
| | PA076 | |
| PA077 | 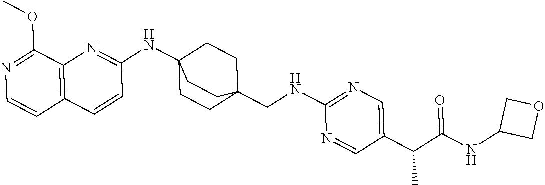 [SMILES: COc1nccc2ccc(NC34CCC(CNc5ncc([C@@H](C)C(=O)NC6COC6)cn5)(CC3)CC4)nc12] | ¹H NMR (400 MHz, DMSO-d6) δ (ppm) 8.75 (d, 1H), 8.16 (s, 2H), 7.73 (d, 1H), 7.69 (d, 1H), 7.07 (d, 1H), 6.98 (t, 1H), 6.93 (d, 1H), 6.70 (s, 1H), 4.63-4.81 (m, 3H), 4.39-4.44 (m, 1H), 4.35 (t, 1H), 3.96 (s, 3H), 3.42 (d, 1H), 3.12 (d, 2H), 1.98-2.09 (m, 6H), 1.45-1.58 (m, 6H), 1.30 (d, 3H) (ESI) m/z. [M + H]+ 518.2 |
| | PA077 | |
| PA078 | 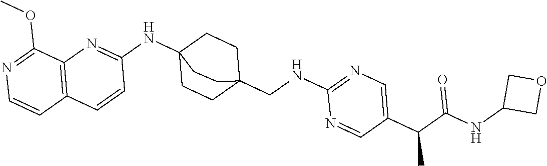 [SMILES: COc1nccc2ccc(NC34CCC(CNc5ncc([C@H](C)C(=O)NC6COC6)cn5)(CC3)CC4)nc12] | ¹H NMR (400 MHz, DMSO-d6) δ (ppm) 8.76 (d, 1H), 8.16 (s, 2H), 7.73 (d, 1H), 7.68 (d, 1H), 7.07 (d, 1H), 6.98 (t, 1H), 6.93 (d, 1H), 6.70 (s, 1H), 4.64-4.82 (m, 3H), 4.40-4.46 (m, 1H), 4.35 (t, 1H), 3.96 (s, 3H), 3.40-3.46 (m, 1H), 3.12 (d, 2H), 1.95-2.09 (m, 6H), 1.46-1.58 (m, 6H), 1.30 (d, 3H) (ESI) m/z. [M + H]+ 518.2 |
| | PA078 | |
| PA079 | 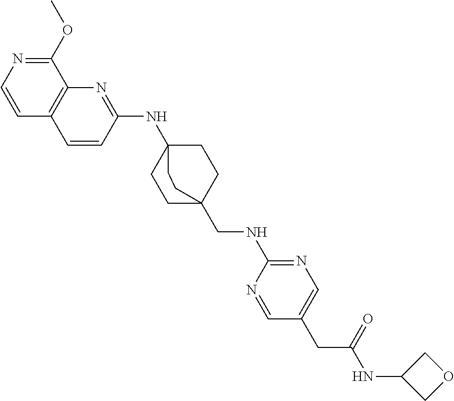 [SMILES: COc1nccc2ccc(NC34CCC(CNc5ncc(CC(=O)NC6COC6)cn5)(CC3)CC4)nc12] | ¹H NMR (400 MHz, CDCl₃) δ (ppm) 8.20 (s, 2H), 7.81 (d, 1H), 7.74 (d, 1H), 7.03 (d, 1H), 6.96 (d, 1H), 6.13 (br d, 1H), 5.20 (br t, 1H), 4.99-5.13 (m, 2H), 4.89-4.98 (m, 2H), 4.47 (t, 2H), 4.15 (s, 3H), 3.37 (s, 2H), 3.29 (d, 2H), 1.97-2.12 (m, 6H), 1.66-1.72 (m, 6H) (ESI) m/z. [M + H]+ 504.2 |
| | PA079 | |
| PA081 | 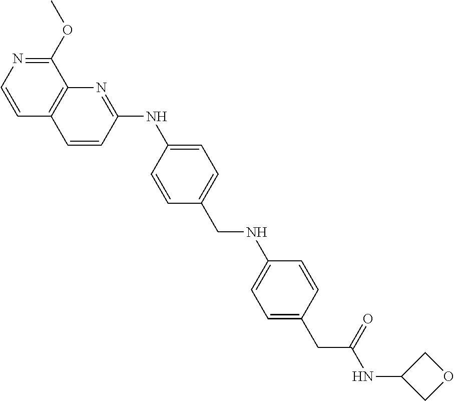 [SMILES: COc1nccc2ccc(Nc3ccc(CNc4ccc(CC(=O)NC5COC5)cc4)cc3)nc12] | ¹H NMR (400 MHz, DMSO-d6) δ (ppm) 9.59 (s, 1H), 8.66 (d, 1H), 8.02 (d, 1H), 7.97 (d, 2H), 7.85 (d, 1H), 7.31 (d, 2H), 7.21-7.27 (m, 2H), 6.94 (d, 2H), 6.53 (d, 2H), 6.08 (t, 1H), 4.65-4.78 (m, 3H), 4.39 (t, 2H), 4.20 (d, 2H), 4.04 (s, 3H), 3.21 (s, 2H) (ESI) m/z. [M + H]+ 470.2 |
| | PA081 | |
| PA082 | 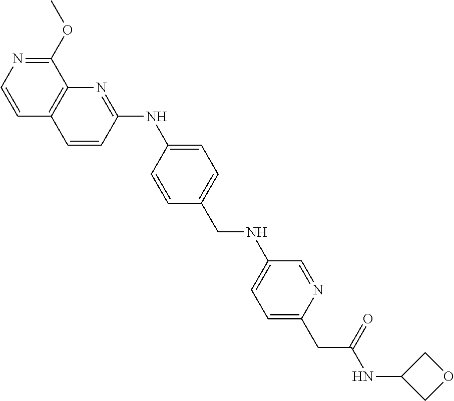 [SMILES: COc1nccc2ccc(Nc3ccc(CNc4ccc(CC(=O)NC5COC5)nc4)cc3)nc12] | ¹H NMR (400 MHz, DMSO-d6) δ (ppm) 9.61 (s, 1H), 8.73 (br d, 1H), 8.01 (dd, 3H), 7.82-7.93 (m, 2H), 7.33 (d, 2H), 7.20-7.28 (m, 2H), 6.99 (d, 1H), 6.89 (dd, 1H), 6.35 (t, 1H), 4.71-4.80 (m, 1H), 4.65-4.71 (m, 2H), 4.40 (t, 2H), 4.24 (d, 2H), 4.04 (s, 3H), 3.41 (s, 2H) (ESI) m/z. [M + H]+ 471.1 |
| | PA082 | |
| PA085 | 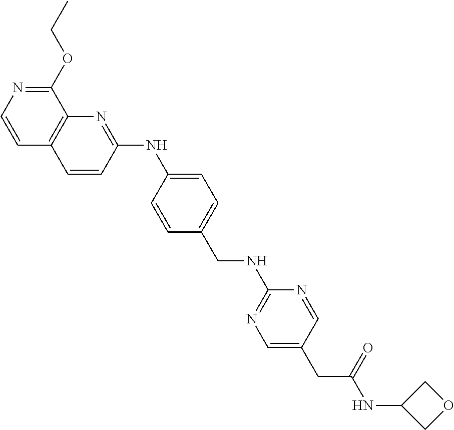 [SMILES: CCOc1nccc2ccc(Nc3ccc(CNc4ncc(CC(=O)NC5COC5)cn4)cc3)nc12] | ¹H NMR (400 MHz, DMSO-d6) δ (ppm) 8.87 (d, 1H), 8.42 (s, 2H), 8.05 (d, 1H), 7.92 (d, 1H), 7.86 (d, 1H), 7.31-7.26 (m, 3H), 6.39-6.33 (m, 2H), 5.39 (s, 2H), 4.84 (s, 2H), 4.81 − 4.72 (m, 1H), 4.68 (t, 2H), 4.49 (q, 2H), 4.40 (t, 2H), 3.40 (s, 2H), 1.45 (t, 3H). (ESI) m/z. [M + H]+ 486.2 |
| | PA085 | |
| PA089 | 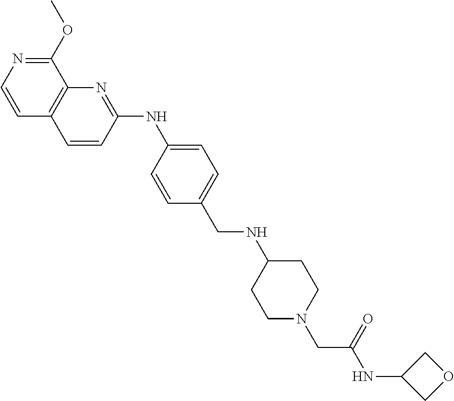 [SMILES: COc1nccc2ccc(Nc3ccc(CNC4CCN(CC(=O)NC5COC5)CC4)cc3)nc12] | ¹H NMR (400 MHz, DMSO-d6) δ (ppm) 9.54 (s, 1H), 8.34 (d, 1H), 7.98 (d, 1H), 7.92 (d, 2H), 7.81 (d, 1H), 7.25 (d, 2H), 7.23 − 7.17 (m, 2H), 4.76 (d, 1H), 4.68 − 4.58 (m, 2H), 4.45 (t, 2H), 4.00 (s, 3H), 3.65 (s, 2H), 2.84 (s, 2H), 2.73 (s, 2H), 2.36 (s, 1H), 2.01 (s, 2H), 1.76 (d, 2H), 1.37 (t, 2H). (ESI) m/z. [M + H]+ 477.1 |
| | PA089 | |
| PA106 | 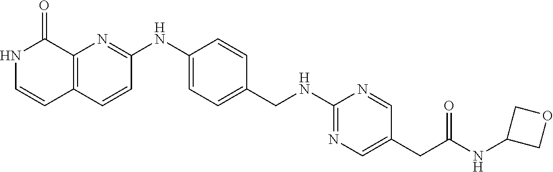 [SMILES: O=C(Cc1cnc(NCc2ccc(Nc3ccc4cc[nH]c(=O)c4n3)cc2)nc1)NC1COC1] | ¹H NMR (400 MHz, DMSO-d6) δ (ppm) 11.33 (s, 1H), 9.39 (s, 1H), 8.77 (d, 1H), 8.14 (s, 2H), 7.92 (d, 2H), 7.83 (d, 1H), 7.52 (t, 1H), 7.23 (d, 2H), 7.14 (d, 1H), 7.01 (d, 1H), 6.40 (d, 1H), 4.77 (dt, 1H), 4.69 (t, 2H), 4.48 − 4.37 (m, 4H), 3.25 (s, 2H). (ESI) m/z. [M + H]+ 458.2 |
| | PA106 | |
| PA110 | 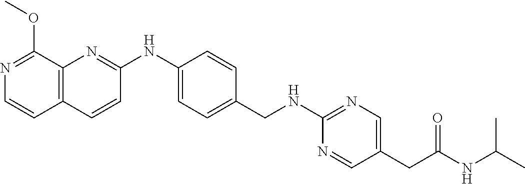 [SMILES: COc1nccc2ccc(Nc3ccc(CNc4ncc(CC(=O)NC(C)C)cn4)cc3)nc12] | ¹H NMR (400 MHz, DMSO-d6) δ (ppm) 9.57 (s, 1H), 8.14 (s, 2H), 8.01 (d, 1H), 7.92 (t, 3H), 7.84 (d, 1H), 7.53 (t, 1H), 7.28 − 7.21 (m, 4H), 4.46 (d, 2H), 4.03 (s, 3H), 3.79 (h, 1H), 3.18 (s, 2H), 1.04 (d, 6H). (ESI) m/z. [M + H]+ 458.2 |
| | PA110 | |
| PA112 | 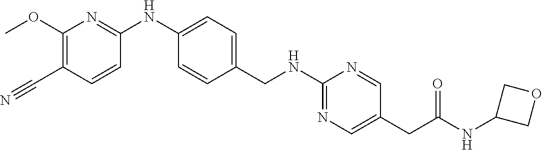 [SMILES: COc1nc(Nc2ccc(CNc3ncc(CC(=O)NC4COC4)cn3)cc2)ccc1C#N] | ¹H NMR (400 MHz, DMSO-d6) δ (ppm) 9.72 (s, 1H), 8.77 (s, 1H), 8.09 (s, 2H), 7.74 (d, 1H), 7.54 (d, 3H), 7.22 (d, 2H), 6.38 (d, 1H), 4.77 − 4.69 (m, 1H), 4.66 (q, 2H), 4.38 (dt, 4H), 3.91 (s, 3H), 3.21 (s, 2H). (ESI) m/z. [M + H]+ 446.0 |
| | PA112 | |
| PA113 | 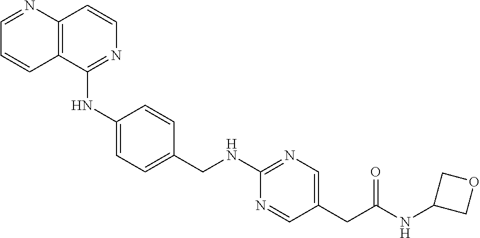 [SMILES: O=C(Cc1cnc(NCc2ccc(Nc3nccc4ncccc34)cc2)nc1)NC1COC1] | ¹H NMR (400 MHz, DMSO-d6) δ (ppm) 9.29 (s, 1H), 8.96 (dd, 1H), 8.89 (m, 1H), 8.77 (m, 1H), 8.12 (m, 3H), 7.71 (d, 2H), 7.61 − 7.51 (m, 2H), 7.24 (m, 2H), 7.16 (d, 1H), 4.74 (m, 1H), 4.65 (t, 2H), 4.43 (s, 2H), 4.38 (t, 2H), 3.22 (s, 2H). (ESI) m/z. [M + H]+ 442.0 |
| | PA113 | |
| PA122 | 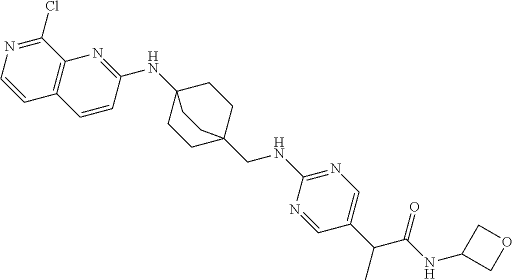 [SMILES: CC(C(=O)NC1COC1)c1cnc(NCC23CCC(Nc4ccc5ccnc(Cl)c5n4)(CC2)CC3)nc1] | ¹H NMR (400 MHz, DMSO-d6) δ (ppm) 8.73 (d, 1H), 8.15 (s, 2H), 7.95 (d, 1H), 7.86 (d, 1H), 7.53 (d, 1H), 7.16 (s, 1H), 7.01 (d, 1H), 6.97 (t, 1H), 4.68-4.79 (m, 2H), 4.62-4.68 (m, 1H), 4.41 (t, 1H), 4.34 (t, 1H), 3.41 (q, 1H), 3.12 (d, 2H), 2.04-2.14 (m, 6H), 1.49-1.59 (m, 6H), 1.29 (d, 3H) (ESI) m/z. [M + H]+ 522.1 |
| | PA122 | |
| PA123 | 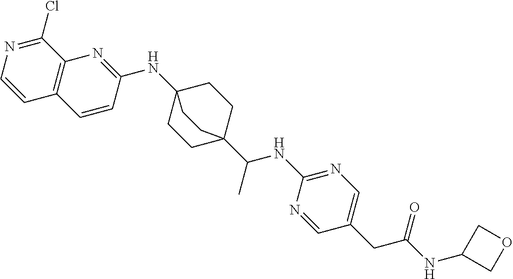 [SMILES: CC(Nc1ncc(CC(=O)NC2COC2)cn1)C12CCC(Nc3ccc4ccnc(Cl)c4n3)(CC1)CC2] | ¹H NMR (400 MHz, DMSO-d6) δ (ppm) 8.80 (br d, 1H), 8.09 (s, 2H), 7.95 (d, 1H), 7.86 (d, 1H), 7.53 (d, 1H), 7.18 (s, 1H), 7.01 (d, 1H), 6.70 (d, 1H), 4.81 − 4.74 (m, 1H), 4.73 − 4.62 (m, 2H), 4.41 (t, 2H), 4.00 − 3.87 (m, 1H), 3.23 (s, 2H), 2.07 (br t, 6H), 1.63 − 1.45 (m, 6H), 1.01 (d, 3H) (ESI) m/z. [M + H]+ 522.3 |
| | PA123 | |
| PA124 | 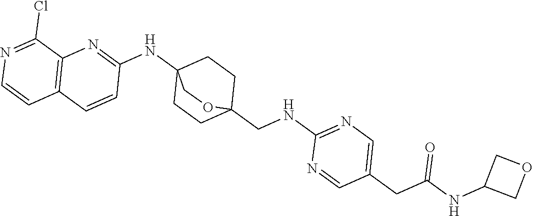 [SMILES: O=C(Cc1cnc(NCC23CCC(Nc4ccc5ccnc(Cl)c5n4)(CC2)CO3)nc1)NC1COC1] | ¹H NMR (400 MHz, DMSO-d6) δ (ppm) 8.80 (br d, 1H), 8.12 (s, 2H), 7.99 (d, 1H), 7.92 (d, 1H), 7.57 (d, 1H), 7.31 (s, 1H), 7.04 (d, 1H), 6.77 (t, 1H), 4.72-4.80 (m, 1H), 4.66-4.72 (m, 2H), 4.41 (t, 2H), 4.23 (s, 2H), 3.25 (s, 2H), 2.30-2.47 (m, 4H), 1.95-2.05 (m, 2H), 1.82-1.91 (m, 2H), 1.70-1.80 (m, 2H) (ESI) m/z. [M + H]+ 510.1 |
| | PA124 | |
| PA151 | 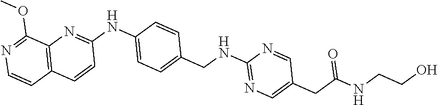 [SMILES: COc1nccc2ccc(Nc3ccc(CNc4ncc(CC(=O)NCCO)cn4)cc3)nc12] | ¹H NMR (400 MHz, DMSO-d6) δ (ppm) 9.56 (s, 1H), 8.16 (s, 2H), 8.01 (d, 2H), 7.94 (d, 2H), 7.84 (d, 1H), 7.52 (t, 1H), 7.32 − 7.19 (m, 4H), 4.67 (t, 1H), 4.47 (d, 2H), 4.04 (s, 3H), 3.40 (q, 2H), 3.24 (s, 2H), 3.12 (q, 2H). (ESI) m/z. [M + H]+ 460.2 |
| | PA151 | |
| PA152 | 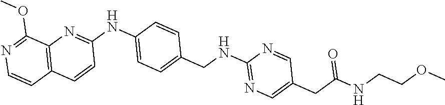 | ¹H NMR (400 MHz, DMSO-d6) δ (ppm) 9.57 (s, 1H), 8.15 (s, 2H), 8.10 (t, 1H), 8.01 (d, 1H), 7.94 (d, 2H), 7.84 (d, 1H), 7.53 (t, 1H), 7.28 (s, 1H), 7.24 (dd, 3H), 4.46 (d, 2H), 4.04 (s, 3H), 3.32 (d, 2H), 3.23 (s, 3H), 3.21 − 3.16 (m, 4H). (ESI) m/z. [M + H]+ 474.2 |
| | PA152 | |
| PA153 | 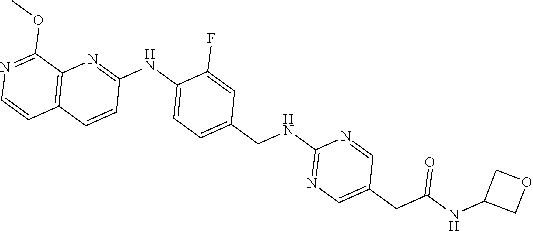 [SMILES: COc1nccc2ccc(Nc3ccc(CNc4ncc(CC(=O)NC5COC5)cn4)cc3F)nc12] | ¹H NMR (400 MHz, DMSO-d6) δ (ppm) 9.22 (s, 1H), 8.79 (d, 1H), 8.65 (t, 1H), 8.16 (s, 2H), 8.05 (d, 1H), 7.86 (d, 1H), 7.63 (t, 1H), 7.44 (d, 1H), 7.24 (d, 1H), 7.19 − 7.13 (m, 2H), 4.79 − 4.73 (m, 1H), 4.71 − 4.67 (m, 2H), 4.48 (d = 8.0 Hz, 2H), 4.41 (t, 2H), 4.01 (s, 3H), 3.26 (s, 2H). (ESI) m/z. [M + H]+ 490.1 |
| | PA153 | |
| PA154 | 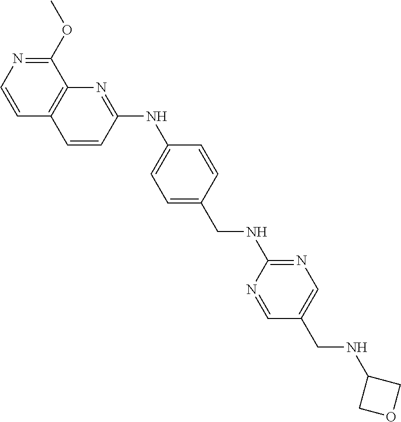 [SMILES: COc1nccc2ccc(Nc3ccc(CNc4ncc(CNC5COC5)cn4)cc3)nc12] | ¹H NMR (400 MHz, DMSO-d6) δ (ppm) 9.62 (s, 1H), 8.32 (s, 1H), 8.02 (d, 1H), 7.97 (d, 2H), 7.84 (d, 1H), 7.31 (d, 2H), 7.26 − 7.20 (m, 2H), 7.07 (d, 2H), 6.57 (d, 2H), 6.21 (br s, 1H), 4.22 (br s, 2H), 4.03 (s, 3H), 3.81 (br s, 2H), 3.69 (s, 2H), 3.29 − 3.22 (m, 2H), 2.84 − 2.75 (m, 1H), 1.82 (br d, 2H), 1.43 − 1.30 (m, 2H) (ESI) m/z. [M + H]+ 444.1 |
| | PA154 | |
| PA155 | 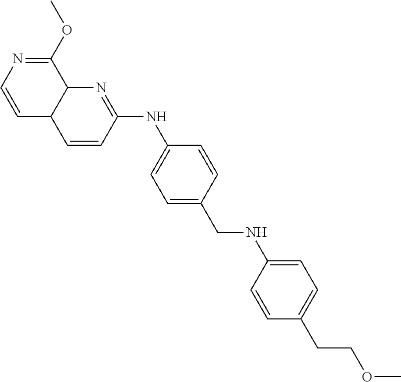 [SMILES: COCCc1ccc(NCc2ccc(NC3=NC4C(OC)=NC=CC4C=C3)cc2)cc1] | ¹H NMR (400 MHz, DMSO-d6) δ (ppm) 9.59 (s, 1H), 8.01 (d, 1H), 7.98 (s, 1H), 7.95 (s, 1H), 7.84 (d, 1H), 7.31 (d, 2H), 7.26 − 7.19 (m, 2H), 6.89 (d, 2H), 6.51 (d, 2H), 6.00 (br t, 1H), 4.19 (br d, 2H), 4.03 (s, 3H), 3.44 − 3.40 (m, 2H), 3.20 (s, 3H), 2.63 − 2.58 (m, 2H) (ESI) m/z. [M + H]+ 415.1 |
| | PA155 | |
| PA156 | 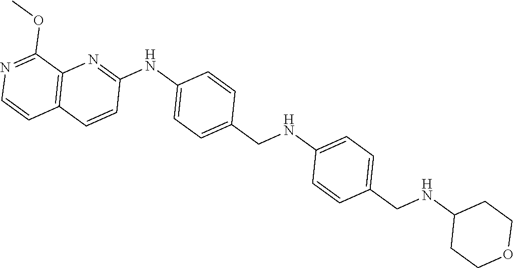 [SMILES: COc1nccc2ccc(Nc3ccc(CNc4ccc(CNC5CCOCC5)cc4)cc3)nc12] | ¹H NMR (400 MHz, DMSO-d6) δ (ppm) 9.62 (s, 1H), 8.32 (s, 1H), 8.02 (d, 1H), 7.97 (d, 2H), 7.84 (d, 1H), 7.31 (d, 2H), 7.26 − 7.20 (m, 2H), 7.07 (d, 2H), 6.57 (d, 2H), 6.21 (br s, 1H), 4.22 (br s, 2H), 4.03 (s, 3H), 3.81 (br s, 2H), 3.69 (s, 2H), 3.29 − 3.22 (m, 2H), 2.84 − 2.75 (m, 1H), 1.82 (br d, 2H), 1.43 − 1.30 (m, 2H) (ESI) m/z. [M + H]+ 470.2 |
| | PA156 | |
| PA157 | 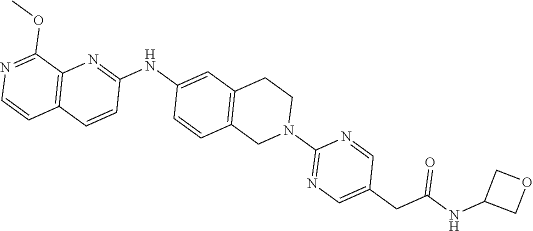 [SMILES: COc1nccc2ccc(Nc3ccc4c(c3)CCN(c3ncc(CC(=O)NC5COC5)cn3)C4)nc12] | ¹H NMR (400 MHz, DMSO-d6) δ (ppm) 9.54 (s, 1H), 8.77 (d, 1H), 8.24 (s, 2H), 8.07 − 7.88 (m, 2H), 7.81 (d, 1H), 7.74 (d, 1H), 7.17 (m, 3H), 4.81 − 4.63 (m, 5H), 4.36 (s, 2H), 4.01 (s, 3H), 3.96 (t, 2H), 3.27 (s, 2H), 2.85 (t, 2H). (ESI) m/z. [M + H]+ 498.1 |
| | PA157 | |
| PA158 | 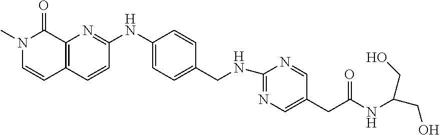 [SMILES: Cn1ccc2ccc(Nc3ccc(CNc4ncc(CC(=O)NC(CO)CO)cn4)cc3)nc2c1=O] | ¹H NMR (400 MHz, DMSO-d6) δ (ppm) 8.20 (s, 2H), 7.90 (d, 1H), 7.83 (d, 2H), 7.35 (d, 1H), 7.26 (d, 2H), 7.16 (d, 1H), 6.51 (d, 1H), 4.45 (s, 2H), 4.23 − 4.11 (m, 1H), 3.64 − 3.46 (m, 8H), 3.36 (s, 1H). (ESI) m/z. [M + H]+ 490.2 |
| | PA158 | |
| PA159 | 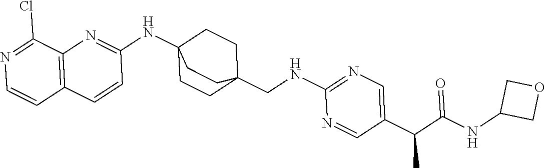 [SMILES: C[C@H](C(=O)NC1COC1)c1cnc(NCC23CCC(Nc4ccc5ccnc(Cl)c5n4)(CC2)CC3)nc1] | ¹H NMR (400 MHz, DMSO-d6) δ (ppm) 8.73 (d, 1H), 8.15 (s, 2H), 7.95 (d, 1H), 7.86 (d, 1H), 7.53 (d, 1H), 7.16 (s, 1H), 7.01 (d, 1H), 6.97 (t, 1H), 4.68-4.79 (m, 2H), 4.62-4.68 (m, 1H), 4.41 (t, 1H), 4.34 (t, 1H), 3.41 (q, 1H), 3.12 (d, 2H), 2.04-2.14 (m, 6H), 1.49-1.59 (m, 6H), 1.29 (d, 3H) (ESI) m/z. [M + H]+ 522.1 |
| | PA159 | |
| PA160 | 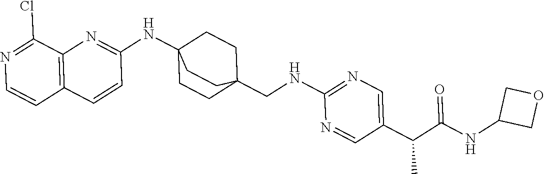 [SMILES: C[C@@H](C(=O)NC1COC1)c1cnc(NCC23CCC(Nc4ccc5ccnc(Cl)c5n4)(CC2)CC3)nc1] | ¹H NMR (400 MHz, DMSO-d6) δ (ppm) 8.73 (d, 1H), 8.15 (s, 2H), 7.95 (d, 1H), 7.86 (d, 1H), 7.53 (d, 1H), 7.16 (s, 1H), 7.01 (d, 1H), 6.97 (t, 1H), 4.68-4.79 (m, 2H), 4.62-4.68 (m, 1H), 4.41 (t, 1H), 4.34 (t, 1H), 3.41 (q, 1H), 3.12 (d, 2H), 2.04-2.14 (m, 6H), 1.49-1.59 (m, 6H), 1.29 (d, 3H) (ESI) m/z. [M + H]+ 522.1 |
| | PA160 | |
| PA161 | 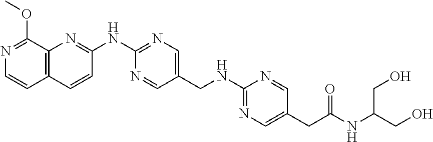 [SMILES: COc1nccc2ccc(Nc3ncc(CNc4ncc(CC(=O)NC(CO)CO)cn4)cn3)nc12] | ¹H NMR (400 MHz, Deuterium Oxide) δ (ppm) 8.78 (s, 4H), 8.49 (d, 1H), 8.36 (s, 3H), 8.04 (d, 1H), 7.61 (d, 1H), 7.39 (d, 1H),4.32 (dd, 1H), 4.22 (dd, 1H), 4.13 (s, 6H), 3.74 (dd, 1H), 3.70 − 3.62 (m, 4H), 3.57 (s, 1H). (ESI) m/z. [M + H]+ 492.2 |
| | PA161 | |
| PA162 | 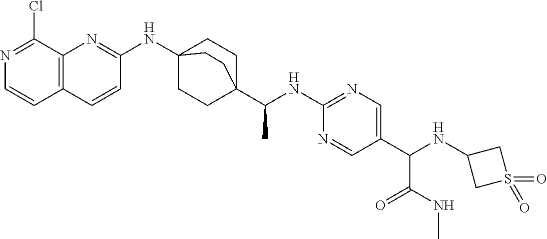 [SMILES: CNC(=O)C(NC1CS(=O)(=O)C1)c1cnc(N[C@@H](C)C23CCC(Nc4ccc5ccnc(Cl)c5n4)(CC2)CC3)nc1] | ¹H NMR (400 MHz, DMSO-d6) δ (ppm) 8.18 (s, 2H), 8.06 (dd, 1H), 7.95 (d, 1H), 7.86 (d, 1H), 7.53 (d, 1H), 7.18 (s, 1H), 7.01 (d, 1H), 6.88 (d, 1H), 4.30-4.19 (m, 2H), 4.00-3.90 (m, 4H), 2.60 (d, 3H), 2.08 (t, 6H), 1.65-1.51 (m, 6H), 1.01 (d, 3H); (ESI) m/z. [M + H]+ 599.4 |
| | PA162 | |
| PA163 | 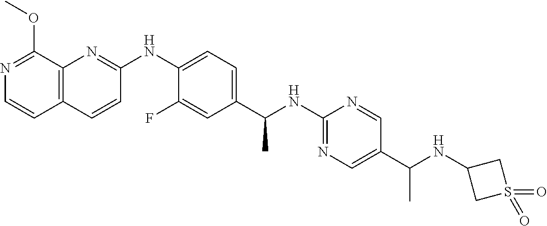 [SMILES: COc1nccc2ccc(Nc3ccc([C@H](C)Nc4ncc(C(C)NC5CS(=O)(=O)C5)cn4)cc3F)nc12] | ¹H NMR (400 MHz, DMSO-d6) δ (ppm) 9.19 (s, 1H), 8.63-8.58 (m, 1H), 8.18 (s, 2H), 8.01 (d, 1H), 7.82 (d, 1H), 7.62 (s, 1H), 7.39 (d, 1H), 7.27-7.14 (m, 3H), 5.08-5.01 (m, 1H), 4.33-4.08 (m, 2H), 3.97 (s, 3H), 3.94-3.66 (m, 2H), 3.47-3.43 (m, 1H), 3.28-3.30 (m, 1H) 1.41 (d, 3H), 1.21 (d, 3H); (ESI) m/z. [M + H]+ 538.3 |
| | PA163 | |
| PA164 | 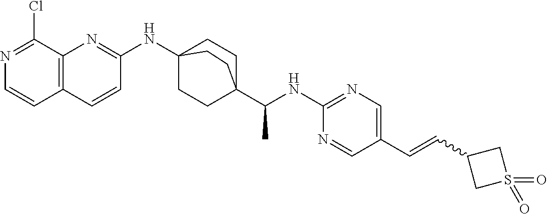 [SMILES: C[C@H](Nc1ncc(C=CC2CS(=O)(=O)C2)cn1)C12CCC(Nc3ccc4ccnc(Cl)c4n3)(CC1)CC2] | ¹H NMR (400 MHz, DMSO-d6) δ (ppm) 8.22 (s, 2H), 7.96 (s, 1H), 7.87 (d, 1H), 7.54 (s, 1H), 7.19(s, 1H), 7.10 (d, 1H), 7.03 (s, 1H), 6.30 (d, 1H), 5.83-5.74 (m, 1H), 4.55-4.46(m, 2H), 4.14-3.96 (m, 3H), 3.50 (d, 1H), 2.08 (s, 6H), 1.57 (dd, 6H), 1.03 (s, 3H); (ESI) m/z. [M + H]+ 539.3 |
| | PA164 | |
| PA165 | 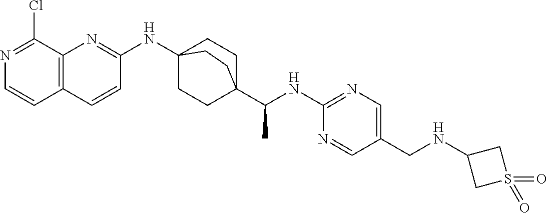 [SMILES: C[C@H](Nc1ncc(CNC2CS(=O)(=O)C2)cn1)C12CCC(Nc3ccc4ccnc(Cl)c4n3)(CC1)CC2] | ¹H NMR (400 MHz, DMSO-d6) δ (ppm) 8.17 (s, 2H), 7.95 (d, 1H), 7.86 (d, 1H), 7.53 (d, 1H), 7.18 (s, 1H), 7.02 (d, 1H), 6.78-6.71 (m, 1H), 4.30 (dd, 2H), 4.01-3.94 (m, 1H), 3.91 (dd, 2H), 3.54-3.45 (m, 1H), 3.40 (s, 2H), 2.08 (t, 6H), 1.67-1.49 (m, 6H), 1.01 (d, 3H); (ESI) m/z. [M + H]+ 542.3 |
| | PA165 | |
| PA166 | 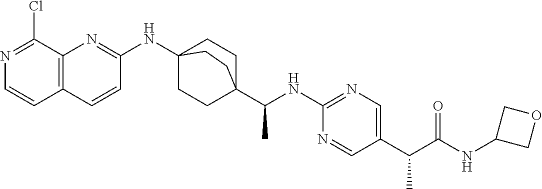 [SMILES: C[C@H](Nc1ncc([C@@H](C)C(=O)NC2COC2)cn1)C12CCC(Nc3ccc4ccnc(Cl)c4n3)(CC1)CC2] | ¹H NMR (400 MHz, DMSO-d6) δ (ppm) 8.75 (d, 1H), 8.15 (s, 2H), 7.96 (d, 1H), 7.87 (d, 1H), 7.53 (d, 1H), 7.18 (s, 1H), 7.02 (d, 1H), 6.74 (d, 1H), 4.79-4.64 (m, 3H), 4.41 (t, 1H), 4.36 (t, 1H), 3.98-3.89 (m, 1H), 3.42 (t, 1H), 2.11-2.05 (m, 6H), 1.62-1.51 (m, 6H), 1.30 (d, 3H), 1.01 (d, 3H); (ESI) m/z. [M + H]+ 536.0 |
| | PA166 | |
| PA167 | 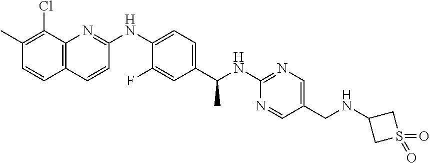 [SMILES: Cc1ccc2ccc(Nc3ccc([C@H](C)Nc4ncc(CNC5CS(=O)(=O)C5)cn4)cc3F)nc2c1Cl] | ¹H NMR (400 MHz, DMSO-d6) δ (ppm) 9.20 (d, 1H), 9.08 (t, 1H), 8.16 (s, 2H), 8.04 (d, 1H), 7.59 (dd, 2H), 7.30 (d, 1H), 7.27-7.17 (m, 3H), 5.05 (p, 1H), 4.28-4.20 (m, 2H), 3.84 (ddd, − 2H), 3.46-3.40 (m, 1H), 3.36 (s, 2H), 2.49 (s, 3H), 1.41 (d, 3H); (ESI) m/z. [M + H]+ 541.2 |
| | PA167 | |
| PA168 | 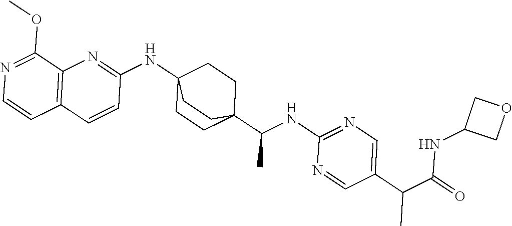 [SMILES: COc1nccc2ccc(NC34CCC([C@H](C)Nc5ncc(C(C)C(=O)NC6COC6)cn5)(CC3)CC4)nc12] | ¹H NMR (400 MHz, DMSO-d6) δ (ppm) 8.71 (d, 1H), 8.11 (s, 2H), 7.69 (d, 1H), 7.64 (d, 1H), 7.05-7.00 (m, 1H), 6.88 (d, 1H), 6.74-6.64 (m, 2H), 4.74-4.60 (m, 3H), 4.34 (dt, 2H), 3.92 (s, 3H), 3.89 (dd, 1H), 3.41-3.34 (m, 1H), 2.00-1.95 (m, 6H), 1.56-1.45 (m, 6H), 1.26 (d, 3H), 0.97 (d, 3H); (ESI) m/z. [M + H]+ 532.1 |
| | PA168 | |
| PA169 | 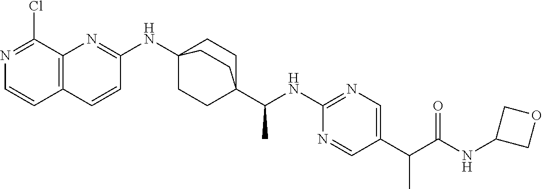 [SMILES: CC(C(=O)NC1COC1)c1cnc(N[C@@H](C)C23CCC(Nc4ccc5ccnc(Cl)c5n4)(CC2)CC3)nc1] | ¹H NMR (400 MHz, DMSO-d6) δ (ppm) 8.75 (d, 1H), 8.15 (s, 2H), 7.96 (d, 1H), 7.87 (d, 1H), 7.53 (d, 1H), 7.18 (s, 1H), 7.02 (d, 1H), 6.74 (d, 1H), 4.79-4.64 (m, 3H), 4.41 (t, 1H), 4.36 (t, 1H), 3.98-3.89 (m, 1H), 3.42 (t, 1H), 2.11-2.05 (m, 6H), 1.62-1.51 (m, 6H), 1.30 (d, 3H), 1.01 (d, 3H); (ESI) m/z. [M + H]+ 536.0 |
| | PA169 | |
| PA170 | 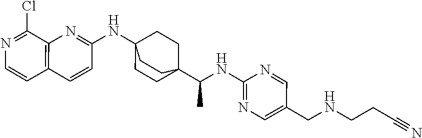 [SMILES: C[C@H](Nc1ncc(CNCCC#N)cn1)C12CCC(Nc3ccc4ccnc(Cl)c4n3)(CC1)CC2] | ¹H NMR (400 MHz, DMSO-d6) δ (ppm) 8.18 (s, 2H), 7.96 (d, 1H), 7.87 (d, 1H), 7.53 (d, 1H), 7.19 (s, 1H), 7.02 (d, 1H), 6.70 (d, 1H), 3.97 (s, 1H), 3.49 (s, 2H), 2.70 (t, 2H), 2.59 (t, 2H), 2.08 (t, 6H), 1.58 (d, 6H), 1.02 (d, 3H); (ESI) m/z. [M + H]+ 491.2 |
| | PA170 | |
| PA171 | 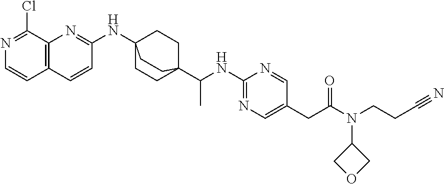 [SMILES: CC(Nc1ncc(CC(=O)N(CCC#N)C2COC2)cn1)C12CCC(Nc3ccc4ccnc(Cl)c4n3)(CC1)CC2] | ¹H NMR (400 MHz, DMSO-d6) δ (ppm) 8.05 (d, 2H), 7.96 (d, 1H), 7.86 (d, 1H), 7.53 (d, 1H), 7.17 (s, 1H), 7.02 (d, 1H), 6.68 (d, 1H), 5.07 (d, 1H), 4.66 (d, 4H), 3.96 (s, 1H), 3.78 (s, 2H), 3.54 (d, 2H), 2.82 (s, 2H), 2.08 (s, 6H), 1.58 (d, 6H), 1.02 (d, 3H); (ESI) m/z. [M + H]+ 575.4 |
| | PA171 | |
| PA172 | 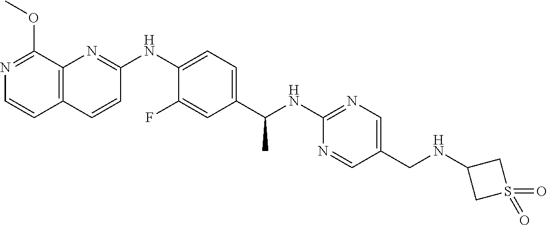 [SMILES: COc1nccc2ccc(Nc3ccc([C@H](C)Nc4ncc(CNC5CS(=O)(=O)C5)cn4)cc3F)nc12] | ¹H NMR (400 MHz, DMSO-d6) δ (ppm) 9.23 (s, 1H), 8.64 (t, 1H), 8.20 (s, 2H), 8.05 (d, 1H), 7.85 (d, 1H), 7.64 (d, 1H), 7.43 (d, 1H), 7.31-7.17 (m, 3H), 5.08 (p, 1H), 4.28 (dd, 2H), 4.01 (s, 3H), 3.88 (dd, 2H), 3.46 (ddd, 1H), 3.39 (s, 2H), 1.45 (d, 3H). (ESI) m/z. [M + H]+ 524.3 |
| | PA172 | |
| PA173 | 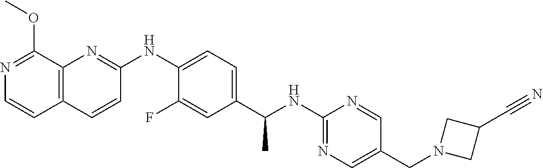 [SMILES: COc1nccc2ccc(Nc3ccc([C@H](C)Nc4ncc(CN5CC(C#N)C5)cn4)cc3F)nc12] | ¹H NMR (400 MHz, DMSO-d6) δ (ppm) 9.22 (s, 1H), 8.65 (t, 1H), 8.15 (s, 2H), 8.05 (d, 1H), 7.85 (d, 1H), 7.67 (d, 1H), 7.44 (d, 1H), 7.30-7.18 (m, 3H), 5.12-5.08 (m, 1H), 4.01 (s, 3H), 3.45-3.43 (m, 1H), 3.41-3.38 (m, 2H), 3.37-3.36 (m, 2H), 3.23-3.20 (m, 2H), 1.44 (d, 3H). (ESI) m/z. [M + H]+ 485.0 |
| | PA173 | |
| PA174 | 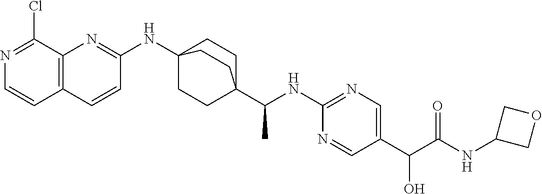 [SMILES: C[C@H](Nc1ncc(C(O)C(=O)NC2COC2)cn1)C12CCC(Nc3ccc4ccnc(Cl)c4n3)(CC1)CC2] | ¹H NMR (400 MHz, DMSO-d6) δ (ppm) 8.74 (d, 1H), 8.16 (s, 2H), 7.92 (d, 1H), 7.83 (d, 1H), 7.49 (d, 1H), 7.14 (s, 1H), 6.98 (d, 1H), 6.83 (d, 1H), 4.83-4.72 (m, 2H), 4.66-4.59 (m, 2H), 4.49 (t, 2H), 3.93 (dd, 1H), 2.04 (t, 6H), 1.99-1.94 (m, 1H), 1.58-1.47 (m, 6H), 0.97 (d, 3H); (ESI) m/z. [M + H]+ 539.0 |
| | PA174 | |
| PA175 | 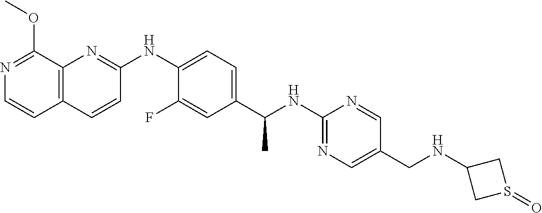 [SMILES: COc1nccc2ccc(Nc3ccc([C@H](C)Nc4ncc(CNC5CS(=O)C5)cn4)cc3F)nc12] | ¹H NMR (400 MHz, DMSO-d6) δ (ppm) 9.22 (s, 1H), 8.69-8.60 (m, 1H), 8.22 (d, 2H), 8.05 (d, 1H), 7.86 (d, 1H), 7.71 (d, 1H), 7.43 (d, 1H), 7.27-7.19 (m, 3H), 5.14-5.04 (m, 1H), 4.01 (s, 3H), 4.00-3.90 (m, 2H), 3.59-3.42 (m, 2H), 3.22-3.14 (m, 1H), 3.12-2.94 (m, 2H), 1.45 (d, 3H); (ESI) m/z. [M + H]+ 508.0 |
| | PA175 | |
| PA176 | 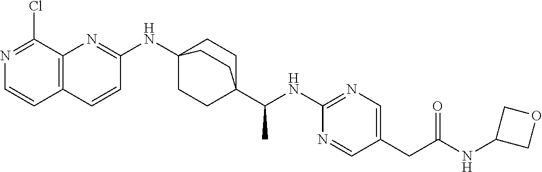 [SMILES: C[C@H](Nc1ncc(CC(=O)NC2COC2)cn1)C12CCC(Nc3ccc4ccnc(Cl)c4n3)(CC1)CC2] | ¹H NMR (400 MHz, DMSO-d6) δ (ppm) 8.80 (d, 1H), 8.09 (d, 2H), 7.95 (d, 1H), 7.86 (d, 1H), 7.53 (d, 1H), 7.18 (s, 1H), 7.02 (d, 1H), 6.70 (d, 1H), 4.82-4.72 (m, 1H), 4.70 (t, 2H), 4.41 (t, 2H), 3.98-3.89 (m, 1H), 3.23 (s, 2H), 2.11-2.05 (m, 6H), 1.62-1.52 (m, 6H), 1.01 (d, 3H). (ESI) m/z. [M + H]+ 522.20 |
| | PA176 | |
| PA177 | 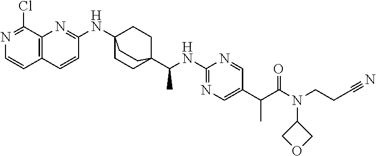 [SMILES: CC(C(=O)N(CCC#N)C1COC1)c1cnc(N[C@@H](C)C23CCC(Nc4ccc5ccnc(Cl)c5n4)(CC2)CC3)nc1] | ¹H NMR (400 MHz, DMSO-d6) δ (ppm) 8.15 (s, 1H), 8.08 (s, 1H), 7.92 (d, 1H), 7.83 (d, 1H), 7.49 (d, 1H), 7.14 (s, 1H), 6.98 (d, 1H), 6.75 (d, 1H), 4.80-4.21 (m, 5H), 4.01-3.68 (m, 4H), 3.59 (d, 1H), 2.80-2.74 (m, 1H), 2.09-1.99 (m, 6H), 1.54 (d, 6H), 1.26-1.18 (m, 3H), 0.97 (d, 3H). (ESI) m/z. [M + H]+ 589.2 |
| | PA177 | |
| PA179 | 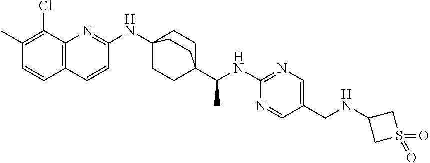 [SMILES: Cc1ccc2ccc(NC34CCC([C@H](C)Nc5ncc(CNC6CS(=O)(=O)C6)cn5)(CC3)CC4)nc2c1Cl] | ¹H NMR (400 MHz, DMSO-d6) δ (ppm) 8.15 (s, 2H), 7.71 (d, 1H), 7.39 (d, 1H), 7.03 (d, 1H), 6.76 (s, 1H), 6.73-6.67 (m, 2H), 4.40-4.20 (m, 2H), 3.93 (dq, 3H), 3.48 (d, 3H), 2.42 (s, 3H), 2.05 (t, 6H), 1.64-1.43 (m, 6H), 0.98 (d, 3H). (ESI) m/z. [M + H]+ 555.4 |
| | PA179 | |
| [symbol] | 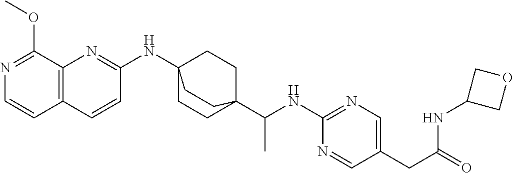 [SMILES: COc1nccc2ccc(NC34CCC(C(C)Nc5ncc(CC(=O)NC6COC6)cn5)(CC3)CC4)nc12] | [symbol] |
| | PA180 | |
| PA181 | 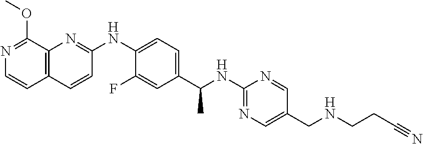 [SMILES: COc1nccc2ccc(Nc3ccc([C@H](C)Nc4ncc(CNCCC#N)cn4)cc3F)nc12] | ¹H NMR (400 MHz, DMSO-d6) δ (ppm) 9.21 (s, 1H), 8.64 (t, 1H), 8.21 (s, 2H), 8.04 (d, 1H), 7.85 (d, 1H), 7.58 (d, 1H), 7.43 (d, 1H), 7.33-7.18 (m, 3H), 5.09 (p, 1H), 4.01 (s, 3H), 3.49 (s, 2H), 2.67 (t, 2H), 2.57 (t, 2H), 1.45 (d, 3H); (ESI) m/z. [M + H]+ 473.1 |
| | PA181 | |
| PA182 | 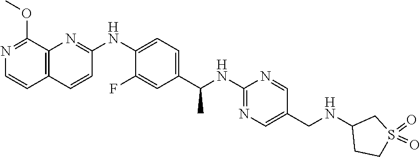 [SMILES: COc1nccc2ccc(Nc3ccc([C@H](C)Nc4ncc(CNC5CCS(=O)(=O)C5)cn4)cc3F)nc12] PA182 | ¹H NMR (400 MHz, DMSO-d6) δ (ppm) 9.23 (d, 1H), 8.64 (t, 1H), 8.23 (s, 2H), 8.05 (d, 1H), 7.85 (d, 1H), 7.68 (s, 1H), 7.43 (d, 1H), 7.29-7.19 (m, 3H), 5.07 (q, 1H), 4.01 (s, 3H), 3.61-3.46 (m, 2H), 3.44-3.35 (m, 2H), 3.27-3.19 (m, 1H), 3.11-3.01 (m, 1H), 2.98-2.82 (m, 1H), 2.35-2.21 (m, 1H), 2.05-1.90 (m, 1H), 1.45 (d, 3H); (ESI) m/z. [M + H]+ 538.0 |
| PA183 | 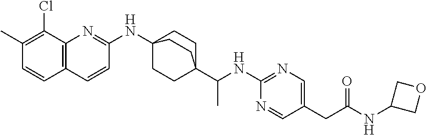 [SMILES: Cc1ccc2ccc(NC34CCC(C(C)Nc5ncc(CC(=O)NC6COC6)cn5)(CC3)CC4)nc2c1Cl] | ¹H NMR (400 MHz, DMSO-d6) δ (ppm) 8.79 (d, 1H), 8.09 (s, 2H), 7.74 (d, 1H), 7.42 (d, 1H), 7.05 (d, 1H), 6.70 (dd, 3H), 4.76 (q, 1H), 4.69 (t, 2H), 4.40 (d, 2H), 3.98-3.88 (m, 1H), 3.22 (s, 2H), 2.45 (s, 3H), 2.08 (t, 6H), 1.60-1.51 (m, 6H), 1.00 (d, 3H). (ESI) m/z. [M + H]⁺ 535.2 |
| | PA183 | |
| PA184 | 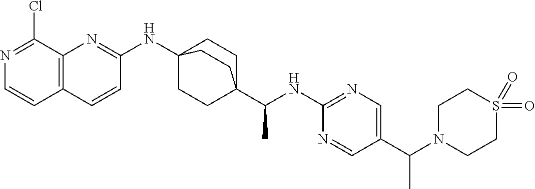 [SMILES: CC(c1cnc(N[C@@H](C)C23CCC(Nc4ccc5ccnc(Cl)c5n4)(CC2)CC3)nc1)N1CCS(=O)(=O)CC1] | ¹H NMR (400 MHz, DMSO-d6) δ (ppm) 8.18 (s, 2H), 7.92 (d, 1H), 7.83 (d, 1H), 7.50 (d, 1H), 7.15 (s, 1H), 6.98 (d, 1H), 6.79-6.73 (m, 1H), 3.93 (dd, 1H), 3.80-3.72 (m, 1H), 3.03 (t, 4H), 2.94-2.67 (m, 4H), 2.04 (t, 6H), 1.62-1.45 (m, 6H), 1.27 (d, 3H), 0.98 (d, 3H). (ESI) m/z. [M + H]⁺ 570.3 |
| | PA184 | |
| PA185 | 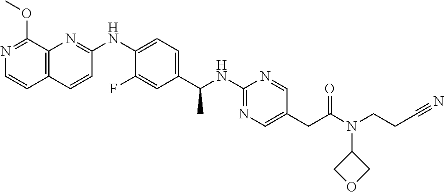 [SMILES: COc1nccc2ccc(Nc3ccc([C@H](C)Nc4ncc(CC(=O)N(CCC#N)C5COC5)cn4)cc3F)nc12] | ¹H NMR (400 MHz, DMSO-d6) δ (ppm) 9.20 (s, 1H), 8.64 (t, 1H), 8.06 (dd, 3H), 7.85 (d, 1H), 7.55 (d, 1H), 7.44 (dd, 1H), 7.30-7.16 (m, 3H), 5.23-5.44 (m, 0.4H), 5.08-5.20 (m, 1H), 4.97-5.17 (m, 1H), 4.76-4.85 (m, 0.4H), 4.69 (d, 2H), 4.59 (d, 2H), 4.01 (s, 3H), 3.75 (dt, 2H), 3.54 (d, 2H), 2.79 (dt, 2H), 1.45 (d, 3H). (ESI) m/z. [M + H]⁺ 557.2 |
| | PA185 | |
| PA186 | 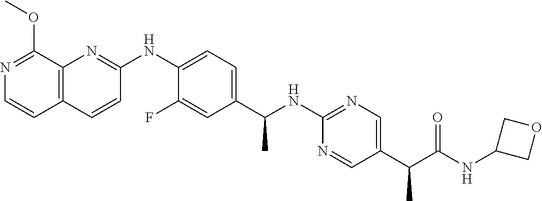 [SMILES: COc1nccc2ccc(Nc3ccc([C@H](C)Nc4ncc([C@H](C)C(=O)NC5COC5)cn4)cc3F)nc12] | ¹H NMR (400 MHz, DMSO-d6) δ (ppm) 9.16 (s, 1H), 8.69-8.57 (m, 2H), 8.14 (s, 2H), 8.01 (d, 1H), 7.82 (d, 1H),7.56 (d, 1H), 7.40 (d, 1H), 7.25-7.15 (m, 3H), 5.08-4.99 (m, 1H), 4.71-4.62 (m, 2H),4.59 (d, 1H), 4.32 (dt, 2H), 3.97 (s, 3H), 3.37 (d, 1H), 1.41 (d, 3H), 1.24 (d, 3H); (ESI) m/z. [M + H]+ 518.2 |
| | PA186 | |
| PA187 | 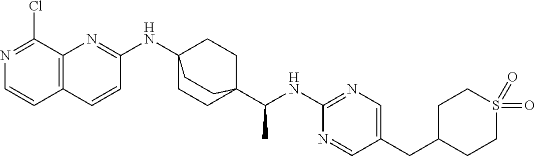 [SMILES: C[C@H](Nc1ncc(CC2CCS(=O)(=O)CC2)cn1)C12CCC(Nc3ccc4ccnc(Cl)c4n3)(CC1)CC2] | ¹H NMR (400 MHz, DMSO-d6) δ (ppm) 8.05 (s, 2H), 7.92 (d, 1H), 7.83 (d, 1H), 7.50 (d, 1H), 7.14 (s, 1H), 6.98 (d, 1H), 6.60 (d, 1H), 3.94-3.87 (m, 1H), 3.07-2.95 (m, 4H), 2.32-2.28 (m, 2H),2.04 (t, 6H), 1.89 (d, 2H), 1.76-1.66 (m, 1H), 1.53 (dt, 8H), 0.97 (d, 3H). (ESI) m/z. [M + H]⁺ 555.0 |
| | PA187 | |
| PA188 | 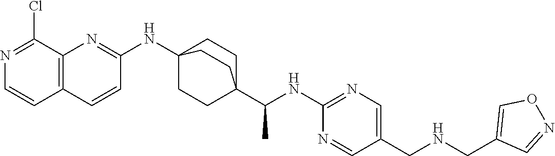 [SMILES: C[C@H](Nc1ncc(CNCc2cnoc2)cn1)C12CCC(Nc3ccc4ccnc(Cl)c4n3)(CC1)CC2] | ¹H NMR (400 MHz, Methanol-d4) δ (ppm) 8.72 (s, 1H), 8.46 (s, 1H), 8.27 (s, 2H), 7.87 (d, 1H), 7.74 (d, 1H), 7.44 (d, 1H), 6.90 (d, 1H), 3.96 (t, 1H), 3.90 (s, 2H), 3.79 (s, 2H), 2.21-2.14 (m, 6H), 1.69-1.62 (m, 6H), 1.09 (d, 3H). (ESI) m/z. [M + H]+ 519.2 |
| | PA188 | |
| PA189 | 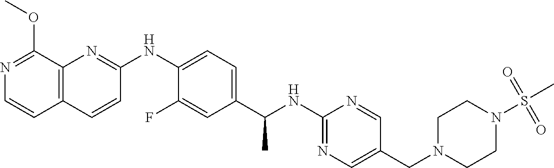 [SMILES: COc1nccc2ccc(Nc3ccc([C@H](C)Nc4ncc(CN5CCN(S(C)(=O)=O)CC5)cn4)cc3F)nc12] | ¹H NMR (400 MHz, DMSO-d6) δ (ppm) 9.20 (s, 1H), 8.63 (t, 1H), 8.14 (s, 2H), 8.01 (d, 1H), 7.82 (d, 1H), 7.66 (d, 1H), 7.41 (d, 1H), 7.27-7.14 (m, 3H), 5.14-4.98 (m, 1H), 3.97 (s, 3H), 3.28 (s, 2H), 3.07-2.99 (m, 4H), 2.80 (s, 3H), 2.43-2.30 (m, 4H), 1.41 (d, 3H). (ESI) m/z. [M + H]⁺ 567.4 |
| | PA189 | |
| PA190 | 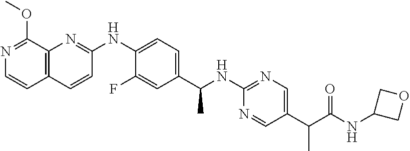 [SMILES: COc1nccc2ccc(Nc3ccc([C@H](C)Nc4ncc(C(C)C(=O)NC5COC5)cn4)cc3F)nc12] | ¹H NMR (400 MHz, DMSO-d6) δ (ppm) 9.16 (s, 1H), 8.75-8.49 (m, 2H), 8.14 (d, 2H), 8.00 (d, 1H), 7.87-7.77 (m, 1H), 7.56 (d, 1H), 7.39 (d, 1H), 7.24-7.16 (m, 3H), 5.07-4.99 (m, 1H), 4.66 (dt, 2H),4.60 (t, 1H), 4.39-4.25 (m, 2H), 3.97 (s, 3H), 3.37 (d, 1H), 1.41 (d, 3H), 1.24 (d, 3H). (ESI) m/z. [M + H]⁺ 518.0 |
| | PA190 | |
| PA191 | 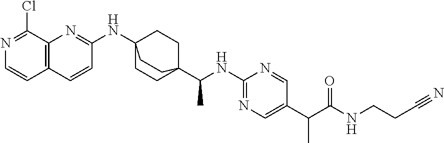 [SMILES: CC(C(=O)NCCC#N)c1cnc(N[C@@H](C)C23CCC(Nc4ccc5ccnc(Cl)c5n4)(CC2)CC3)nc1] | ¹H NMR (400 MHz, DMSO-d6) δ (ppm) 8.36 (t, 1H), 8.15 (s, 2H), 7.95 (d, 1H), 7.86 (d, 1H), 7.53 (d, 1H), 7.18 (s, 1H), 7.02 (d, 1H), 6.74 (d, 1H), 3.98-3.90 (m, 1H), 3.42 (q, 1H), 3.27-3.21 (m, 2H), 2.63 (t, 2H), 2.07 (t, 6H), 1.65-1.50 (m, 6H), 1.31 (d, 3H), 1.00 (d, 3H); (ESI) m/z. [M + H]+ 533.2 |
| | PA191 | |
| PA192 | 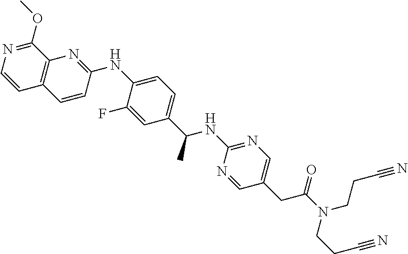 [SMILES: COc1nccc2ccc(Nc3ccc([C@H](C)Nc4ncc(CC(=O)N(CCC#N)CCC#N)cn4)cc3F)nc12] | ¹H NMR (400 MHz, DMSO-d6) δ (ppm) 9.21 (s, 1H), 8.64 (t, 1H), 8.11-7.99 (m, 3H), 7.84 (d, 1H), 7.57 (d, 1H), 7.44 (dd, 1H), 7.31-7.17 (m, 3H), 5.15-5.01 (m, 1H), 4.00 (s, 3H), 3.75 (t, 2H), 3.60 (s, 2H), 3.54 (t, 2H), 2.89 (t, 2H), 2.70 (t, 2H), 1.44 (d, 3H). (ESI) m/z. [M + H]+ 554.2 |
| | PA192 | |
| PA193 | 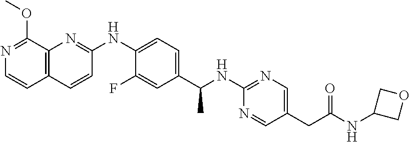 [SMILES: COc1nccc2ccc(Nc3ccc([C@H](C)Nc4ncc(CC(=O)NC5COC5)cn4)cc3F)nc12] | ¹H NMR (400 MHz, DMSO-d6) δ (ppm) 9.20 (s, 1H), 8.76 (d, 1H), 8.64 (t, 1H), 8.12 (s, 2H), 8.04 (d, 1H), 7.85 (d, 1H), 7.57 (d, 1H), 7.43 (d, 1H), 7.30-7.14 (m, 3H), 5.07 (t, 1H), 4.79-4.70 (m, 1H), 4.67 (t, 2H), 4.39 (t, 2H), 4.01 (s, 3H), 3.23 (s, 2H), 1.44 (d, 3H); (ESI) m/z. [M + H]+ 504.0 |
| | PA193 | |
| PA194 | 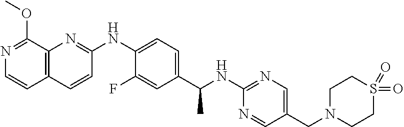 [SMILES: COc1nccc2ccc(Nc3ccc([C@H](C)Nc4ncc(CN5CCS(=O)(=O)CC5)cn4)cc3F)nc12] PA194 | ¹H NMR (400 MHz, DMSO-d6) δ (ppm) 9.18 (d, 0.4H), 8.63 (dt, 1H), 8.17 (s, 2H), 8.01 (dd, 1H), 7.82 (dd, 1H), 7.66 (d, 0.4H), 7.41 (dd, 1H), 7.26-7.15 (m, 3H), 5.05 (q, 1H), 3.97 (s, 3H), 3.42 (s, 2H), 3.10-3.00 (m, 4H), 2.88-2.74 (m, 4H), 1.41 (d, 3H). (ESI) m/z. [M + H]+ 538.0 |
| PA195 | 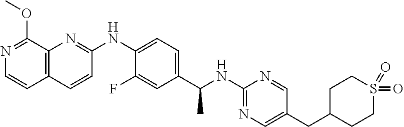 [SMILES: COc1nccc2ccc(Nc3ccc([C@H](C)Nc4ncc(CC5CCS(=O)(=O)CC5)cn4)cc3F)nc12] PA195 | ¹H NMR (400 MHz, DMSO-d6) δ (ppm) 9.17 (s, 1H), 8.61 (t, 1H), 8.08 (s, 2H), 8.01 (d, 1H),7.82 (d, 1H), 7.50 (d, 1H), 7.40 (dd, 1H), 7.25-7.14 (m, 3H), 5.09-4.97 (m, 1H), 3.97 (s, 3H), 3.04-2.89 (m, 4H), 2.31 (d, 2H), 1.91-1.81 (m, 2H), 1.76-1.65 (m, 1H), 1.63-1.47 (m, 2H), 1.41 (d, 3H); (ESI) m/z. [M + H]+ 537.1 |
| PA196 | 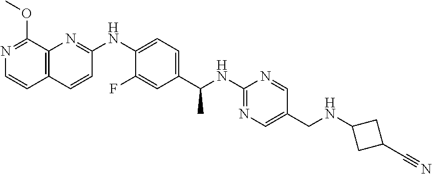 [SMILES: COc1nccc2ccc(Nc3ccc([C@H](C)Nc4ncc(CNC5CC(C#N)C5)cn4)cc3F)nc12] | ¹H NMR (400 MHz, DMSO-d6) δ (ppm) 9.87-9.78 (m, 1H), 9.15 (d, 1H),8.82 (d, 1H), 8.40 (dd, 1H), 8.15 (s, 2H), 8.07 (d, 1H), 7.87 (d, 1H), 7.56 (t, 1H), 7.32-7.22 (m, 3H), 4.80-4.72 (m, 1H), 4.71-4.63 (m, 2H), 4.54 (d, 2H), 4.40 (t, 2H), 4.04 (s, 3H), 3.26 (s, 2H); (ESI) m/z. [M + H]+ 499.3 |
| | PA196 | |
| PA197 | 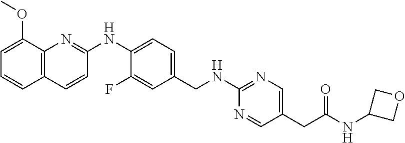 [SMILES: COc1cccc2ccc(Nc3ccc(CNc4ncc(CC(=O)NC5COC5)cn4)cc3F)nc12] | ¹H NMR (400 MHz, DMSO-d6) δ (ppm) 8.99 (s, 1H), 8.90-8.72 (m, 2H), 8.15 (s, 2H), 8.01 (d, 1H), 7.60 (s, 1H), 7.31-7.03 (m, 6H), 4.71 (dt, 3H), 4.48-4.36 (m, 4H), 3.91 (s, 3H), 3.25 (s, 2H). (ESI) m/z. [M + H]+ 489.1 |
| | PA197 | |
| PA198 | 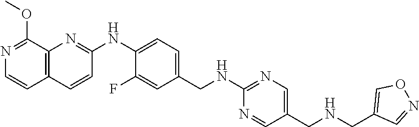 [SMILES: COc1nccc2ccc(Nc3ccc(CNc4ncc(CNCc5cnoc5)cn4)cc3F)nc12] PA198 | ¹H NMR (400 MHz, DMSO-d6) δ (ppm) 9.22 (s, 1H), 8.81 (s, 1H), 8.69-8.61 (m, 1H), 8.56 (s, 1H), 8.25 (s, 2H), 8.14 (s, 1H), 8.04 (d, 1H), 7.85 (d, 1H), 7.68 (s, 1H), 7.43 (d, 1H), 7.23 (d, 1H), 7.20-7.11 (m, 2H), 4.48 (d, 2H), 3.99 (s, 3H), 3.60 (s, 2H), 3.53 (s, 2H). (ESI) m/z. [M + H]⁺ 487.1 |
| PA199 | 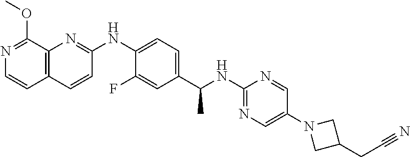 [SMILES: COc1nccc2ccc(Nc3ccc([C@H](C)Nc4ncc(N5CC(CC#N)C5)cn4)cc3F)nc12] | ¹H NMR (400 MHz, DMSO-d6) δ (ppm) 9.20 (s, 1H), 8.59 (d, 1H), 8.03 (d, 1H), 7.83 (d, 1H), 7.73 (s, 2H), 7.41 (d, 1H), 7.25-7.15 (m, 4H), 7.07(s, 1H), 6.94 (s, 1H), 4.99-4.90 (m, 1H), 3.99 (s, 3H), 3.87 (d, 2H), 3.48-3.43 (m, 2H), 3.00-2.89 (m, 1H), 2.84 (d, 2H), 1.41 (d, 3H). (ESI) m/z. [M + H]+ 485.3 |
| | PA199 | |
| PA200 | 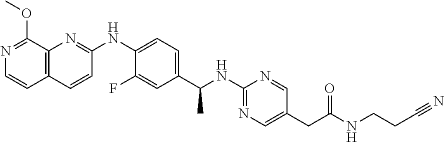 [SMILES: COc1nccc2ccc(Nc3ccc([C@H](C)Nc4ncc(CC(=O)NCCC#N)cn4)cc3F)nc12] | ¹H NMR (400 MHz, DMSO-d₆) δ (ppm) 9.21 (s, 0.3H), 8.72-8.58 (m, 1H), 8.42-8.33 (m, 1H), 8.13 (s, 2H), 8.04 (d, 1H), 7.85 (d, 1H), 7.58 (d, 1H), 7.44 (dd, 1H), 7.29-7.18 (m, 3H), 5.10-5.05 (m, 1H), 4.01 (s, 3H), 3.24 (s, 2H), 2.67 (ddd, 2H), 2.62 (t, 2H), 1.45 (d, 3H). (ESI) m/z. [M + H]+ 501.2 |
| | PA200 | |
| PA201 | 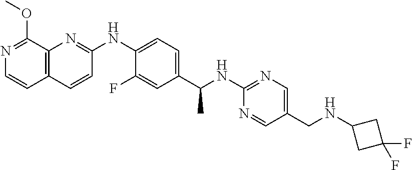 [SMILES: COc1nccc2ccc(Nc3ccc([C@H](C)Nc4ncc(CNC5CC(F)(F)C5)cn4)cc3F)nc12] | ¹H NMR (400 MHz, DMSO-d6) δ (ppm) 9.22 (s, 1H), 8.70-8.60 (m, 1H), 8.19 (s, 2H), 8.04 (d, 1H), 7.85 (d, 7.59 (d, 1H), 7.44 (dd, 1H), 7.30-7.19 (m, 3H), 5.17-5.01 (m, 1H), 4.01 (s, 3H), 3.38 (s, 2H), 3.13-2.98 (m, 1H), 2.81-2.61 (m, 2H), 2.40-2.17 (m, 2H), 1.50-1.40 (m, 3H). (ESI) m/z. [M + H]⁺ 510.0 |
| | PA201 | |
| PA202 | 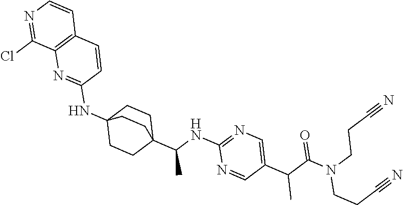 [SMILES: CC(C(=O)N(CCC#N)CCC#N)c1cnc(N[C@@H](C)C23CCC(Nc4ccc5ccnc(Cl)c5n4)(CC2)CC3)nc1] | ¹H NMR (400 MHz, DMSO-d6) δ (ppm) 8.20 (s, 2H), 7.95 (d, 1H), 7.87 (d, 1H), 7.53 (d, 1H), 7.18 (s, 1H), 7.02 (d, 1H), 6.77 (d, 1H), 4.04-3.90 (m, 2H), 3.81-3.70 (m, 1H), 3.69-3.58 (m, 2H), 3.49-3.43 (m, 1H), 2.93-2.74 (m, 2H), 2.72 (t, 2H), 2.07 (t, 6H), 1.65-1.50 (m, 6H), 1.29 (d, 3H), 1.00 (d, 3H). (ESI) m/z. [M + H]⁺ 586.3 |
| | PA202 | |
| PA203 | 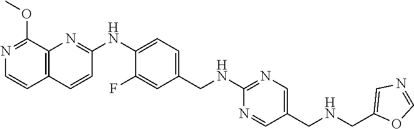 [SMILES: COc1nccc2ccc(Nc3ccc(CNc4ncc(CNCc5cnco5)cn4)cc3F)nc12] | ¹H NMR (400 MHz, DMSO-d6) δ (ppm) 9.23 (s, 1H), 8.66 (t, 1H), 8.37 (d, 1H), 8.34-8.27 (m, 2H), 8.14 (s, 0.5H), 8.05 (d, 1H), 7.86 (d, 1H), 7.44 (d, 1H), 7.24 (d, 1H), 7.22-7.12 (m, 3H), 6.53 (s, 0.4H), 4.54-4.45 (m, 2H), 4.00 (s, 5H), 3.72 (d, 2H); (ESI) m/z. [M + H]+ 487.20 |
| | PA203 | |
| PA204 | 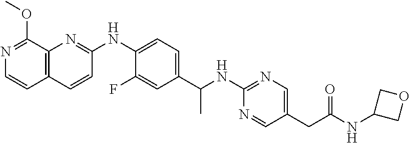 [SMILES: COc1nccc2ccc(Nc3ccc(C(C)Nc4ncc(CC(=O)NC5COC5)cn4)cc3F)nc12] | ¹H NMR (400 MHz, DMSO-d6) δ (ppm) 9.16 (s, 1H), 8.72 (s, 1H), 8.61 (d, 1H), 8.09 (s, 2H), 8.01 (s, 1H), 7.82 (s, 1H), 7.53 (s, 1H), 7.40 (s, 1H), 7.20 (d, 3H), 5.04 (s, 1H), 4.78-4.58 (m, 3H), 4.36 (s, 2H), 3.98 (s, 3H), 3.20 (s, 2H), 1.41 (s, 3H); (ESI) m/z. [M + H]+ 504.0 |
| | PA204 | |
| PA205 | 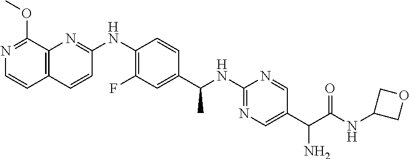 [SMILES: COc1nccc2ccc(Nc3ccc([C@H](C)Nc4ncc(C(N)C(=O)NC5COC5)cn4)cc3F)nc12] | ¹H NMR (400 MHz, DMSO-d6) δ (ppm) 9.18 (s, 1H), 8.70 (t, 1H), 8.65-8.58 (m, 1H), 8.19 (s, 2H), 8.01 (d, 1H), 7.82 (d, 1H), 7.63 (s, 1H), 7.40 (d, 1H), 7.25-7.15 (m, 3H), 5.06 (s, 1H), 4.73 (q, 1H), 4.63 (q, 2H), 4.46-4.31 (m, 2H), 4.14 (s, 1H), 3.97 (s, 3H), 2.41-2.13 (m, 2H), 1.43-1.38 (m, 3H); (ESI) m/z. [M + H]+ 519.40 |
| | PA205 | |
| PA206 | 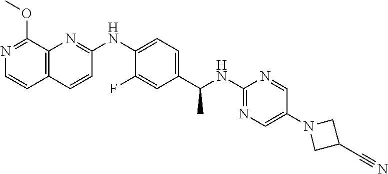 [SMILES: COc1nccc2ccc(Nc3ccc([C@H](C)Nc4ncc(N5CC(C#N)C5)cn4)cc3F)nc12] | ¹H NMR (400 MHz, DMSO-d6) δ (ppm) 9.21 (s, 1H), 8.65-8.56 (m, 1H), 8.02 (d, 1H), 7.82 (d, 1H), 7.77 (s, 2H), 7.40 (dd, 1H), 7.25-7.19 (m, 2H), 7.16 (dd, 1H), 4.94 (q, 1H), 3.97 (d, 5H), 3.85 (td, 2H), 3.78-3.70 (m, 1H), 1.40 (d, 3H). (ESI) m/z. [M + H]⁺ 471.3 |
| | PA206 | |
| PA207 | 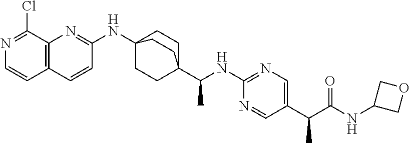 [SMILES: C[C@H](C(=O)NC1COC1)c1cnc(N[C@@H](C)C23CCC(Nc4ccc5ccnc(Cl)c5n4)(CC2)CC3)nc1] | ¹H NMR (400 MHz, DMSO-d6) δ (ppm) 8.76 (d, 1H), 8.15 (s, 2H), 7.95 (d, 1H), 7.86 (d, 1H), 7.53 (d, 1H), 7.18 (s, 1H) , 7.02 (d, 1H), 6.75 (d, 1H), 4.79-4.64 (m, 3H), 4.41 (t, 1H), 4.35 (t, 1H), 3.99-3.90 (m, 1H), 3.41 (q, 1H), 2.10-2.06 (m, 6H), 1.64-1.50 (m, 6H), 1.30 (d, 3H), 1.00 (d, 3H); (ESI) m/z. [M + H]+ 536.0 |
| | PA207 | |
| PA208 | 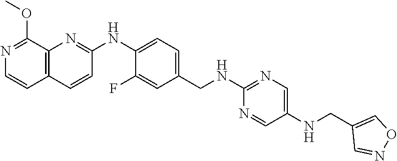 [SMILES: COc1nccc2ccc(Nc3ccc(CNc4ncc(NCc5cnoc5)cn4)cc3F)nc12] | ¹H NMR (400 MHz, DMSO-d6) δ (ppm) 9.20 (s, 1H), 8.85 (s, 1H), 8.66 − 8.60 (m, 1H), 8.56 (s, 1H), 8.04 (d, 1H), 7.88 (d, 2H), 7.86-7.84 (m, 1H), 7.43 (dd, 1H), 7.23 (d, 1H), 7.17-7.11 (m, 2H), 6.97-6.93 (m, 1H), 5.37 (t, 1H), 4.40 (d, 2H), 4.07 (d, 2H), 4.01 (d, 3H); (ESI) m/z. [M + H]+ 473.1 |
| | PA208 | |
| PA209 | 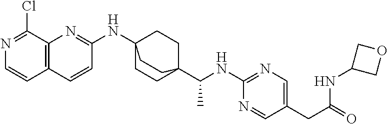 [SMILES: C[C@@H](Nc1ncc(CC(=O)NC2COC2)cn1)C12CCC(Nc3ccc4ccnc(Cl)c4n3)(CC1)CC2] | ¹H NMR (400 MHz, DMSO-d6) δ (ppm) 8.79 (br d, 1H), 8.09 (s, 2H), 7.95 (d, 1H), 7.86 (d, 1H), 7.53 (d, 1H), 7.17 (s, 1H), 7.01 (d, 1H), 6.69 (d, 1H), 4.72-4.81 (m, 1H), 4.67-4.72 (m, 2H), 4.41 (t, 2H), 3.89-4.00 (m, 1H), 3.23 (s, 2H), 2.07 (br t, J = 7.78 Hz, 6H), 1.57 (br d, 6H), 1.01 (d, 3H); (ESI) m/z. [M + H]+ 522.1 |
| | PA209 | |
| PA210 | 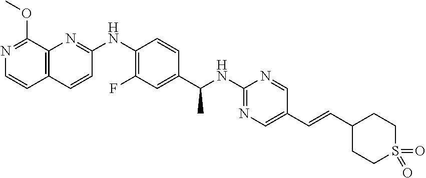 [SMILES: COc1nccc2ccc(Nc3ccc([C@H](C)Nc4ncc(/C=C/C5CCS(=O)(=O)CC5)cn4)cc3F)nc12] | ¹H NMR (400 MHz, DMSO-d6) δ (ppm) 9.19 (s, 1H), 8.62 (t, 1H), 8.34 (s,2H), 8.01 (d, 1H), 7.82 (d, 1H), 7.73 (d, 1H), 7.40 (d, 1H), 7.25-7.14 (m, 3H), 6.23-6.11 (m, 2H), 5.10-5.01 (m, 1H), 3.97 (s, 3H), 3.22-2.97 (m, 4H), 2.01 (dd, 2H), 1.86-1.71 (m, 2H), 1.42 (d, 3H); (ESI) m/z. [M + H]+ 549.3 |
| | PA210 | |
| PA211 | 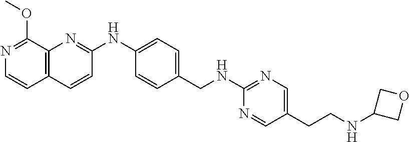 [SMILES: COc1nccc2ccc(Nc3ccc(CNc4ncc(CCNC5COC5)cn4)cc3)nc12] | ¹H NMR (400 MHz, DMSO-d6) δ (ppm) 9.55 (s, 1H), 8.14 (s, 2H), 8.01 (d, 1H), 7.94 (d, 1H), 7.92 (d, 1H), 7.84 (d, 1H), 7.43 (d, 1H), 7.27 (s, 1H), 7.25 (d, 2H), 7.22 (d, 1H), 7.21 (s, 1H), 4.60 (d, 2H), 4.45 (d, 2H), 4.28 (t, 2H), 4.03 (s, 3H), 3.87-3.80 (m, 1H), 2.59 (t, 2H), 2.44 (t, 2H); (ESI) m/z. [M + H]+ 458.1 |
| | PA211 | |
| PA212 | 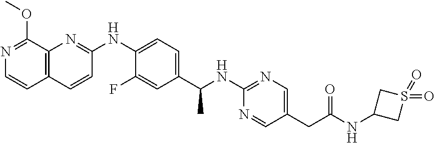 [SMILES: COc1nccc2ccc(Nc3ccc([C@H](C)Nc4ncc(CC(=O)NC5CS(=O)(=O)C5)cn4)cc3F)nc12] | ¹H NMR (400 MHz, DMSO-d6) δ (ppm) 9.21 (s, 1H), 8.80 (d, 1H), 8.64 (t, 1H), 8.13 (s, 2H), 8.04 (d, 1H), 7.85 (d, 1H), 7.59 (d, 1H), 7.44 (dd, 1H), 7.29-7.18 (m, 3H), 5.07 (p, 1H), 4.49 (dd, 2H), 4.32 (tq, 1H), 4.08-4.02 (m, 2H), 4.01 (s, 3H), 3.26 (s, 2H), 1.45 (d, 3H). (ESI) m/z. [M + H]⁺ 552.10 |
| | PA212 | |
| PA213 | 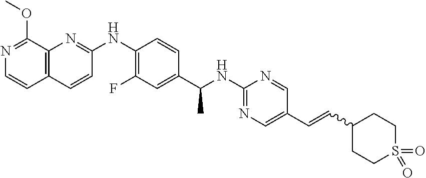 [SMILES: COc1nccc2ccc(Nc3ccc([C@H](C)Nc4ncc(C=CC5CCS(=O)(=O)CC5)cn4)cc3F)nc12] | ¹H NMR (400 MHz, DMSO-d6) δ (ppm) 9.20 (s, 1H), 8.63 (t, 1H), 8.24 (s, 2H), 8.01 (d, 1H), 7.84-7.79 (m, 2H), 7.40 (d, 1H), 7.26-7.16 (m, 3H), 6.07 (d, 1H), 5.39 (dd, 1H), 5.09 (p, 1H), 3.97 (s, 3H), 3.23 (dd, 2H), 2.95 (s, 2H), 2.89-2.78 (m, 1H), 1.98-1.88 (m, 2H), 1.85- 1.71 (m, 2H), 1.44 (d, 3H). (ESI) m/z. [M + H]⁺ 549.3 |
| | PA213 | |
| PA214 | 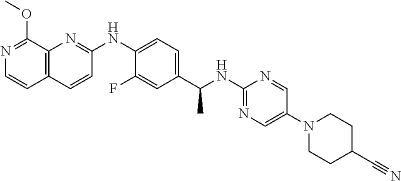 [SMILES: COc1nccc2ccc(Nc3ccc([C@H](C)Nc4ncc(N5CCC(C#N)CC5)cn4)cc3F)nc12] | ¹H NMR (400 MHz, DMSO-d6) δ (ppm) 9.19 (s, 1H), 8.59 (t, 1H), 8.41 (s,3H), 8.08-7.99 (m, 3H), 7.81 (d, 1H), 7.39 (d, 1H), 7.26-7.14 (m, 4H), 5.00-4.91 (m, 1H), 3.97 (s, 3H), 3.13-2.77 (m, 4H), 1.99-1.87 (m, 3H), 1.82-1.69 (m, 2H), 1.39 (d, 3H). (ESI) m/z. [M + H]⁺ 499.4 |
| | PA214 | |
| PA215 | 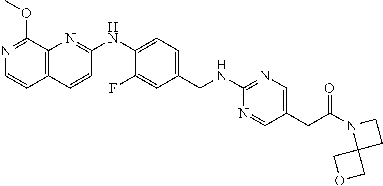 [SMILES: COc1nccc2ccc(Nc3ccc(CNc4ncc(CC(=O)N5CCC56COC6)cn4)cc3F)nc12] | ¹H NMR (400 MHz, DMSO-d6) δ (ppm) 9.23 (d, 1H), 8.66 (t, 1H), 8.22-8.16 (m, 2H), 8.05 (d, 1H), 7.85 (d, 1H), 7.63 (t, 1H), 7.44 (d, 1H), 7.24 (d, 1H), 7.21-7.13 (m, 2H), 5.09 (dd, 2H), 4.83-4.46 (m, 4H), 4.01 (s, 3H) , 3.99 (d, 2H), 3.23 (s, 2H), 2.48-2.41(m, 2H). (ESI) m/z. [M + H]⁺ 516.2 |
| | PA215 | |
| PA216 | 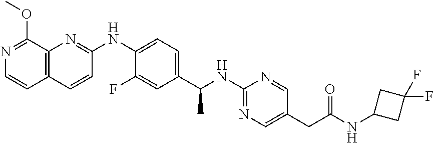 [SMILES: COc1nccc2ccc(Nc3ccc([C@H](C)Nc4ncc(CC(=O)NC5CC(F)(F)C5)cn4)cc3F)nc12] | ¹H NMR (400 MHz, DMSO-d6) δ (ppm) 9.21 (s, 1H), 8.64 (t, 1H), 8.47 (d, 1H), 8.12 (s, 2H), 8.04 (d, 1H), 7.85 (d, 1H), 7.59 (d, 1H), 7.44 (dd, 1H), 7.29-7.18 (m, 3H), 5.11-5.03 (m, 1H), 4.01-3.98 (m, 4H), 3.21 (s, 2H), 2.92-2.81 (m, 2H), 2.60-2.51 (m, 2H), 1.45 (d, 3H); (ESI) m/z. [M + H]+ 538.20 |
| | PA216 | |
| PA217 | 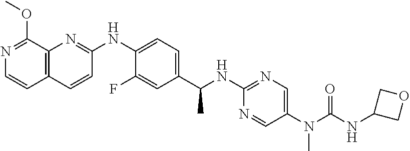 [SMILES: COc1nccc2ccc(Nc3ccc([C@H](C)Nc4ncc(N(C)C(=O)NC5COC5)cn4)cc3F)nc12] | ¹H NMR (400 MHz, DMSO-d6) δ (ppm) 9.19 (s, 1H), 8.62 (t, 1H), 8.14 (s, 2H), 8.01 (d, 1H), 7.82 (d, 2H), 7.41 (dd, 1H), 7.27 (dd, 1H), 7.23-7.19 (m, 2H), 6.80 (d, 1H), 5.11-5.03 (m, 1H), 4.68-4.60 (m, 1H), 4.53 (t, 2H), 4.38 (t, 2H), 3.97 (s, 3H), 3.00 (s, 3H), 1.43 (d, 3H); (ESI) m/z. [M + H]+ 519.2 |
| | PA217 | |
| PA218 | 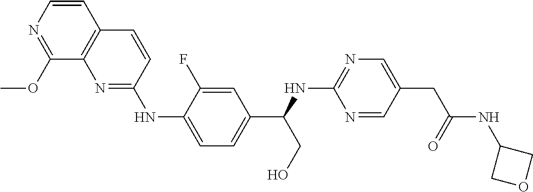 [SMILES: COc1nccc2ccc(Nc3ccc([C@H](CO)Nc4ncc(CC(=O)NC5COC5)cn4)cc3F)nc12] | ¹H NMR (400 MHz, DMSO-d6) δ (ppm) 9.18 (s, 1H), 8.73 (d, 1H), 8.67-8.57 (m, 1H), 8.09 (s, 2H), 8.01 (d, 1H), 7.82 (d, 1H), 7.41(dd, 1H), 7.31 (d, 1H), 7.25-7.14 (m, 3H), 5.00-4.92 (m, 1H), 4.70 (s, 1H), 4.67-4.61 (m, 2H), 4.36 (t, 2H), 3.97 (s, 3H), 3.66-3.56 (m, 2H), 3.20 (s, 2H); (ESI) m/z. [M + H]+ 520.3 |
| | PA218 | |
| PA219 | 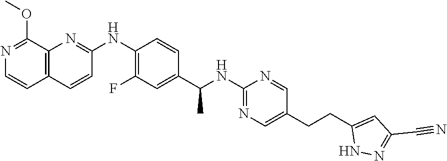 [SMILES: COc1nccc2ccc(Nc3ccc([C@H](C)Nc4ncc(CCc5cc(C#N)n[nH]5)cn4)cc3F)nc12] | ¹H NMR (400 MHz, DMSO-d6) δ (ppm) 13.67 (s, 1H), 9.19 (s, 1H), 8.59 (t, 1H), 8.05 (s, 2H), 8.01 (d, 1H), 7.81 (d, 1H), 7.50 (d, 1H), 7.39 (d, 1H), 7.24-7.19 (m, 2H), 7.16 (dd, 1H), 6.68 (s, 1H), 5.01 (p, 1H), 3.97 (s, 3H), 2.83 (t, 2H), 2.66 (t, 2H), 1.40 (d, 3H); (ESI) m/z. [M + H]+ 510.3 |
| | PA219 | |
| PA220 | 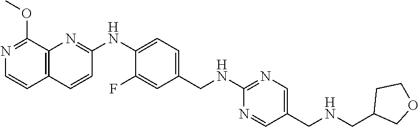 [SMILES: COc1nccc2ccc(Nc3ccc(CNc4ncc(CNCC5CCOC5)cn4)cc3F)nc12] | ¹H NMR (400 MHz, DMSO-d6) δ (ppm) 9.22 (s, 1H), 8.65 (t, 1H), 8.22 (s, 2H), 8.05 (d, 1H), 7.85 (d, 1H), 7.60 (t, 1H), 7.44 (d, 1H), 7.23 (d, 1H), 7.20-7.13 (m, 2H), 4.48 (d, 2H), 4.01 (s, 3H), 3.72-3.63 (m, 2H), 3.60-3.54 (m, 1H), 3.48 (s, 2H), 3.37-3.31 (m, 2H), 2.48-2.37 (m, 2H), 2.32-2.21 (m, 1H), 1.95-1.86 (m, 1H), 1.53-1.42 (m, 1H). (ESI) m/z. [M + H]⁺ 490.1 |
| | PA220 | |
| PA221 | 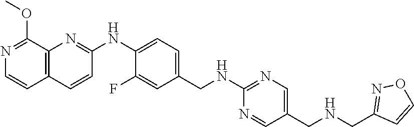 [SMILES: COc1nccc2ccc(Nc3ccc(CNc4ncc(CNCc5ccon5)cn4)cc3F)nc12] | ¹H NMR (400 MHz, DMSO-d6) δ (ppm) 9.17 (d, 1H), 8.77 (d, 1H), 8.64-8.57 (m, 1H), 8.19 (s, 2H), 8.01 (d, 1H), 7.82 (d, 1H), 7.58 (t, 1H), 7.40 (dd, 1H), 7.20 (d, 1H), 7.16-7.08 (m, 2H), 6.54 (d, 1H), 4.45 (d, 2H), 3.97 (s, 3H), 3.69 (s, 2H), 3.44 (s, 2H). (ESI) m/z. [M + H]+ 487.3 |
| | PA221 | |
| PA222 | 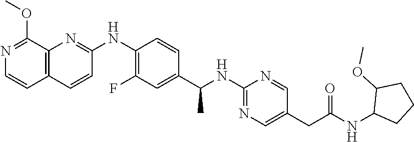 [SMILES: COc1nccc2ccc(Nc3ccc([C@H](C)Nc4ncc(CC(=O)NC5CCCC5OC)cn4)cc3F)nc12] | ¹H NMR (400 MHz, DMSO-d6) δ (ppm) 9.22 (d, 1H), 8.65 (t, 1H), 8.12 (s, 2H), 8.03 (t, 2H), 7.85 (d, 1H), 7.57 (d, 1H), 7.44 (d, 1H), 7.28-7.23 (m, 2H), 7.20 (dd, 1H), 5.11 −5.04 (m, 1H), 4.01 (s, 3H), 3.92-3.85 (m, 1H), 3.50-3.46 (m, 1H), 3.18 (s, 2H), 3.17 (d, 3H), 1.90-1.74 (m, 2H), 1.66-1.50 (m, 3H), 1.45 (d, 3H), 1.38-1.30 (m, 1H); (ESI) m/z. [M + H]+ 546.1 |
| | PA222 | |
| PA223 | 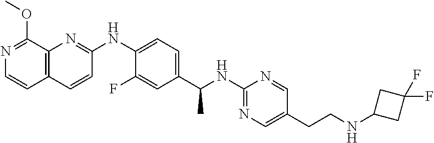 [SMILES: COc1nccc2ccc(Nc3ccc([C@H](C)Nc4ncc(CCNC5CC(F)(F)C5)cn4)cc3F)nc12] | ¹H NMR (400 MHz, DMSO-d6) δ (ppm) 9.19 (s, 1H), 8.63 (t, 1H), 8.14 (s, 2H), 8.04 (d, 1H), 7.85 (d, 1H), 7.44 (t, 2H), 7.30-7.17 (m, 3H), 5.15-4.95 (m, 1H), 4.01 (s, 3H), 3.12 (d, 1H), 2.76-2.64 (m, 2H), 2.59 (t, 2H), 2.45 (t, 2H), 2.37-2.21 (m, 2H), 1.44 (d, 3H); (ESI) m/z. [M + H]+ 524.2 |
| | PA223 | |
| PA224 | 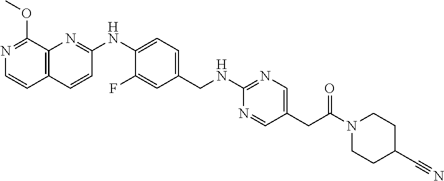 [SMILES: COc1nccc2ccc(Nc3ccc(CNc4ncc(CC(=O)N5CCC(C#N)CC5)cn4)cc3F)nc12] | ¹H NMR (400 MHz, DMSO-d6) δ (ppm) 9.19 (s, 1H), 8.68-8.59 (m, 1H), 8.08 (s, 2H), 8.01 (d, 1H), 7.82 (d, 1H), 7.56 (t, 1H), 7.41 (dd, 1H), 7.20 (d, 1H), 7.17-7.09 (m, 2H), 4.45 (d, 2H), 3.97 (s, 3H), 3.80-3.62 (m, 2H), 3.50 (s, 2H), 3.36 (d, 1H), 3.20-3.12 (m, 1H), 3.11-3.02 (m, 1H), 1.90-1.75 (m, 2H), 1.73-1.52 (m, 2H); (ESI) m/z. [M + H]+ 527.0 |
| | PA224 | |
| PA225 | 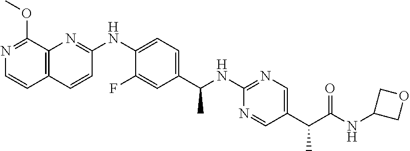 [SMILES: COc1nccc2ccc(Nc3ccc([C@H](C)Nc4ncc([C@@H](C)C(=O)NC5COC5)cn4)cc3F)nc12] | ¹H NMR (400 MHz, DMSO-d6) δ (ppm) 9.16 (s, 1H), 8.69-8.57 (m, 2H), 8.14 (s, 2H), 8.01 (d, 1H), 7.82 (d, 1H),7.56 (d, 1H), 7.40 (d, 1H), 7.25-7.15 (m, 3H), 5.08-4.99 (m, 1H), 4.71-4.62 (m, 2H),4.59 (d, 1H), 4.32 (dt, 2H), 3.97 (s, 3H), 3.37 (d, 1H), 1.41 (d, 3H), 1.24 (d, 3H); (ESI) m/z. [M + H]+ 518.2 |
| | PA225 | |
| PA226 | 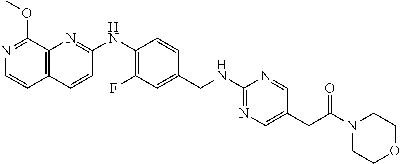 [SMILES: COc1nccc2ccc(Nc3ccc(CNc4ncc(CC(=O)N5CCOCC5)cn4)cc3F)nc12] | ¹H NMR (400 MHz, DMSO-d6) δ (ppm) 9.22 (s, 1H) , 8.67 (dt, 1H), 8.12 (s, 2H), 8.05 (dd, 1H), 7.85 (dd, 1H), 7.59 (t, 1H), 7.44 (dd, 1H), 7.24 (d, 1H), 7.20-7.13 (m, 2H) , 4.49 (d, 2H), 4.01 (d, 3H), 3.60-3.56 (m, 2H), 3.54-3.53 (m, 6H ), 3.46-3.42 (m, 2H); (ESI) m/z. [M + H]+ 504.0 |
| | PA226 | |
| PA227 | 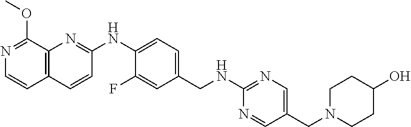 [SMILES: COc1nccc2ccc(Nc3ccc(CNc4ncc(CN5CCC(O)CC5)cn4)cc3F)nc12] | ¹H NMR (400MHz, DMSO-d6) δ (ppm) 9.12 (s, 1H), 8.56 (t, 1H), 8.10 (s, 2H), 8.05 (s, 1H), 7.95 (d, 1H), 7.75 (d, 1H), 7.61 (br t, 1H), 7.34 (d, 1H), 7.13 (d, 1H), 7.11-7.02 (m, 2H), 4.39 (br d, 2H), 3.91 (s, 3H), 3.26 (br s, 3H), 2.67-2.55 (m, 2H), 2.03 (br s, 2H), 1.60 (br d, 2H), 1.29 (q, 2H); (ESI) m/z. [M + H]+ 490.1 |
| | PA227 | |
| PA228 | 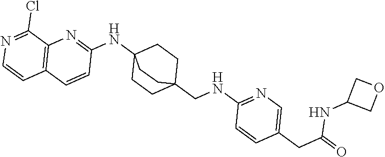 [SMILES: O=C(Cc1ccc(NCC23CCC(Nc4ccc5ccnc(Cl)c5n4)(CC2)CC3)nc1)NC1COC1] | ¹H NMR (400 MHz, DMSO-d₆) δ (ppm) 8.74 (d, 1H), 7.95 (d, 1H), 7.86 (d, 1H), 7.76 (d, 1H), 7.53 (d, 1H), 7.23 (dd, 1H), 7.17 (s, 1H), 7.02 (d, 1H), 6.49 (d, 1H), 6.26 (m, 1H), 4.70-4.82 (m, 1H), 4.69 (t, 2H), 4.39 (t, 2H), 3.20 (s, 2H), 3.06 (d, 2H), 2.02-2.16 (m, 6H), 1.46-1.64 (m, 6H); (ESI) m/z = 507.1 |
| | PA228 | |
| PA229 | 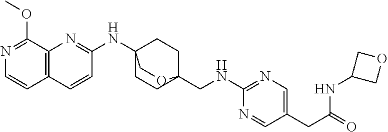 [SMILES: COc1nccc2ccc(NC34CCC(CNc5ncc(CC(=O)NC6COC6)cn5)(CC3)OC4)nc12] | ¹H NMR (400 MHz, DMSO-d6) δ (ppm) 8.80 (br d, 1H), 8.13 (s, 2H), 7.78 (d, 1H), 7.71 (d, 1H), 7.09 (d, 1H), 6.94 (d, 1H), 6.85 (s, 1H), 6.75 (t, 1H), 4.72-4.82 (m, 1H), 4.67-4.72 (m, 2H), 4.41 (t, 2H), 4.18 (s, 2H), 3.98 (s, 3H), 3.32 (br s, 2H), 3.25 (s, 2H), 2.28-2.40 (m, 2H), 1.80-1.97 (m, 4H), 1.67-1.79 (m, 2H); (ESI) m/z. [M + H]+ 506.2 |
| | PA229 | |
| PA230 | 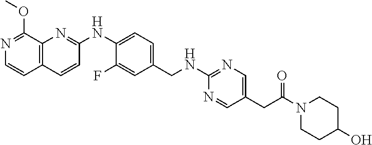 [SMILES: COc1nccc2ccc(Nc3ccc(CNc4ncc(CC(=O)N5CCC(O)CC5)cn4)cc3F)nc12] | ¹H NMR (400 MHz, DMSO-d6) δ (ppm) 9.22 (s, 1H), 8.66 (q, 1H), 8.12 (s, 2H), 8.05 (d, 1H), 7.85 (d, 1H), 7.58 (t, 1H), 7.44 (dd, 1H), 7.23 (d, 1H), 7.21-7.11 (m, 2H), 4.73 (d, 1H), 4.48 (d, 2H), 4.01 (s, 3H), 3.89 (d, 1H), 3.77 (d, 1H), 3.68 (tt, 1H), 3.52 (s, 2H), 3.21 (t, 1H), 3.00 (t, 1H), 1.78-1.61 (m, 2H), 1.27 (dddd, 2H); (ESI) m/z. [M + H]+ 518.20 |
| | PA230 | |
| PA231 | 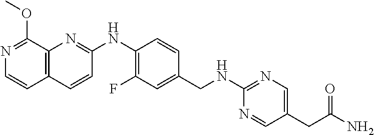 [SMILES: COc1nccc2ccc(Nc3ccc(CNc4ncc(CC(N)=O)cn4)cc3F)nc12] | ¹H NMR (400MHz, DMSO-d6) δ (ppm) 9.21 (s, 1H), 8.64 (t, 1H), 8.16 (s, 2H), 8.05 (d, 1H), 7.86 (d, 1H), 7.59 (t, 1H), 7.44 (d, 2H), 7.24 (d, 1H), 7.21-7.12 (m, 2H), 6.90 (br s, 1H), 4.49 (d, 2H), 4.01 (s, 3H), 3.25-3.14 (m, 2H); (ESI) m/z. [M + H]+ 434.3 |
| | PA231 | |
| PA232 | 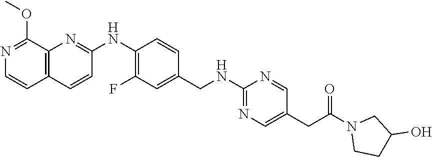 [SMILES: COc1nccc2ccc(Nc3ccc(CNc4ncc(CC(=O)N5CCC(O)C5)cn4)cc3F)nc12] | ¹H NMR (400 MHz, Chloroform-d) δ (ppm) 8.22 (s, 2H), 8.10 (t, 1H), 7.92 (dd, 2H), 7.22-7.08 (m, 4H), 5.75 (s, 1H), 4.59 (dd, 3H), 4.20 (s, 3H), 3.78-3.58 (m, 3H), 3.57-3.40 (m, 3H), 2.09 (s, 2H); (ESI) m/z. [M + H]+ 504.2 |
| | PA232 | |
| PA233 | 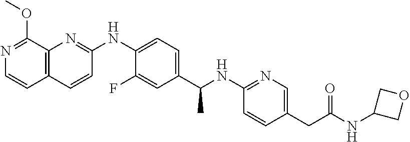 [SMILES: COc1nccc2ccc(Nc3ccc([C@H](C)Nc4ccc(CC(=O)NC5COC5)cn4)cc3F)nc12] | ¹H NMR (400 MHz, DMSO-d6) δ (ppm) 9.20 (d, 1H), 8.71 (d, 1H), 8.65 (t, 1H), 8.04 (d, 1H), 7.85 (d, 1H), 7.78 (d, 1H), 7.44 (dd, 1H), 7.29-7.17 (m, 4H), 6.86 (d, 1H), 6.45 (d, 1H), 4.98 (p, 1H), 4.79-4.69 (m, 1H), 4.67 (t, 2H), 4.38 (t, 2H), 4.01 (s, 3H), 3.20 (s, 2H), 1.43 (d, 3H); (ESI) m/z. [M + H]+ 503.20 |
| | PA233 | |
| PA234 | 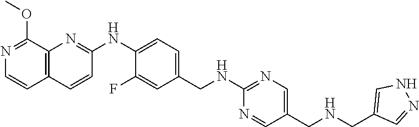 [SMILES: COc1nccc2ccc(Nc3ccc(CNc4ncc(CNCc5cn[nH]c5)cn4)cc3F)nc12] | ¹H NMR (400 MHz, DMSO-d6) δ (ppm) 9.19 (s, 1H), 8.62 (d, 1H), 8.20 (s, 2H), 8.01 (d, 1H), 7.81 (s, 1H), 7.61-7.54 (m, 1H), 7.53-7.37 (m, 3H), 7.20 (s, 1H), 7.13 (t, 2H), 4.44 (s, 2H), 3.96 (s, 3H), 3.50 (s, 2H), 3.44 (s, 2H); (ESI) m/z. [M + H]+ 486.0 |
| | PA234 | |
| PA235 | 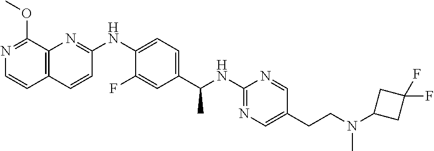 [SMILES: COc1nccc2ccc(Nc3ccc([C@H](C)Nc4ncc(CCN(C)C5CC(F)(F)C5)cn4)cc3F)nc12] | ¹H NMR NMR (400 MHz, DMSO-d6) 8 (ppm) 9.19 (s, 1H), 8.69-8.61 (m, 1H), 8.15 (s, 2H), 8.04 (dd, 1H), 7.85 (dd, 1H), 7.44 (dd, 2H), 7.27-7.18 (m, 3H), 5.12-5.01 (m, 1H), 4.01 (s, 3H), 2.78-2.69 (m, 1H), 2.68-2.56 (m, 2H), 2.48-2.44 (m, 2H), 2.38 (d, 2H), 2.32-2.22 (m, 2H), 2.12 (s, 3H), 1.44 (d, 3H); (ESI) m/z. [M + H]+ 538.2 |
| | PA235 | |
| PA236 | 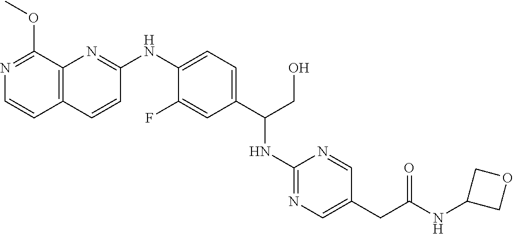 [SMILES: COc1nccc2ccc(Nc3ccc(C(CO)Nc4ncc(CC(=O)NC5COC5)cn4)cc3F)nc12] | ¹H NMR (400 MHz, DMSO-d6) δ (ppm) 9.18 (s, 1H), 8.73 (d, 1H), 8.67-8.57 (m, 1H), 8.09 (s, 2H), 8.01 (d, 1H), 7.82 (d, 1H), 7.41(dd, 1H), 7.31 (d, 1H), 7.25-7.14 (m, 3H), 5.00-4.92 (m, 1H), 4.89-4.82 (m, 1H), 4.70 (s, 1H), 4.67-4.61 (m, 2H), 4.36 (t, 2H), 3.97 (s, 3H), 3.66-3.56 (m, 2H), 3.20 (s, 2H); (ESI) m/z. [M + H]+ 520.1 |
| | PA236 | |
| PA237 | 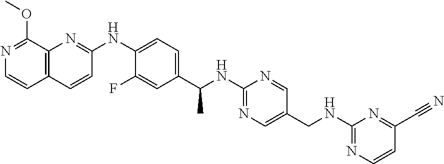 | ¹H NMR (400 MHz, DMSO-d6) δ (ppm) 9.21-9.18 (m, 1H), 8.60 (t, 1H), 8.41 (s, 2H), 8.14 (d, 2H), 8.01 (d, 1H), 7.82 (d, 1H), 7.54 (d, 1H), 7.40 (d, 1H), 7.25-7.14 (m, 3H), 5.08-4.99 (m, 1H), 3.97 (s, 3H), 3.03 (t, 1H), 2.73 (d, 1H), 2.43-2.38 (m, 2H), 2.32-2.27 (m, 1H), 1.95-1.88 (m, 2H), 1.41 (d, 3H); (ESI) m/z. [M + H]+ 499.3 |
| | PA237 | |
| PA238 | 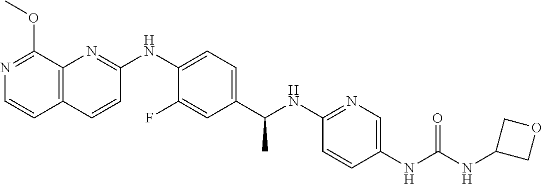 [SMILES: COc1nccc2ccc(Nc3ccc([C@H](C)Nc4ccc(NC(=O)NC5COC5)cn4)cc3F)nc12] | ¹H NMR (400 MHz, DMSO-d6) δ (ppm) 9.15 (s, 1H), 8.68-8.59 (m, 1H), 8.00 (d, 1H), 7.96 (s, 1H), 7.85-7.80 (m, 2H), 7.44-7.33 (m, 2H), 7.23-7.14 (m, 3H), 6.78-6.60 (m, 1H), 6.41 (d, 1H), 4.94-4.86 (m, 1H), 4.72-4.61 (m, 3H), 4.36 (t, 2H), 3.98 (s, 3H), 1.39 (d, 3H); (ESI) m/z. [M + H]+ 504.1 |
| | PA238 | |
| PA239 | 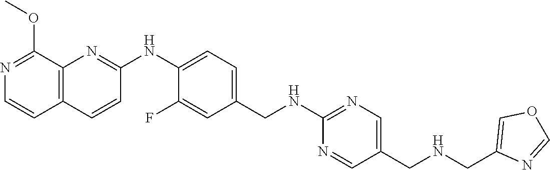 [SMILES: COc1nccc2ccc(Nc3ccc(CNc4ncc(CNCc5cocn5)cn4)cc3F)nc12] | ¹H NMR (400 MHz, DMSO-d6) δ (ppm) 9.21 (s, 1H), 8.65 (t, 1H), 8.28 (d, 1H), 8.24 (s, 2H), 8.04 (d, 1H), 7.92 (d, 1H), 7.85 (d, 1H), 7.60 (t, 1H), 7.44 (dd, 1H), 7.23 (d, 1H), 7.21-7.11 (m, 2H), 4.49 (d, 2H), 4.00 (s, 3H), 3.54 (d, 4H); (ESI) m/z. [M + H]+ 487.30 |
| | PA239 | |
| PA240 | 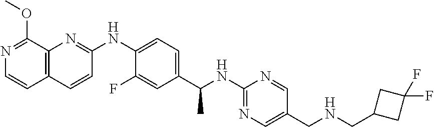 [SMILES: COc1nccc2ccc(Nc3ccc([C@H](C)Nc4ncc(CNCC5CC(F)(F)C5)cn4)cc3F)nc12] | ¹H NMR (400 MHz, DMSO-d6) δ (ppm) 9.18 (s, 1H), 8.61 (t, 1H), 8.17 (s, 2H), 8.01 (d, 1H), 7.82 (d, 1H), 7.56 (s, 1H), 7.40 (dd, 1H), 7.25-7.15 (m, 3H), 5.05 (t, 1H), 3.97 (s, 3H), 3.47 (s, 2H), 2.53 (d, 3H), 2.32-2.09 (m, 4H), 1.41 (d, 3H). (ESI) m/z. [M + H]⁺ 524.0 |
| | PA240 | |
| PA241 | 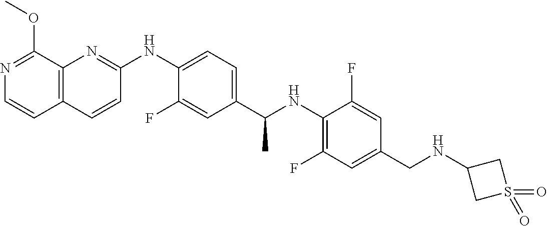 [SMILES: COc1nccc2ccc(Nc3ccc([C@H](C)Nc4c(F)cc(CNC5CS(=O)(=O)C5)cc4F)cc3F)nc12] | ¹H NMR (400 MHz, DMSO-d6) δ (ppm) 9.20 (s, 1H), 8.70 (t, 1H), 8.04 (d, 1H), 7.86 (d, 1H), 7.44 (d, 1H), 7.29-7.21 (m, 2H), 7.14 (dd, 1H), 6.86 (d, 2H), 5.26 (d, 1H), 4.77-4.65 (m, 1H), 4.27-4.19 (m, 2H), 4.01 (s, 3H), 3.87-3.80 (m, 2H), 3.45 (s, 2H), 3.443.38 (m, 1H), 1.49 (d, 3H); (ESI) m/z = 558.30 |
| | PA241 | |
| PA242 | 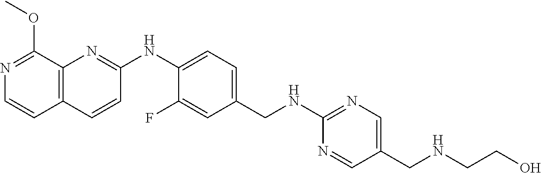 [SMILES: COc1nccc2ccc(Nc3ccc(CNc4ncc(CNCCO)cn4)cc3F)nc12] | ¹H NMR (400 MHz, DMSO-d6) δ (ppm) 9.24 (s, 1H), 8.66 (t, 1H), 8.30 (s, 2H), 8.05 (d, 1H), 7.86 (d, 1H), 7.78 (t, 1H), 7.44 (d, 1H), 7.24 (d, 1H), 7.16 (d, 2H), 4.77 (s, 1H), 4.56-4.43 (m, 2H), 4.01 (s, 3H), 3.70 (s, 2H), 3.52 (s, 2H), 2.71 (s, 2H); (ESI) m/z. [M + H]+ 450.10 |
| | PA242 | |
| PA243 | 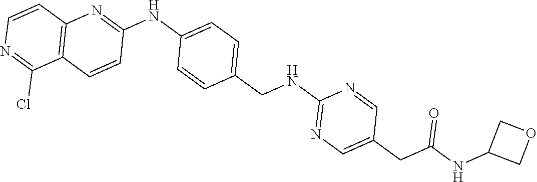 [SMILES: O=C(Cc1cnc(NCc2ccc(Nc3ccc4c(Cl)nccc4n3)cc2)nc1)NC1COC1] | ¹H NMR (400 MHz, DMSO-d6) δ (ppm) 9.92 (s, 1H), 8.76 (s, 1H), 8.21 (d, 2H), 8.11 (s, 2H), 7.84-7.77 (m, 2H), 7.55 (s, 1H), 7.48 (s, 1H), 7.30-7.24 (m, 2H), 7.17 (d, 1H), 4.77-4.69 (m, 1H), 4.64 (s, 2H), 4.43 (s, 2H), 4.37 (t, 2H), 3.22 (s, 2H); (ESI) m/z. [M + H]+ 476.0 |
| | PA243 | |
| PA244 | 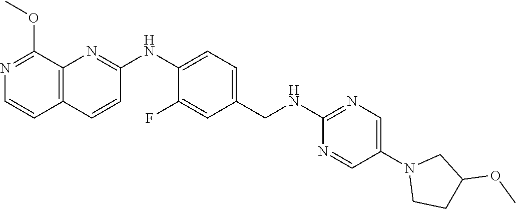 [SMILES: COc1nccc2ccc(Nc3ccc(CNc4ncc(N5CCC(OC)C5)cn4)cc3F)nc12] | ¹H NMR (400 MHz, DMSO-d6) d 9.22 (s, 1H), 8.63 (t, 1H), 8.05 (d, 1H), 7.86 (s, 1H), 7.85 (s, 2H), 7.43 (d, 1H), 7.24 (d, 1H), 7.20-7.12 (m, 2H), 6.97 (t, 1H), 4.42 (br d, 2H), 4.05 (br s, 1H), 4.01 (s, 3H), 3.24 (s, 3H), 3.20-3.13 (m, 4H), 2.11-1.91 (m, 2H); (ESI) m/z. [M + H]+ 476.0 |
| | PA244 | |
| PA245 | 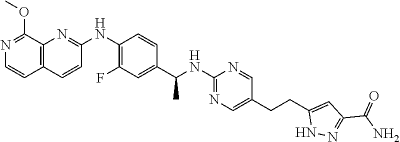 [SMILES: COc1nccc2ccc(Nc3ccc([C@H](C)Nc4ncc(CCc5cc(C(N)=O)n[nH]5)cn4)cc3F)nc12] | ¹H NMR (400 MHz, DMSO-d6) δ (ppm) 9.29 (s, 1H), 8.72-8.63 (m, 1H), 8.25 (s, 2H), 8.08 (d, 1H), 7.88 (d, 1H), 7.45 (d, 2H), 7.33-7.14 (m, 4H), 6.46 (s, 1H), 5.09 (s, 1H), 4.02 (s, 3H), 2.91-2.69 (m, 4H), 1.49 (d, 3H); (ESI) m/z. [M + H]+ 528.3 |
| | PA245 | |
| PA246 | 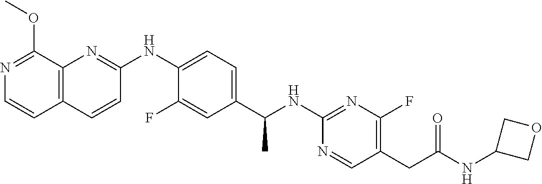 [SMILES: COc1nccc2ccc(Nc3ccc([C@H](C)Nc4ncc(CC(=O)NC5COC5)c(F)n4)cc3F)nc12] | ¹H NMR (400 MHz, DMSO-d6) δ (ppm) 9.23 (s, 1H), 8.80-8.64 (m, 2H), 8.05 (d, 1H), 7.86 (d, 1H), 7.45 (d, 1H), 7.38 (dd, 1H), 7.31-7.18 (m, 4H), 6.34 (dd, 1H), 4.87 (p, 1H), 4.75 (dq, 1H), 4.70-4.64 (m, 2H), 4.39 (t, 2H), 4.01 (s, 3H), 3.24 (s, 2H), 1.43 (d, 3H); (ESI) m/z. [M + H]+ 521.3 |
| | PA246 | |
| PA247 | 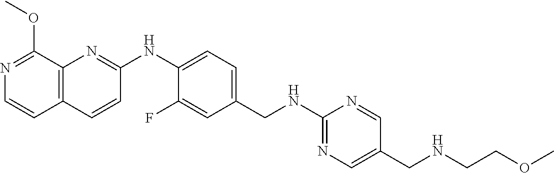 [SMILES: COCCNCc1cnc(NCc2ccc(Nc3ccc4ccnc(OC)c4n3)c(F)c2)nc1] | ¹H NMR (400 MHz, DMSO-d6) δ (ppm) 9.22 (s, 1H), 8.71-8.61 (m, 1H), 8.24 (d, 3H), 8.04 (d, 1H), 7.85 (d, 1H), 7.70 (t, 1H), 7.47-7.40 (m, 1H), 7.23 (d, 1H), 7.20-7.11 (m, 2H), 4.48(d, 2H), 4.00 (s, 3H), 3.61 (s, 2H), 3.40 (d, 2H), 3.23 (s,3H), 2.72 (t, 2H). (ESI) m/z. [M + H]⁺ 464.1 |
| | PA247 | |
| PA248 | 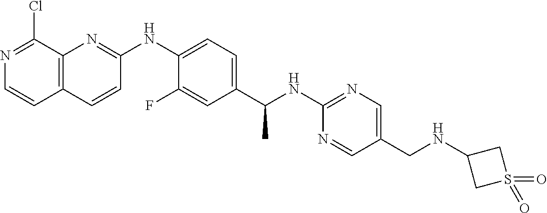 [SMILES: C[C@H](Nc1ncc(CNC2CS(=O)(=O)C2)cn1)c1ccc(Nc2ccc3ccnc(Cl)c3n2)c(F)c1] | ¹H NMR (400 MHz, DMSO-d6) δ (ppm) 8.99 (d, 1H), 8.36 (d, 1H), 8.21-8.13 (m, 3H), 8.04 (d, 1H), 7.82 (d, 1H), 7.66 (d, 1H), 7.26 (dd, 1H), 7.23-7.16 (m, 2H), 5.06 (p, 1H), 4.32-4.21 (m, 2H), 3.88 (d, 2H), 3.43 (d, 3H), 1.42 (d, 3H); (ESI) m/z. [M + H]+ 528.2 |
| | PA248 | |
| PA249 | 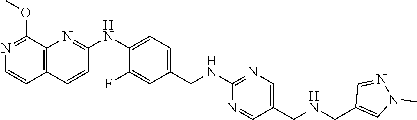 [SMILES: COc1nccc2ccc(Nc3ccc(CNc4ncc(CNCc5cnn(C)c5)cn4)cc3F)nc12] | ¹H NMR (400 MHz, DMSO-d6) δ (ppm) 9.24 (s, 1H), 8.65 (t, 1H), 8.30 (s, 2H), 8.05 (d, 1H), 7.85 (d, 1H), 7.77 (t, 1H), 7.66 (s, 1H), 7.44 (d, 1H), 7.41 (s, 1H), 7.24 (d, 1H), 7.21-7.11 (m, 2H), 4.49 (d, 2H), 4.00 (s, 3H), 3.79 (s, 3H), 3.68 (d, 4H); (ESI) m/z. [M + H]+ 500.20 |
| | PA249 | |
| PA250 | 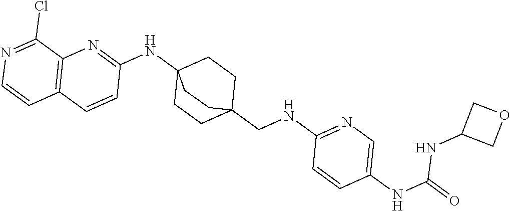 [SMILES: O=C(Nc1ccc(NCC23CCC(Nc4ccc5ccnc(Cl)c5n4)(CC2)CC3)nc1)NC1COC1] | ¹H NMR (400 MHz, DMSO-d₆) δ (ppm) 7.99 (s, 1H), 7.95 (d, 1H), 7.86 (d, 1H), 7.84 (d, 1H), 7.53 (d, 1H), 7.35 (dd, 1H), 7.16 (s, 1H), 7.02 (d, 1H), 6.80 (d, 1H), 6.47 (d, 1H), 6.06 (t, 1H), 4.71-4.80 (m, 1H), 4.65-4.71 (m, 2H), 4.41 (t, 2H), 3.04 (d, 2H), 1.99-2.19 (m, 6H), 1.46-1.67 (m, 6H); (ESI) m/z. [M + H]+ 508.1 |
| | PA250 | |
| PA251 | 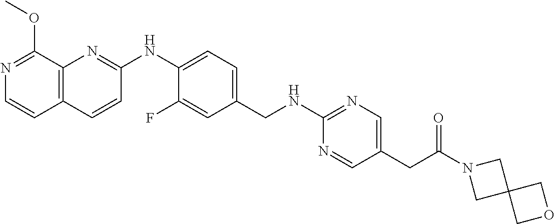 [SMILES: COc1nccc2ccc(Nc3ccc(CNc4ncc(CC(=O)N5CC6(COC6)C5)cn4)cc3F)nc12] | ¹H NMR (400 MHz, DMSO-d6) δ (ppm) 9.22 (s, 1H), 8.72-8.60 (m, 1H), 8.13 (s, 2H), 8.05 (d, 1H), 7.85 (d, 1H), 7.61 (t, 1H), 7.44 (dd, 1H), 7.24 (d, 1H), 7.20-7.10 (m, 2H), 4.72-4.60 (m, 4H), 4.48 (d, 2H), 4.36 (s, 2H), 4.01 (d, 5H), 3.21 (s, 2H); (ESI) m/z. [M + H]+ 516.20 |
| | PA251 | |
| PA252 | 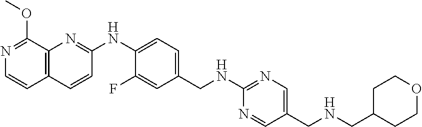 [SMILES: COc1nccc2ccc(Nc3ccc(CNc4ncc(CNCC5CCOCC5)cn4)cc3F)nc12] PA252 | ¹H NMR (400 MHz, DMSO-d6) δ (ppm) 9.22 (s, 1H), 8.69-8.61 (m, 1H), 8.34 (s, 2H), 8.07-8.01 (m, 1H), 7.96 (s, 1H), 7.85 (d, 1H), 7.43 (d, 1H), 7.23 (d, 1H), 7.21-7.10 (m, 2H), 4.50 (d, 2H), 4.00(s, 3H), 3.93-3.79 (m, 4H), 3.23 (d, 2H), 2.74 (s, 2H), 1.82 (s, 1H), 1.60 (d, 2H), 1.27-1.10 (m, 2H). (ESI) m/z. [M + H]⁺ 504.2 |
| PA253 | 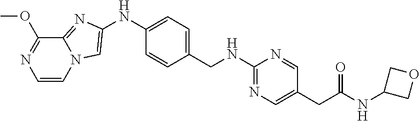 [SMILES: COc1nccn2cc(Nc3ccc(CNc4ncc(CC(=O)NC5COC5)cn4)cc3)nc12] | ¹H NMR (400 MHz, DMSO-d₆) δ (ppm) 8.84 (s, 1H), 8.80 (d, 1H), 8.13 (s, 2H), 8.06 (d, 1H), 7.62 (s, 1H), 7.48 (t, 1H), 7.35 (d, J = 4.52 Hz, 1H), 7.27 (d, 2H), 7.18 (d, 2H), 4.70-4.80 (m, 1H), 4.64-4.70 (m, 2H), 4.32-4.47 (m, 4H), 4.00 (s, 3H), 3.24 (s, 2H); (ESI) m/z. [M + H]+ 461.1 |
| | PA253 | |
| PA254 | 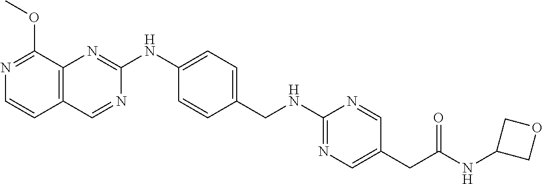 [SMILES: COc1nccc2cnc(Nc3ccc(CNc4ncc(CC(=O)NC5COC5)cn4)cc3)nc12] | ¹H NMR (400 MHz, DMSO-d6) δ (ppm) 10.05 (s, 1H), 9.28 (s, 1H), 8.74 (d, 1H), 8.11 (s, 2H), 7.88 (d, 1H), 7.84 (d, 2H), 7.51 (t, 1H), 7.31 (d, 1H), 7.23 (d, 2H), 4.72 (dt, 1H),4.65 (t, 2H), 4.41 (d, 2H), 4.37 (t, 2H), 4.01 (s, 3H), 3.22 (s, 2H). (ESI) m/z. [M + H]⁺ 473.0 |
| | PA254 | |
| PA255 | 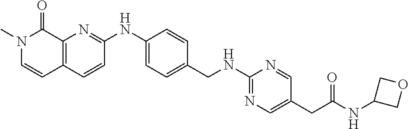 [SMILES: Cn1ccc2ccc(Nc3ccc(CNc4ncc(CC(=O)NC5COC5)cn4)cc3)nc2c1=O] | ¹H NMR (400 MHz, DMSO-d6) δ (ppm) 9.41 (s, 1H), 8.77 (s, 1H), 8.14 (d, 2H), 7.88 (dd, 3H), 7.52 (s, 1H), 7.34 (d, 1H), 7.18 (dd, 3H), 6.45 (d, 1H), 4.72 (dt, 3H), 4.41 (dd, 4H), 3.51 (d, 3H), 3.25 (s, 2H); (ESI) m/z. [M + H]+ 472.0 |
| | PA255 | |
| PA256 | 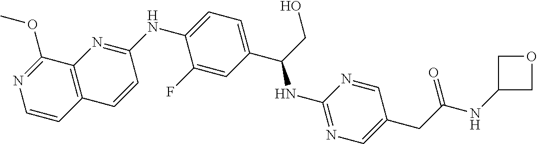 [SMILES: COc1nccc2ccc(Nc3ccc([C@@H](CO)Nc4ncc(CC(=O)NC5COC5)cn4)cc3F)nc12] | ¹H NMR (400 MHz, DMSO-d6) δ (ppm) 9.19 (s, 1H), 8.74 (d, 1H), 8.61 (t, 1H), 8.09 (s, 2H), 8.01 (d, 1H), 7.82 (s, 1H), 7.41 (dd, 1H), 7.33 (d, 1H), 7.25-7.14 (m, 3H), 5.00-4.82 (m,2H), 4.70 (dt, 1H), 4.63 (d, 2H), 4.38-4.33 (m, 2H),3.97 (s, 3H), 3.62 (q, 2H), 3.20 (s, 2H); (ESI) m/z. [M + H]+ 520.3 |
| | PA256 | |
| PA257 | 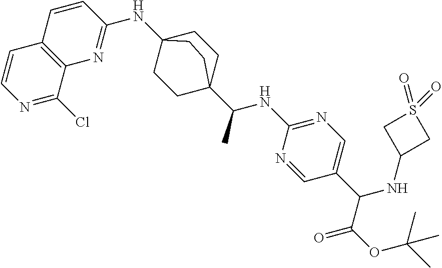 [SMILES: C[C@H](Nc1ncc(C(NC2CS(=O)(=O)C2)C(=O)OC(C)(C)C)cn1)C12CCC(Nc3ccc4ccnc(Cl)c4n3)(CC1)CC2] | ¹H NMR (400 MHz, DMSO-d₆) δ (ppm) 8.19 (s, 2H), 7.95 (d, 1H), 7.87 (d, 1H), 7.53 (d, 1H), 7.18 (s, 1H), 7.01 (d, 1H), 6.95 (d, 1H), 4.36-4.16 (m, 1H), 4.09 (d, 1H), 4.01-3.96 (m, 2H), 3.95 (s, 1H), 3.48-3.42 (m, 2H), 2.08 (t, 6H), 1.67-1.50 (m, 6H), 1.38 (s, 9H), 1.02 (d, 3H); (ESI) m/z. [M + H]+ 642.4 |
| | PA257 | |
| PA258 | 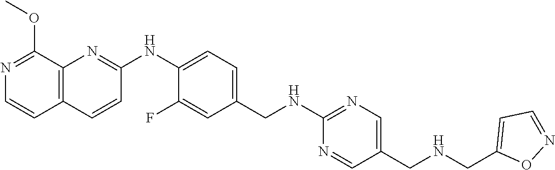 [SMILES: COc1nccc2ccc(Nc3ccc(CNc4ncc(CNCc5ccno5)cn4)cc3F)nc12] | ¹H NMR (400 MHz, DMSO-d₆) δ (ppm) 9.19 (s, 1H), 8.63 (dt, 1H), 8.44 (d, 1H), 8.21 (s, 2H), 8.01 (d, 1H), 7.82 (d, 1H), 7.64 (t, 1H), 7.41 (dd, 1H), 7.20 (d, 1H), 7.17-7.09 (m, 2H), 6.37 (s, 1H), 4.45 (d, 2H), 3.96 (s, 3H), 3.83 (s, 2H), 3.52 (s, 2H); (ESI) m/z. [M + H]+ 487.1 |
| | PA258 | |
| PA259 | 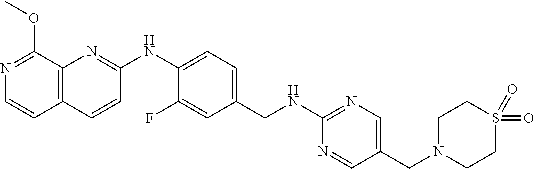 [SMILES: COc1nccc2ccc(Nc3ccc(CNc4ncc(CN5CCS(=O)(=O)CC5)cn4)cc3F)nc12] | ¹H NMR (400 MHz, DMSO-d6) d 9.22 (s, 1H), 8.66 (t, 1H), 8.25 (s, 2H), 8.05 (d, 1H), 7.86 (d, 1H), 7.72 (br t, 1H), 7.44 (d, 1H), 7.28 − 7.12 (m, 3H), 4.50 (br d, 2H), 4.01 (s, 3H), 3.50 (s, 2H), 3.09 (br s, 4H), 2.86 (br s, 4H); (ESI) m/z. [M + H]+ 524.1 |
| | PA259 | |
| PA260 | 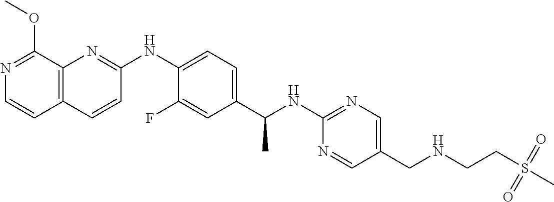 [SMILES: COc1nccc2ccc(Nc3ccc([C@H](C)Nc4ncc(CNCCS(C)(=O)=O)cn4)cc3F)nc12] | ¹H NMR (400 MHz, DMSO-d6) δ (ppm) 9.21-9.17 (m, 1H), 8.61 (t, 1H), 8.17 (s, 2H), 8.01 (d, 1H), 7.82 (d, 1H), 7.57 (d, 1H), 7.40 (d, 1H), 7.26-7.15 (m, 3H), 5.05 (p, 1H), 3.97 (s, 3H), 3.44 (s, 2H), 3.17 (t, 2H), 2.96 (s, 3H), 2.81 (t, 2H), 1.41 (d, 3H); (ESI) m/z. [M + H]+ 526.0 |
| | PA260 | |
| PA261 | 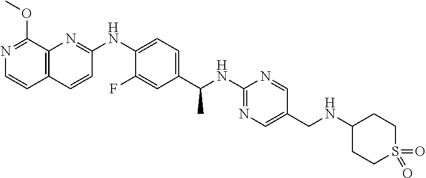 [SMILES: COc1nccc2ccc(Nc3ccc([C@H](C)Nc4ncc(CNC5CCS(=O)(=O)CC5)cn4)cc3F)nc12] | ¹H NMR (400 MHz, DMSO-d6) δ (ppm) 9.23 (d, 1H), 8.64 (t, 1H), 8.25 (s, 2H), 8.05 (d, 1H), 7.85 (d, 1H), 7.70 (d, 1H), 7.43 (d, 1H), 7.29-7.19 (m, 3H), 5.07 (q, 1H), 4.01 (s, 3H), 3.58 (s, 2H), 3.17-3.02 (m, 4H), 2.87 (s, 1H), 2.17-2.06 (m, 2H), 1.95-1.84 (m, 2H), 1.45 (d, 3H); (ESI) m/z. [M + H]+ 552.0 |
| | PA261 | |
| PA262 | 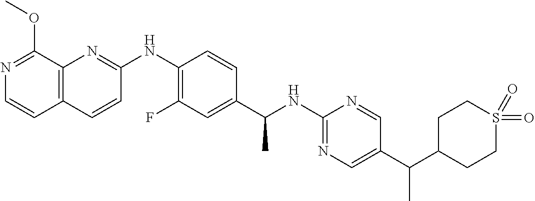 [SMILES: COc1nccc2ccc(Nc3ccc([C@H](C)Nc4ncc(C(C)C5CCS(=O)(=O)CC5)cn4)cc3F)nc12] | ¹H NMR (400 MHz, DMSO-d6) δ (ppm) 9.23 (s, 1H), 8.65 (t, 1H), 8.14 (s, 2H), 8.05 (d, 1H), 7.85 (d, 1H), 7.58 (d, 1H), 7.44 (d, 1H), 7.30-7.18 (m, 3H), 5.10-5.03 (m, 1H), 4.01 (s, 3H), 3.13-2.90 (m, 4H), 2.56-2.51 (m, 1H), 2.03 (d, 1H), 1.85-1.65 (m, 2H), 1.61-1.46 (m, 2H), 1.44 (d, 3H), 1.16 (d, 3H); (ESI) m/z. [M + H]+ 551.0 |
| | PA262 | |
| PA263 | 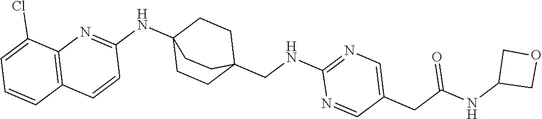 [SMILES: O=C(Cc1cnc(NCC23CCC(Nc4ccc5cccc(Cl)c5n4)(CC2)CC3)nc1)NC1COC1] | ¹H NMR (400 MHz, DMSO-d6) δ (ppm) 8.79 (d, 1H), 8.10 (s, 2H), 7.80 (d, 1H), 7.60 (dd, 1H), 7.53 (d, 1H), 7.06 (t, 1H), 6.94 (t, 1H), 6.82(s, 1H), 6.79 (d, 1H), 4.73-4.81 (m, 1H), 4.69 (t, 2H), 4.41 (t, 2H), 3.24 (s, 2H), 3.12 (d, 2H), 2.03-2.16 (m, 6H), 1.47-1.61 (m, 6H); (ESI) m/z. [M + H]+ 507.1 |
| | PA263 | |
| PA264 | 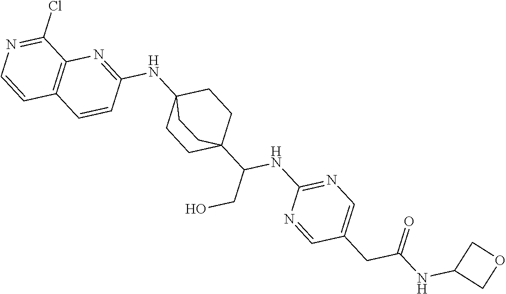 [SMILES: O=C(Cc1cnc(NC(CO)C23CCC(Nc4ccc5ccnc(Cl)c5n4)(CC2)CC3)nc1)NC1COC1] | ¹H NMR (400 MHz, DMSO-d6) δ (ppm) 8.77 (d, 1H), 8.05 (s, 2H), 7.91 (d, 1H), 7.82 (d, 1H), 7.49 (d, 1H), 7.12 (s, 1H), 6.97 (d, 1H), 6.52 (d, 1H), 4.77-4.69 (m, 1H), 4.66 (t, 2H), 4.36 (dt, 3H), 3.87 (td, 1H), 3.58 (dt, 1H), 3.37 (d, 1H), 3.20 (s, 2H), 2.02 (t, 6H), 1.71-1.46 (m, 6H); (ESI) m/z. [M + H]+ 538.4 |
| | PA264 | |
| PA265 | 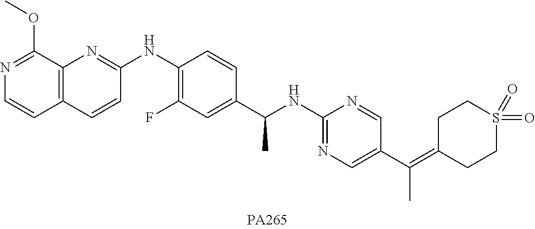 | ¹H NMR (400 MHz, DMSO-d₆) δ (ppm) 9.21 (s, 1H), 8.65-8.56 (m, 1H), 8.02 (d, 1H), 7.82 (d, 1H), 7.77 (s, 2H), 7.40 (dd, 1H), 7.25-7.19 (m, 2H), 7.16 (dd, 1H), 4.94 (q, 1H), 3.97 (d, 5H), 3.85 (td, 2H), 3.78-3.70 (m, 1H), 1.40 (d, 3H); (ESI) m/z. [M + H]+ 471.3 |
| | PA265 | |
| PA266 | 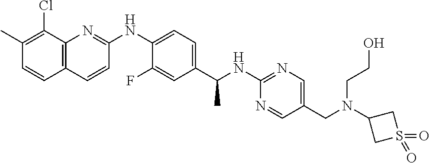 [SMILES: Cc1ccc2ccc(Nc3ccc([C@H](C)Nc4ncc(CN(CCO)C5CS(=O)(=O)C5)cn4)cc3F)nc2c1Cl] | ¹H NMR (400 MHz, CDCl₃) δ (ppm) 9.16 (t, 1H), 8.16 (s, 2H), 7.93 (d, 1H), 7.50 (d, 1H), 7.28-7.32 (m, 1H), 7.23 (d, 1H), 7.17 (dd, 1H), 7.02 (br s, 1H), 6.88 (d, 1H), 5.61-5.68 (m, 1H), 5.16 (m, 1H), 4.05-4.24 (m, 4H), 3.70-3.81 (m, 1H), 3.65 (t, 2H), 3.55 (s, 2H), 2.72 (t, 2H), 2.63 (s, 3H), 1.60 (d, 3H); (ESI) m/z. [M + H]+ 585.3 |
| | PA266 | |
| PA267 | 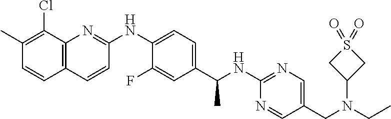 [SMILES: CCN(Cc1cnc(N[C@@H](C)c2ccc(Nc3ccc4ccc(C)c(Cl)c4n3)c(F)c2)nc1)C1CS(=O)(=O)C1] | ¹H NMR (400 MHz, CDCl₃) δ (ppm) 9.05 (t, 1H), 8.10 (s, 2H), 8.06 (s, 0.4H, HCOOH), 7.84 (d, 1H), 7.40 (d, 1H), 7.19-7.23 (m, 1H), 7.13 (d, 1H), 7.09 (dd, 1H), 6.79 (d, 1H), 5.86-5.98 (m, 1H), 5.04-5.11 (m, 1H), 3.92-4.10 (m, 4H), 3.53-3.61 (m, 1H), 3.36 (s, 2H), 2.54 (s, 3H), 2.48 (q, 2H), 1.51 (d, 3H), 0.95 (t, 3H); (ESI) m/z. [M + H]+ 569.3 |
| | PA267 | |
| PA268 | 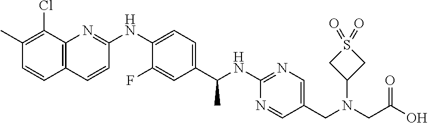 [SMILES: Cc1ccc2ccc(Nc3ccc([C@H](C)Nc4ncc(CN(CC(=O)O)C5CS(=O)(=O)C5)cn4)cc3F)nc2c1Cl] PA268 | ¹H NMR (400 MHz, DMSO-d₆) δ (ppm) 9.22 (s, 1H), 9.11 (t, 1H), 8.19 (s, 2H), 8.08 (d, 1H), 7.69 (d, 1H), 7.63 (d, 1H), 7.33 (d, 1H), 7.18-7.30 (m, 3H), 5.05-5.14 (m, 1H), 4.12-4.31 (m, 4H), 3.85-3.94 (m, 1H), 3.52 (s, 2H), 3.19 (s, 2H), 2.53 (s, 3H), 1.45 (d, 3H); (ESI) m/z. [M + H]+ 599.3 |
| PA269 | 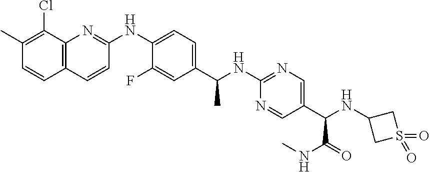 [SMILES: CNC(=O)[C@H](NC1CS(=O)(=O)C1)c1cnc(N[C@@H](C)c2ccc(Nc3ccc4ccc(C)c(Cl)c4n3)c(F)c2)nc1] | (ESI) m/z. [M + H]+ 598.2 |
| | PA269 | |
| PA270 | 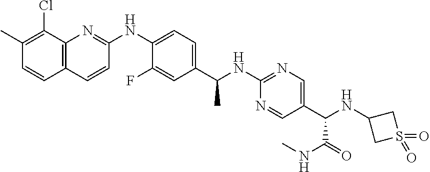 [SMILES: CNC(=O)[C@@H](NC1CS(=O)(=O)C1)c1cnc(N[C@@H](C)c2ccc(Nc3ccc4ccc(C)c(Cl)c4n3)c(F)c2)nc1] | (ESI) m/z. [M + H]+ 598.2 |
| | PA270 | |
| PA271 | 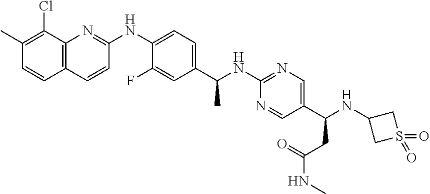 [SMILES: CNC(=O)C[C@H](NC1CS(=O)(=O)C1)c1cnc(N[C@@H](C)c2ccc(Nc3ccc4ccc(C)c(Cl)c4n3)c(F)c2)nc1] | (ESI) m/z. [M + H]+ 612.2 |
| | PA271 | |
| PA272 | 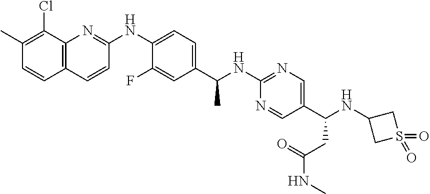 [SMILES: CNC(=O)C[C@@H](NC1CS(=O)(=O)C1)c1cnc(N[C@@H](C)c2ccc(Nc3ccc4ccc(C)c(Cl)c4n3)c(F)c2)nc1] | (ESI) m/z. [M + H]+ 612.2 |
| | PA272 | |
| PA273 | 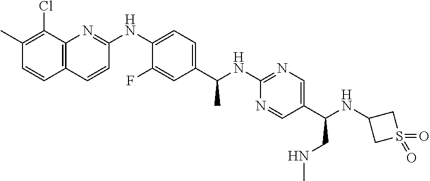 [SMILES: CNC[C@H](NC1CS(=O)(=O)C1)c1cnc(N[C@@H](C)c2ccc(Nc3ccc4ccc(C)c(Cl)c4n3)c(F)c2)nc1] | (ESI) m/z. [M + H]+ 584.2 |
| | PA273 | |
| PA274 | 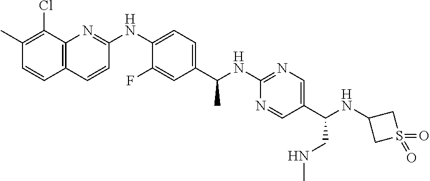 [SMILES: CNC[C@@H](NC1CS(=O)(=O)C1)c1cnc(N[C@@H](C)c2ccc(Nc3ccc4ccc(C)c(Cl)c4n3)c(F)c2)nc1] | (ESI) m/z. [M + H]+ 584.2 |
| | PA274 | |
| PA275 | 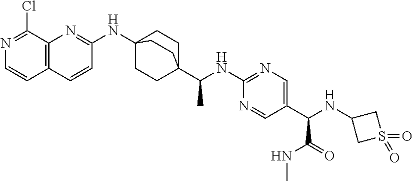 [SMILES: CNC(=O)[C@H](NC1CS(=O)(=O)C1)c1cnc(N[C@@H](C)C23CCC(Nc4ccc5ccnc(Cl)c5n4)(CC2)CC3)nc1] | (ESI) m/z. [M + H]+ 599.2 |
| | PA275 | |
| PA276 | 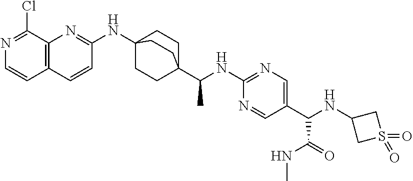 | (ESI) m/z. [M + H]+ 599.2 |
| | PA276 | |
| PA277 | 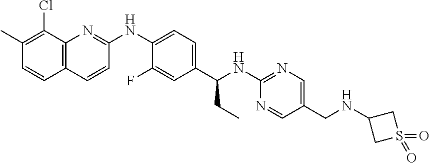 [SMILES: CC[C@H](Nc1ncc(CNC2CS(=O)(=O)C2)cn1)c1ccc(Nc2ccc3ccc(C)c(Cl)c3n2)c(F)c1] | ¹H NMR (400 MHz, DMSO-d6) δ (ppm) 9.22 (d, 1H), 9.12 (t, 1H), 8.18 (s, 2H), 8.07 (d, 1H), 7.54-7.70 (m, 2H), 7.33 (d, 1H), 7.24-7.30 (m, 2H), 7.22 (br d, 1H), 4.79-4.92 (m, 1H), 4.19-4.35 (m, 2H), 3.83-3.92 (m, 2H), 3.41-3.51 (m, 1H), 3.38 (s, 2H), 2.80 (br s, 1H), 2.53 (s, 3H), 1.69-1.87 (m, 2H), 0.90 (t, 3H) (ESI) m/z. [M + H]+ 555.0 |
| | PA277 | |
| PA278 | 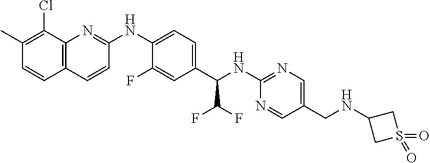 [SMILES: Cc1ccc2ccc(Nc3ccc([C@@H](Nc4ncc(CNC5CS(=O)(=O)C5)cn4)C(F)F)cc3F)nc2c1Cl] | (ESI) m/z. [M + H]+ 577.1 |
| | PA278 | |
| PA279 | 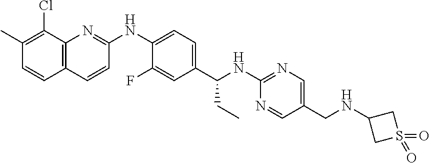 [SMILES: CC[C@@H](Nc1ncc(CNC2CS(=O)(=O)C2)cn1)c1ccc(Nc2ccc3ccc(C)c(Cl)c3n2)c(F)c1] | ¹H NMR (400 MHz, DMSO-d6) δ (ppm) 9.22 (d, 1H), 9.12 (t, 1H), 8.18 (s, 2H), 8.07 (d, 1H), 7.53-7.69 (m, 2H), 7.33 (d, 1H), 7.24-7.30 (m, 2H), 7.22 (br d, 1H), 4.77-4.92 (m, 1H), 4.20-4.34 (m, 2H), 3.83-3.92 (m 2H), 3.41-3.50 (m, 1H), 3.38 (s, 2H), 2.80 (br s, 1H), 2.53 (s, 3H), 1.69-1.87 (m, 2H), 0.90 (t, 3H) (ESI) m/z. [M + H]+ 555.0 |
| | PA279 | |

### Biological Test Example

### G401 ELISA Test:

### Main Experimental Instruments and Equipment

Plate Reader: Envision (Perkinelmner); plate shaker: IKA MTS 2/4; plate Washer: BioTek microplate wisher 405 select; Cell counter: Counterstar (Ruiyu-biotech); Cell culture incubator: MCO-15AC (ThermoFisher); Pipette: BioHit Multichannel. Pipette: 0.2-10 μL, 10-300 μL, 50-1200 μL; Centrifuge: Thermo Centrifuge ST 40R; Pure water system: Millipore Milli-Q Reference system; Refrigerator: Haier −80 degree, −20 degree, and 4-degree refrigerators; cell cryopreservation box: Mr. Frosty™ Gradient Cooling Box Cat #5100-0001

### Experimental Cell Culture Reagents, Consumables, and their Sources

McCoys 5A (Modified) culture medium (ATCC); FBS (Gibco); 0.25% Trypsin/EDTA (Gibco); PBS (Hyclone); Penicillin/Streptomycin(100×) (Gibco); DMSO (Sigma); 384 cell culture plate (Corning); 384 detection plate (Greiner); 96 well plate (compound dilution) (Haimen, Qunchao); T75 Cell Culture Bottle (Corning)

### Experimental Operation

G401 cells were seeded in a 384-well cell culture plate and incubated overnight at 37° C. in a 5% CO₂ cell culture incubator, after which the cells were treated with compounds of different concentrations for 72 hours. The 384-well cell culture plate was taken out and upside down in a centrifuge to centrifugeat 300 rpm for 1 minute. After centrifugation, the 384-well cell culture plate was washed twice with PBS, added with lysis solution and lysed for 60 minutes. Then neutralization buffer was added for mixing and incubation was continued for 30 minutes. 20 ul (for detecting K27) and 10 ul (for detecting H3) of cell lysate were extracted from the 384-well cell culture plate and transferred to a 384 ELISA detection plate and incubate overnight at 4° C. The next day, the 384 ELISA plate was taken out and washed 5 times with TBST in a microplate washer. Then, the plate was added with a blocking solution and sealed at room temperature for 1 hour. The blocking solution was discarded and the primary antibody was added and then the plate was incubated at room temperature for 1 hour. After incubation, the plate was washed with TBST 5 times in a microplate washer, then added with secondary antibody and incubated at room temperature for 1 hour. After incubation, the plate was washed with TBST 5 times in a microplate washer, and finally added with color developer for color development. The plate was read with Envision plate reader. The obtained data was converted into inhibition rate using the following calculation method: % Inhibition rate=100−100*(compound absorbance−positive control absorbance)/(negative control absorbance−positive control absorbance), and then the inhibition rate data was subjected to curve fitting using the “logarithmic (inhibitor) and reaction-variable slope (four parameters)” model in GraphPad Prism 5 software.

The inhibition rate of the compound was calculated as follows:

$$\% Inhibition rate = 100 - 100 * ( compound absorance - positive control absorbance ) / ( negative control absorbance - positive control absorbance )$$

The compounds of the present invention were tested according to the above experimental method, and some results were shown in Table 3. The test results were classified by A, B, and C, wherein A represents IC₅₀≤0.1 uM; B represents 0.1 uM<IC₅₀≤1 uM; C represents IC₅₀>1 uM

TABLE 3

| | G401_ELISA | | G401_ELISA | | G401_ELISA |
|---|---|---|---|---|---|
| Number | IC₅₀ | Number | IC₅₀ | Number | IC₅₀ |
| PA001 | A | PA044 | B | PA073 | C |
| PA007 | C | PA046 | A | PA074 | C |
| PA010 | B | PA041 | A | PA076 | A |
| PA011 | B | PA048 | C | PA079 | A |
| PA018 | A | PA051 | C | PA081 | B |
| PA024 | B | PA066 | C | PA082 | C |
| PA025 | A | PA067 | C | PA151 | C |
| PA034 | A | PA068 | B | PA152 | B |
| PA038 | C | PA069 | C | PA153 | A |
| PA039 | B | PA070 | C | PA154 | B |
| PA042, | A, B | PA054, | B, C | PA061, | A, B |
| PA043 | | PA055 | | PA062 | |
| PA077, | A, B | PA162 | A | PA163 | A |
| PA078 | | | | | |
| PA164 | A | PA165 | A | PA166, | A, A |
| | | | | PA207 | |
| PA167 | A | PA168 | A | PA169 | A |
| PA170 | A | PA171 | A | PA172 | A |
| PA173 | A | PA174 | A | PA175 | A |
| PA176, | A, B | PA177 | A | PA178 | A |
| PA209 | | | | | |
| PA179 | A | PA180 | A | PA181 | A |
| PA182 | A | PA183 | A | PA184 | A |
| PA185 | A | PA186, | A, B | PA187 | A |
| | | PA225 | | | |
| PA188 | A | PA189 | A | PA190 | A |
| PA191 | A | PA192 | A | PA193 | A |
| PA194 | A | PA195 | A | PA196 | A |
| PA197 | A | PA198 | A | PA199 | A |
| PA200 | A | PA201 | A | PA202 | A |
| PA203 | A | PA204 | A | PA205 | A |
| PA206 | A | PA208 | B | PA210 | B |
| PA211 | B | PA212 | B | PA213 | B |
| PA214 | B | PA215 | B | PA216 | B |
| PA217 | B | PA218 | B | PA219 | B |
| PA220 | B | PA221 | B | PA222 | B |
| PA223 | B | PA224 | B | PA226 | B |
| PA227 | B | PA228 | B | PA229 | B |
| PA230 | B | PA231 | B | PA232 | B |
| PA233 | B | PA234 | B | PA235 | B |
| PA236 | B | PA237 | B | PA238 | B |
| PA239 | B | PA240 | B | PA241 | B |
| PA242 | C | PA243 | C | PA244 | C |
| PA245 | C | PA246 | C | PA247 | C |
| PA248 | C | PA249 | C | PA250 | C |
| PA251 | C | PA252 | C | PA253 | C |
| PA254 | C | PA255 | C | PA256 | C |
| PA257 | A | PA258 | A | PA259 | A |
| PA260 | A | PA261 | A | PA262 | A |
| PA263 | A | PA264 | A | PA265 | A |
| PA266 | A | PA267 | A | PA268 | A |

All documents mentioned in the present invention are cited as references in this application, just as each document is individually cited as a reference. In addition, it should be understood that, after reading the above teaching content of the present invention, those skilled in the art can make various changes or modifications to the present invention, and these equivalent forms also fall within the scope defined by the appended claims of the present application.

1. A compound of formula I, or a pharmaceutically acceptable salt thereof, or a deuterated compound, a racemic mixture, or an optical monomer thereof: 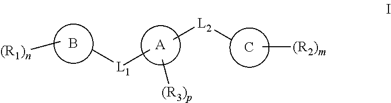
wherein, ring A is selected from the group consisting of: 5-10 membered bridged ring (including carbocycle and heterocycle), 6-10 membered aromatic ring, and 5-10 membered heteroaromatic ring;
ring B is selected from the group consisting of: 6-10 membered aromatic ring, and 5-14 membered heteroaromatic ring;
ring C is selected from the group consisting of: 5-10 membered aromatic ring or partially saturated aromatic ring, 5-10 membered heteroaromatic ring or partially saturated heteroaromatic ring, 5-10 membered saturated or partially unsaturated carbocycle (including fused and bridged rings), 5-10 membered saturated or partially unsaturated heterocycle (including fused and bridged rings);
L₁ and L₂ are each independently -(L)ₚ-, and each L is independently selected from the group consisting of: chemical bond, none, —O—, —CHR—, —CHR—NH—, carbonyl, S, —NR—, —NHC(O)—, —NHS(O)₂—, —NHC(O)NH—, —NHC(S)NH—, —COO—, —O—S(O)₂—, —CHR—NR—, —C(R)₂NR—, and —C(R)₂—; wherein, each R is independently selected from the group consisting of: H, substituted or unsubstituted C₁₋₄ alkyl, substituted or unsubstituted C₃₋₄ cycloalkyl, and substituted or unsubstituted 3-4 membered heterocyclyl;
n is selected from the group consisting of: 0, 1, 2, 3, 4, and 5;
m is selected from the group consisting of: 0, 1, 2, 3, and 4;
p is selected from the group consisting of: 0, 1, and 2;
each R₁, R₂, and R₃ is independently selected from the group consisting of: H, halogen, cyano, amino, nitro, hydroxyl, thiol, aldehyde group, carboxyl, sulfonyl, substituted or unsubstituted C₁-C₆ alkyl, substituted or unsubstituted C₁-C₆ alkoxy, substituted or unsubstituted C₁-C₆alkylamino, substituted or unsubstituted C₁-C₆ alkylamide, substituted or unsubstituted C₁-C₆ alkyl-C(O)O, or substituted or unsubstituted C₁-C₆ alkyl-OC(O), substituted or unsubstituted amide, substituted or unsubstituted amino (NH₂), substituted or unsubstituted C₂-C₆ alkenyl, substituted or unsubstituted C₂-C₆ alkynyl, substituted or unsubstituted C₃-C₆ carbocycle (including saturated or partially unsaturated situation), substituted or unsubstituted 3-6 membered heterocycle (including saturated or partially unsaturated situation), and —X—Z—Y—R₅;
wherein, X and Y are each independently selected from the group consisting of: chemical bond, —O—, —C(R₅)₂—, —S—, and —NR₅—;
Z is selected from the group consisting of: C(O), NH, CH═CH, —C(R₅)₂—, S(O), and S(O)₂;
each R₅ is independently selected from the group consisting of: H, halogen, cyano, amino, nitro, hydroxyl, thiol, aldehyde group, carboxyl, sulfonyl, substituted or unsubstituted C₁-C₆ alkyl, substituted or unsubstituted C₃-C₆ carbocycle (including saturated or partially unsaturated situation), substituted or unsubstituted 3-6 membered heterocycle, substituted or unsubstituted C₆₋₁₀ aromatic ring, substituted or unsubstituted 5 to 12 membered heteroaromatic ring, substituted or unsubstituted C₁-C₆ alkyl-C(O)O, or substituted or unsubstituted C₁-C₆ alkyl-OC(O), and substituted or unsubstituted 5 to 9 membered heterospiro ring;
or two R₁, R₂, or R₃ located at two adjacent ring atoms together form a fused ring structure selected from the group consisting of: substituted or unsubstituted C₆₋₁₀ aromatic ring, substituted or unsubstituted 5 to 12 membered heteroaromatic ring, substituted or unsubstituted C₃-C₈ carbocycle (including saturated or partially unsaturated situation), substituted or unsubstituted 3 to 8 membered heterocycle (including saturated or partially unsaturated situation); or two adjacent R₁, R₂, or R₃ located at the same ring carbon atom together with the attached ring form a 3 to 8 membered saturated or partially unsaturated spiro ring structure, or a 3 to 8 membered saturated or partially unsaturated heterospiro ring structure (the 3-8 membered ring referred herein is the ring formed by the substituents, which does not include the attached ring);
wherein, the ring skeleton of each heterocycle mentioned above may contain 1-3 heteroatoms selected from boron, oxygen, sulfur, phosphorus, and nitrogen; specifically, when the atom is boron, sulfur, phosphorus, or nitrogen, the ring skeleton atom can be oxidized, such as S(O) or S(O)₂;
unless otherwise specified, the “substituted” refers to the corresponding group is substituted by one or more substituents selected from the group consisting of: deuterium, tritium, halogen, oxo, =NH, =N(C₁₋₈ alkyl), hydroxy, carboxy, thiol, benzyl, C₁-C₁₂ alkoxycarbonyl, C₁-C₆ aldehyde group, amino, C₁-C₆ amide, nitro, cyano, unsubstituted or halogenated C₁-C₆ alkyl, C₁-C₆ alkyl-CN, unsubstituted or halogenated C₃-C₈ cycloalkyl, C₂-C₁₀ alkenyl, C₂-C₁₀ alkynyl, C₁-C₆ alkoxy, C₁-C₆ alkyl-amino, C₆-C₁₀ aryl, 5- or 6-membered heteroaryl, 3-8 membered non-aromatic heterocyclyl, —O—(C₆-C₁₀ aryl), —O-(5- or 6-membered heteroaryl), C₁-C₁₂ alkylamino carbonyl, unsubstituted or halogenated C₂-C₁₀ acyl, sulfonyl (—SO₂—OH), phosphoryl (—PO₃—OH), unsubstituted or halogenated C₁-C₄ alkyl-S(O)₂—, unsubstituted or halogenated C₁-C₄ alkyl-SO—, and unsubstituted or halogenated C₁-C₄ alkylamino-S(O)₂—;
wherein, each chiral atom in the molecule can be in R configuration, S configuration, or a combination thereof.

2. The compound according to claim 1, or a pharmaceutically acceptable salt thereof or adeuterated compound thereof, characterized in that ring B is a 6 to 10 membered heteroaromatic ring, and R₁ is selected from the group consisting of: H, halogen, cyano, amino, nitro, hydroxyl, thiol, aldehyde group, carboxyl, sulfonyl, substituted or unsubstituted C₁-C₆ alkyl, substituted or unsubstituted C₁-C₆ alkoxy, and substituted or unsubstituted C₁-C₆ alkylamino; or two R₁ connected to adjacent ring atoms together form a cyclic structure selected from the group consisting of: substituted or unsubstituted C₃-C₆ carbocycle (including saturated or partially unsaturated situation), substituted or unsubstituted 3 to 6 membered heterocycle (including saturated and partially unsaturated situation).

3. The compound according to claim 1, or a pharmaceutically acceptable salt thereof or adeuterated compound thereof, characterized in that ring B has a structure selected from the group consisting of (wherein, the connection site can be on any ring atom):

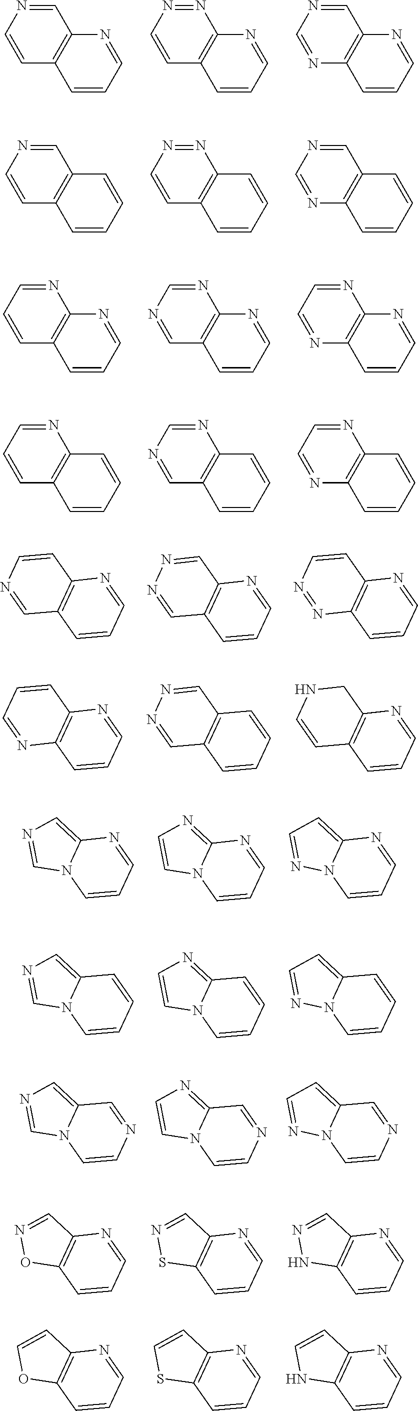

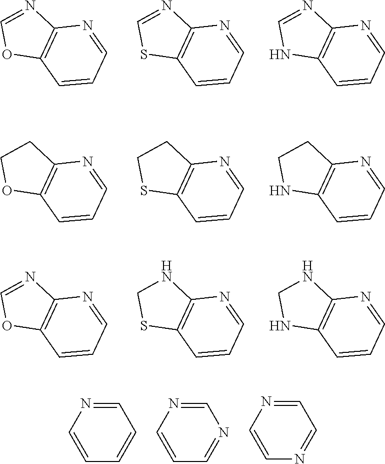

4. The compound according to claim 1, or a pharmaceutically acceptable salt thereof or adeuterated compound thereof, characterized in that ring A has a structure selected from the group consisting of (wherein, the connection site can be on any ring atom):

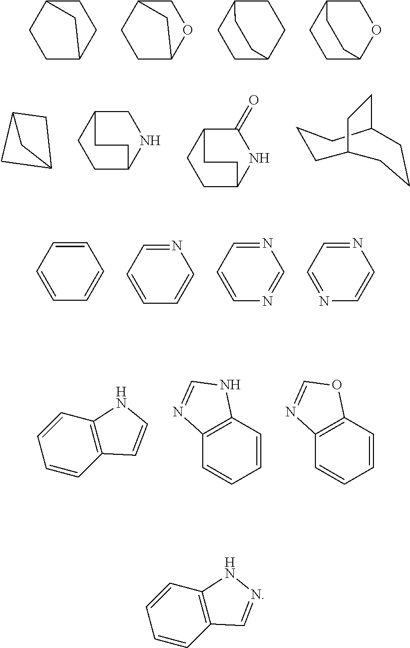

5. The compound according to claim 1, or a pharmaceutically acceptable salt thereof or adeuterated compound thereof, characterized in that R₂ has a structure as shown in the following formula: 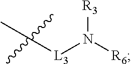
wherein, L₃ is selected from the group consisting of: —C(R₅)₂—C(O)— and —C(R₅)₂—;
R₃ is selected from the group consisting of: H, substituted or unsubstituted C₁-C₆ alkyl;
R₆ is selected from the group consisting of: substituted or unsubstituted C₁-C₆ alkyl, substituted or unsubstituted C₃-C₆carbocycle (including saturated or partially unsaturated situation), substituted or unsubstituted 3-6 membered heterocycle; the ring skeleton of the heterocycle may contain 1-3 heteroatoms selected from oxygen and sulfur; and the ring skeleton atoms can be oxidized;
the “substituted” refers to the hydrogen atoms on the corresponding group is substituted by one or more substituents selected from the group consisting of: deuterium, tritium, halogen, oxo, hydroxyl, carboxyl, thiol, benzyl, cyano, unsubstituted or halogenated C₁-C₆ alkyl, unsubstituted or halogenated C₃-C₈ cycloalkyl, C₂-C₁₀ alkenyl, C₁-C₆ alkoxy, C₁-C₆ alkyl-amino, C₆-C₁₀ aryl, 5- or 6-membered heteroaryl, and 3-8 membered non-aromatic heterocyclyl.

6. The compound according to claim 1, or a pharmaceutically acceptable salt thereof or adeuterated compound thereof, characterized in that R₂ is —CHR—C(O)NH—R₆, wherein R is H or substituted or unsubstituted C₁₋₄ alkyl, substituted or unsubstituted C₁-C₆ alkoxy, hydroxyl, substituted or unsubstituted amino; R₆ is selected from the group consisting of: substituted or unsubstituted C₃-C₆ carbocycle (including saturated or partially unsaturated situation), and substituted or unsubstituted 3-6 membered heterocycle (including saturated or partially unsaturated situation).

7. The compound according to claim 1, or a pharmaceutically acceptable salt thereof or adeuterated compound thereof, characterized in that the compound has a structure as shown in formula II, III, or IV: 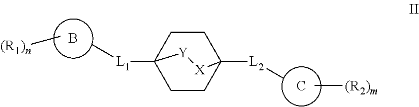
wherein, X and Y are independently selected from the group consisting of: O, NR₃, C(R₃)₂ and —C(═O)—;
or the compound has a structure as shown in formula III below: 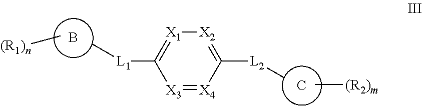
wherein, X₁, X₂, X₃, or X₄ is each independently selected from the group consisting of: N, and CR₃;
or the compound has a structure as shown in Formula IV below: 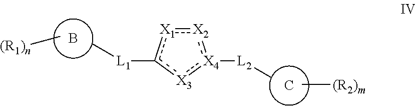
wherein, X₁, X₂, X₃, or X₄ is each independently selected from the group consisting of: O, S, N, NR₃ and CR₃; [symbol]
represents single bond or double bond;
the remaining groups are as defined in claim 1.

8. The compound according to claim 1, or a pharmaceutically acceptable salt thereof or a deuterated compound thereof, characterized in that L₁ is selected from the group consisting of: chemical bond, —O—, —CHR—, carbonyl, S and —NH—; L₂ is selected from the group consisting of: chemical bond, —CHR—NH—, —CHR—O—, —CHR—S—, and —(CHR)₂—.

9. The compound according to claim 1, or a pharmaceutically acceptable salt thereof or adeuterated compound thereof, characterized in that the compound is selected from the group consisting of:

| Number | Structure | Number | Structure |
|---|---|---|---|
| PA001 | 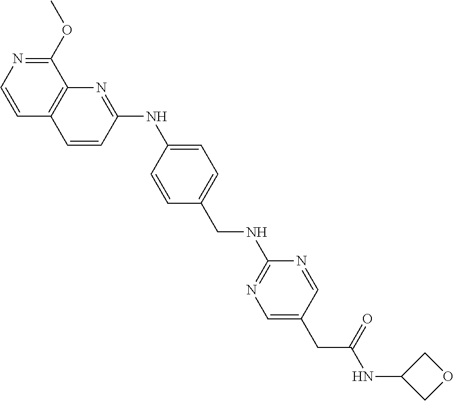 [SMILES: COc1nccc2ccc(Nc3ccc(CNc4ncc(CC(=O)NC5COC5)cn4)cc3)nc12] | PA185 | 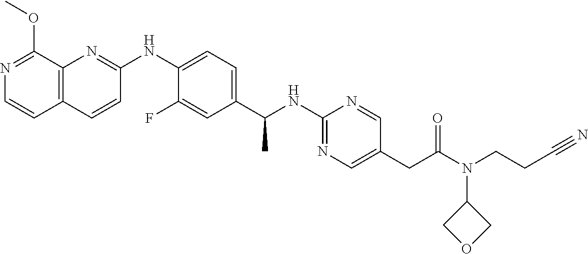 PA185 |
| | PA001 | | |
| PA007 | 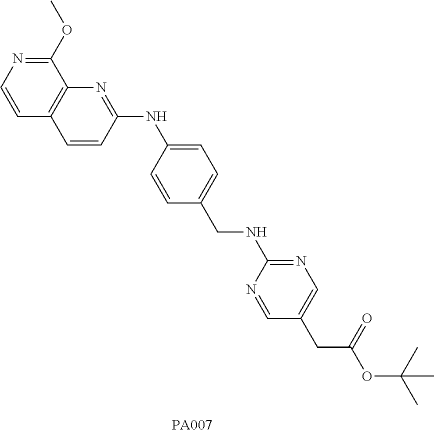 [SMILES: COc1nccc2ccc(Nc3ccc(CNc4ncc(CC(=O)OC(C)(C)C)cn4)cc3)nc12] | PA186 | |
| PA010 | 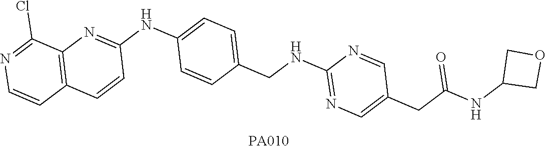 [SMILES: O=C(Cc1cnc(NCc2ccc(Nc3ccc4ccnc(Cl)c4n3)cc2)nc1)NC1COC1] | PA187 | 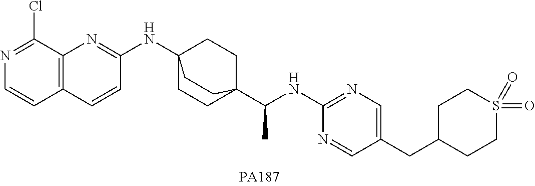 |
| PA033 | 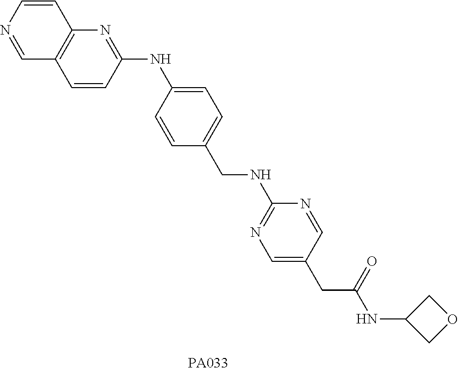 [SMILES: O=C(Cc1cnc(NCc2ccc(Nc3ccc4cnccc4n3)cc2)nc1)NC1COC1] | PA188 | 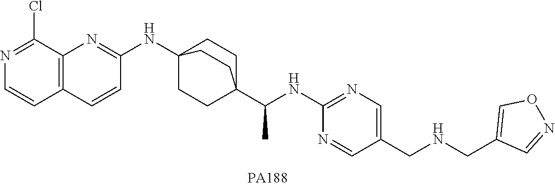 |
| PA034 | 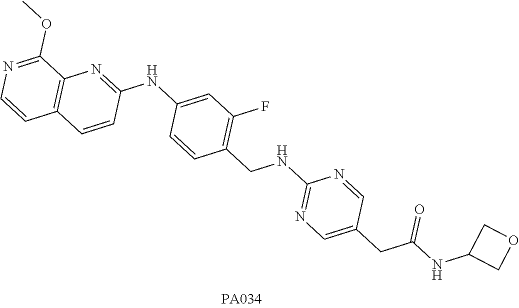 [SMILES: COc1nccc2ccc(Nc3ccc(CNc4ncc(CC(=O)NC5COC5)cn4)c(F)c3)nc12] | PA189 | 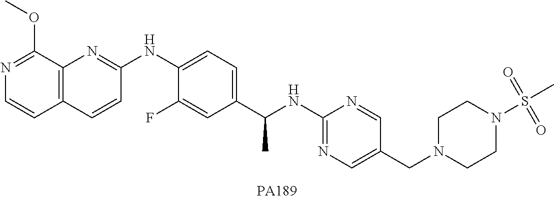 |
| PA038 | 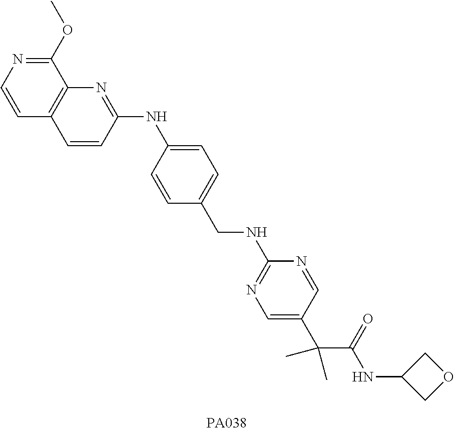 [SMILES: COc1nccc2ccc(Nc3ccc(CNc4ncc(C(C)(C)C(=O)NC5COC5)cn4)cc3)nc12] | PA190 | 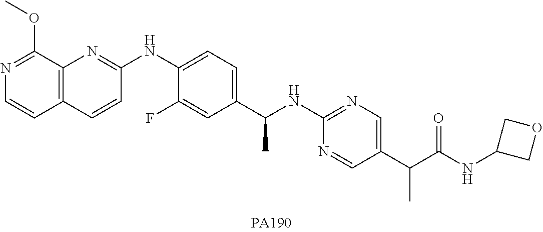 |
| PA039 | 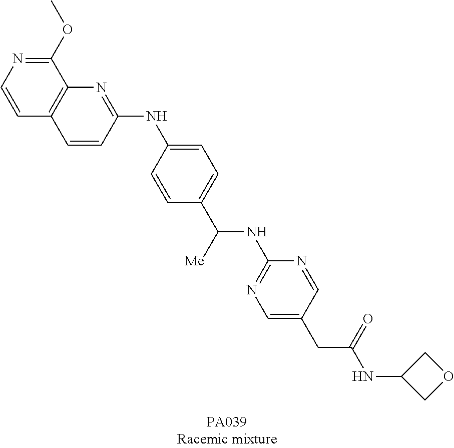 [SMILES: COc1nccc2ccc(Nc3ccc(C(C)Nc4ncc(CC(=O)NC5COC5)cn4)cc3)nc12] | PA191 | 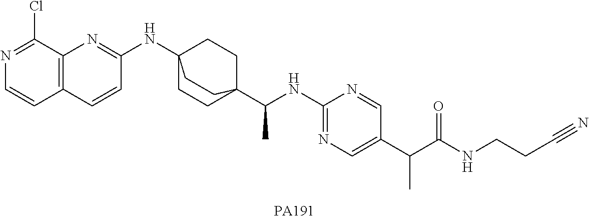 |
| PA041 | 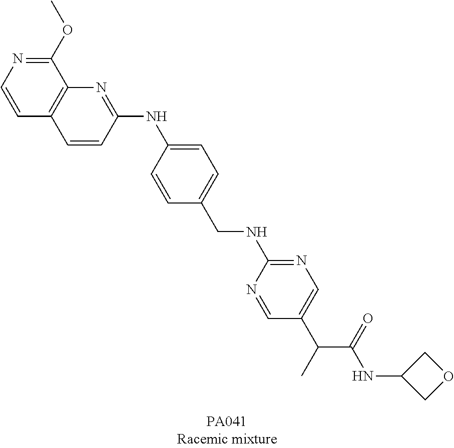 [SMILES: COc1nccc2ccc(Nc3ccc(CNc4ncc(C(C)C(=O)NC5COC5)cn4)cc3)nc12] | PA192 | 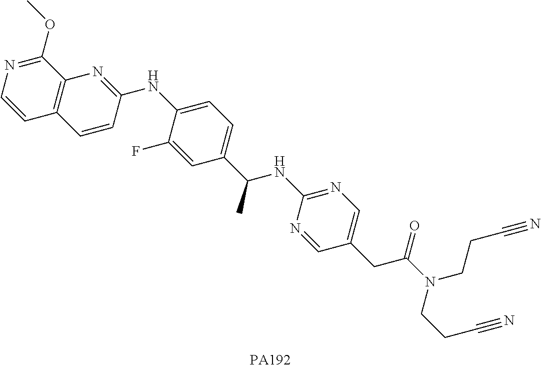 |
| PA042 | | PA193 | 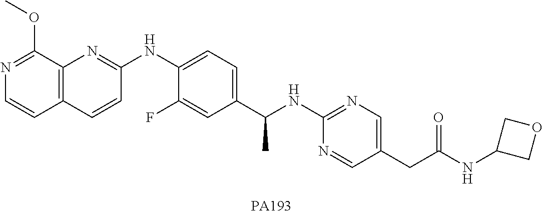 |
| PA043 | | PA194 | 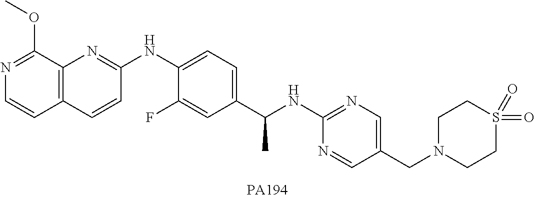 |
| PA044 | 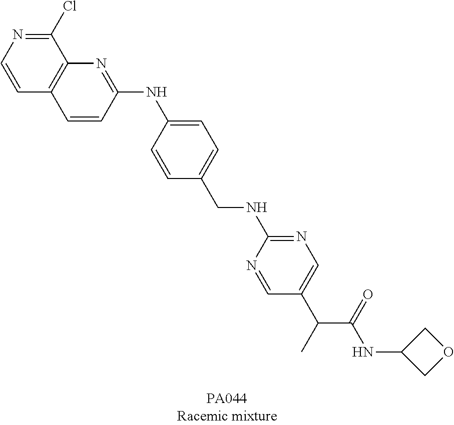 [SMILES: CC(C(=O)NC1COC1)c1cnc(NCc2ccc(Nc3ccc4ccnc(Cl)c4n3)cc2)nc1] | PA195 | 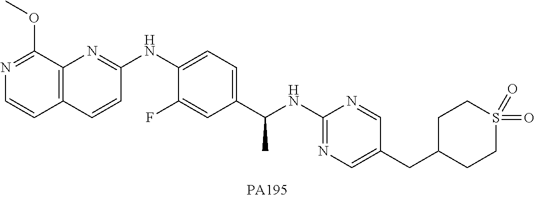 |
| PA045 | 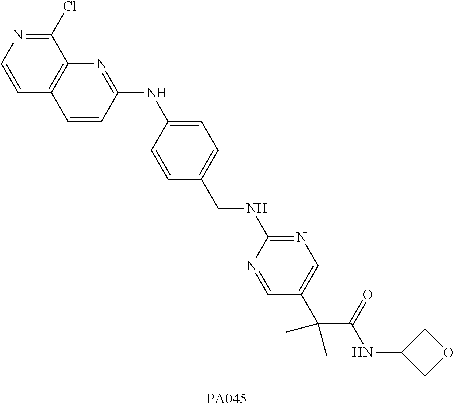 [SMILES: CC(C)(C(=O)NC1COC1)c1cnc(NCc2ccc(Nc3ccc4ccnc(Cl)c4n3)cc2)nc1] | PA196 | 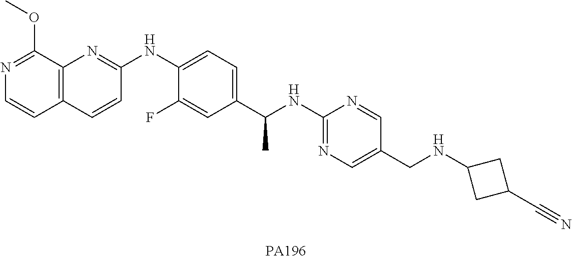 |
| PA046 | 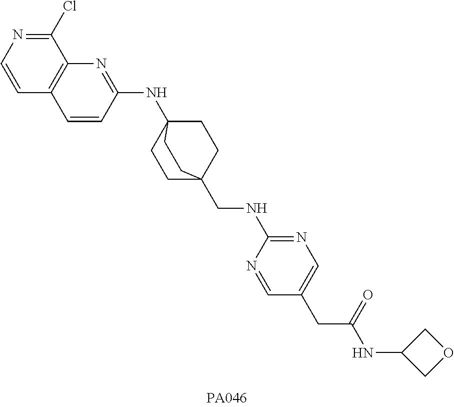 [SMILES: O=C(Cc1cnc(NCC23CCC(Nc4ccc5ccnc(Cl)c5n4)(CC2)CC3)nc1)NC1COC1] | PA197 | 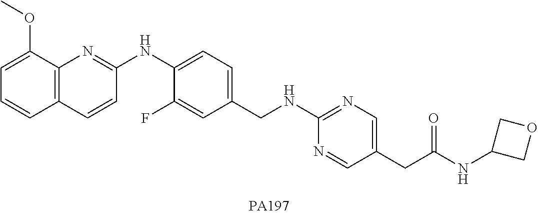 |
| PA048 | 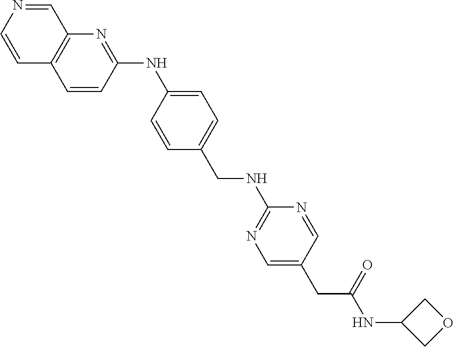 [SMILES: O=C(Cc1cnc(NCc2ccc(Nc3ccc4ccncc4n3)cc2)nc1)NC1COC1] | PA198 | 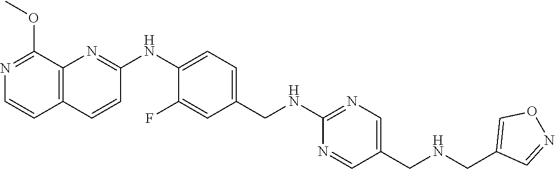 |
| | PA048 | | PA198 |
| PA051 | 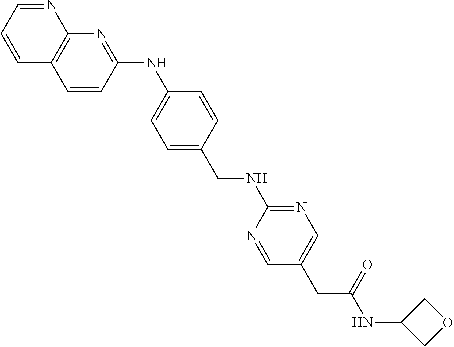 [SMILES: O=C(Cc1cnc(NCc2ccc(Nc3ccc4cccnc4n3)cc2)nc1)NC1COC1] | PA199 | 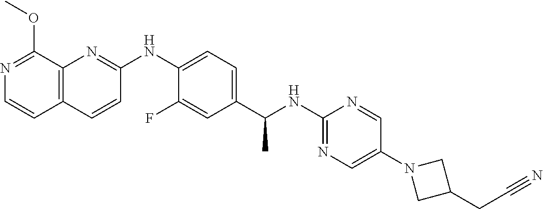 |
| | PA051 | | PA199 |
| PA52 | 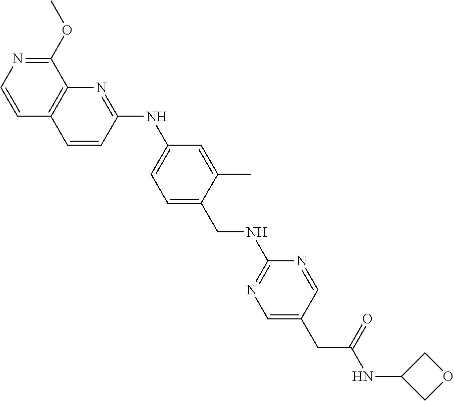 [SMILES: COc1nccc2ccc(Nc3ccc(CNc4ncc(CC(=O)NC5COC5)cn4)c(C)c3)nc12] | PA200 | 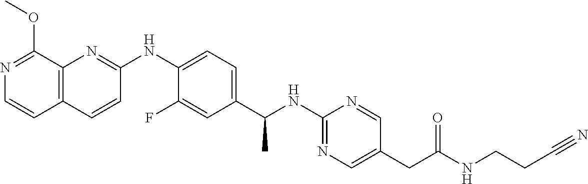 |
| | PA052 | | PA200 |
| PA053 | 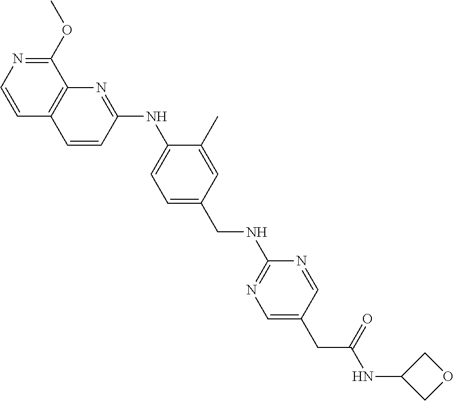 [SMILES: COc1nccc2ccc(Nc3ccc(CNc4ncc(CC(=O)NC5COC5)cn4)cc3C)nc12] | PA201 | 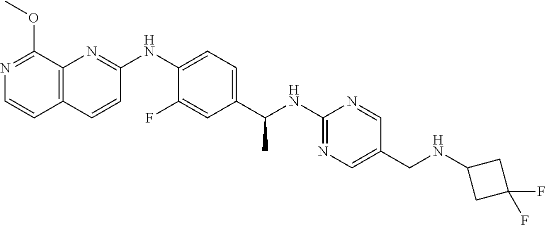 PA201 |
| | PA053 | | |
| PA054 | 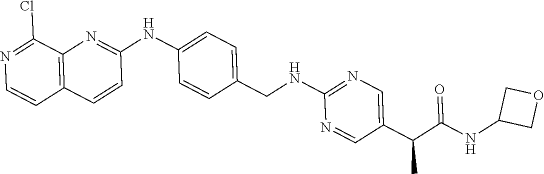 [SMILES: C[C@H](C(=O)NC1COC1)c1cnc(NCc2ccc(Nc3ccc4ccnc(Cl)c4n3)cc2)nc1] PA054 | PA202 | 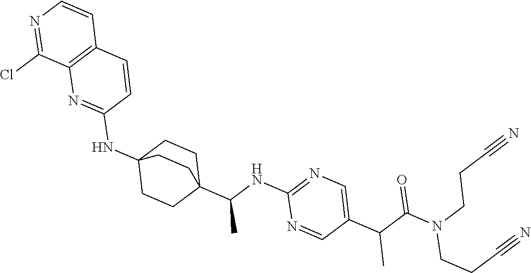 |
| | | | PA202 |
| PA055 | 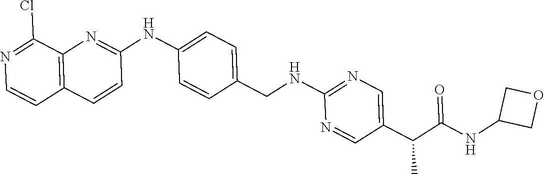 [SMILES: C[C@@H](C(=O)NC1COC1)c1cnc(NCc2ccc(Nc3ccc4ccnc(Cl)c4n3)cc2)nc1] | PA203 | 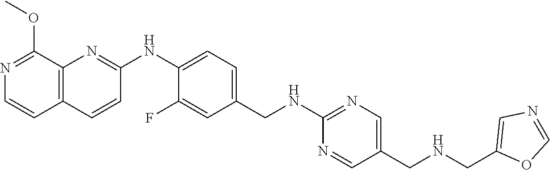 |
| | PA055 | | PA203 |
| PA056 | 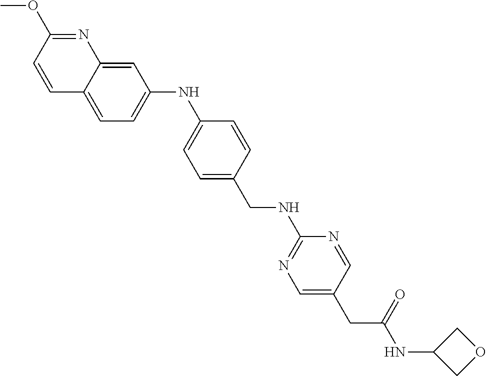 [SMILES: COc1ccc2ccc(Nc3ccc(CNc4ncc(CC(=O)NC5COC5)cn4)cc3)cc2n1] | PA204 | 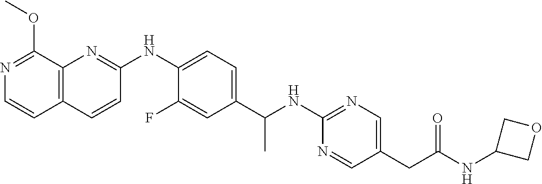 PA204 |
| | PA056 | | |
| PA058 | 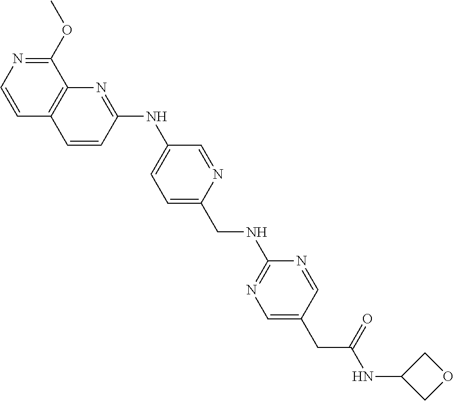 [SMILES: COc1nccc2ccc(Nc3ccc(CNc4ncc(CC(=O)NC5COC5)cn4)nc3)nc12] | PA205 | 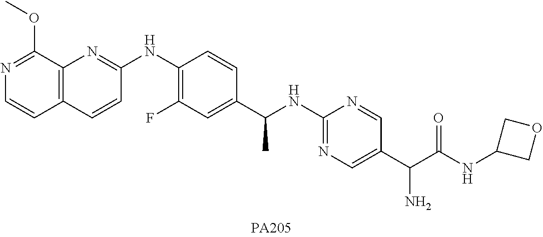 |
| | PA058 | | |
| PA061 | 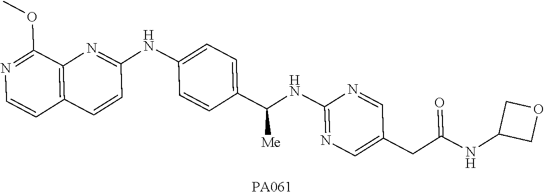 [SMILES: COc1nccc2ccc(Nc3ccc([C@H](C)Nc4ncc(CC(=O)NC5COC5)cn4)cc3)nc12] | PA206 | 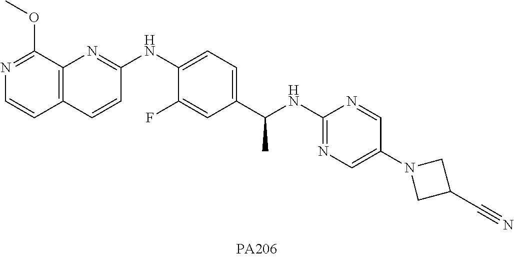 |
| PA062 | 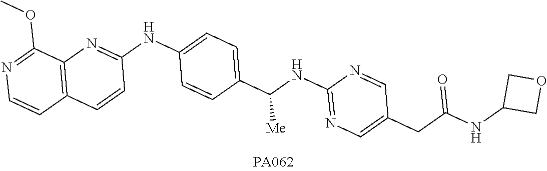 [SMILES: COc1nccc2ccc(Nc3ccc([C@@H](C)Nc4ncc(CC(=O)NC5COC5)cn4)cc3)nc12] | PA207 | 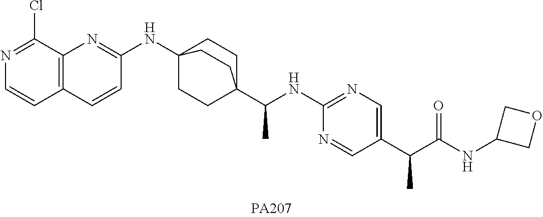 |
| PA063 | 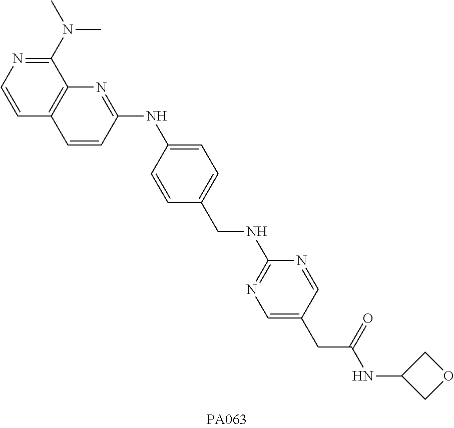 [SMILES: CN(C)c1nccc2ccc(Nc3ccc(CNc4ncc(CC(=O)NC5COC5)cn4)cc3)nc12] | PA208 | 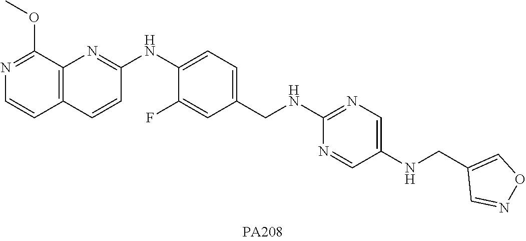 |
| PA064 | 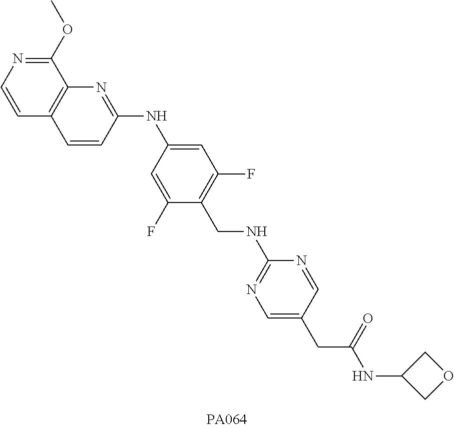 [SMILES: COc1nccc2ccc(Nc3cc(F)c(CNc4ncc(CC(=O)NC5COC5)cn4)c(F)c3)nc12] | PA209 | 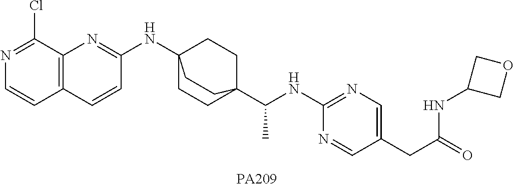 |
| PA066 | 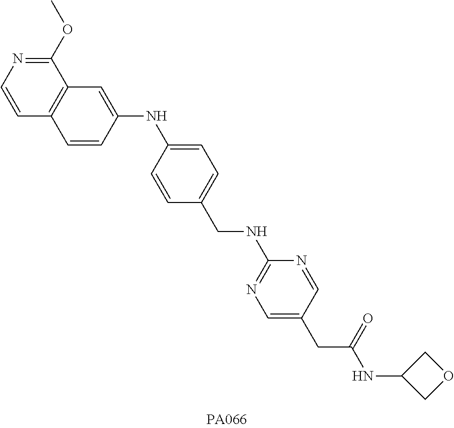 [SMILES: COc1nccc2ccc(Nc3ccc(CNc4ncc(CC(=O)NC5COC5)cn4)cc3)cc12] | PA210 | 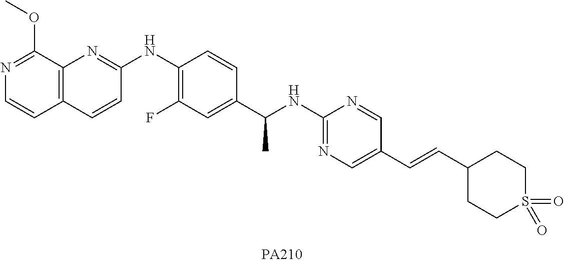 |
| PA068 | 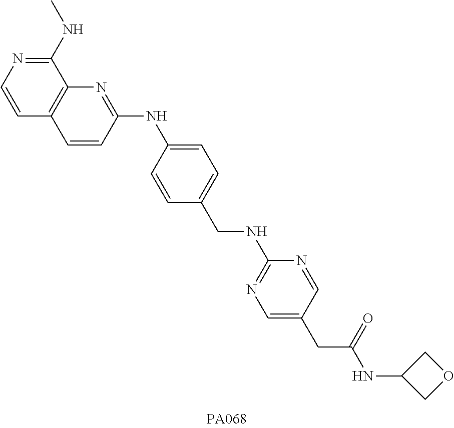 [SMILES: CNc1nccc2ccc(Nc3ccc(CNc4ncc(CC(=O)NC5COC5)cn4)cc3)nc12] | PA211 | 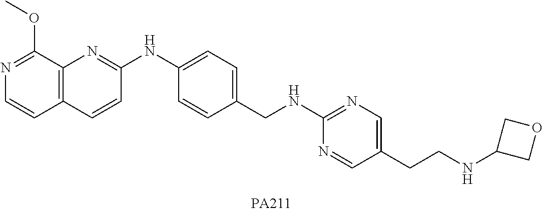 |
| PA069 | 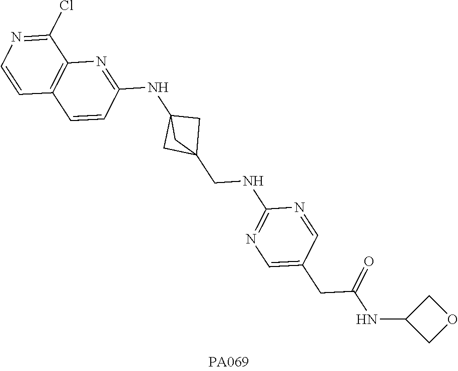 [SMILES: O=C(Cc1cnc(NCC23CC(Nc4ccc5ccnc(Cl)c5n4)(C2)C3)nc1)NC1COC1] | PA212 | 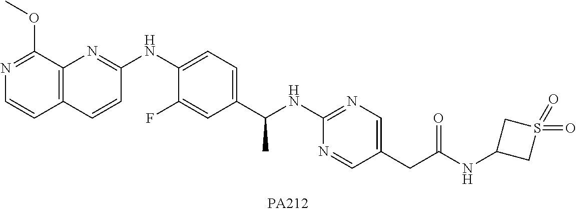 |
| PA070 | 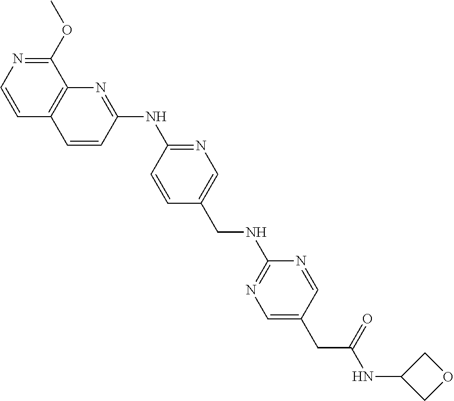 [SMILES: COc1nccc2ccc(Nc3ccc(CNc4ncc(CC(=O)NC5COC5)cn4)cn3)nc12] | PA213 | 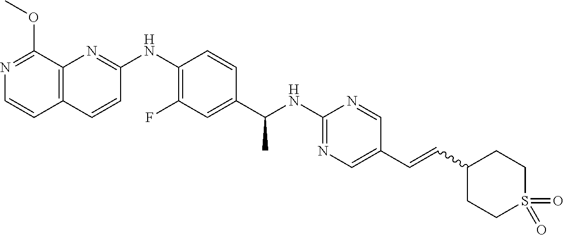 PA213 |
| | PA070 | | |
| PA072 | 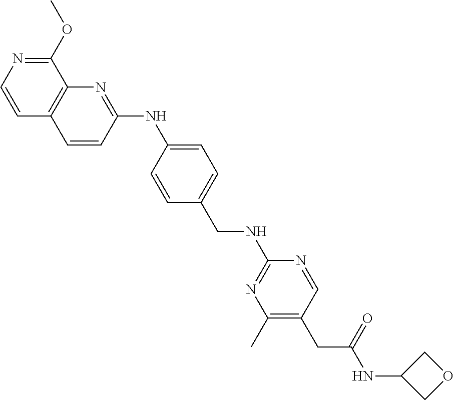 [SMILES: COc1nccc2ccc(Nc3ccc(CNc4ncc(CC(=O)NC5COC5)c(C)n4)cc3)nc12] | PA214 | 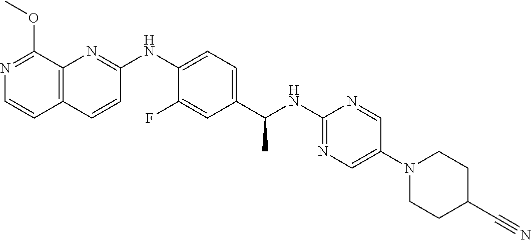 |
| | PA072 | | PA214 |
| PA073 | 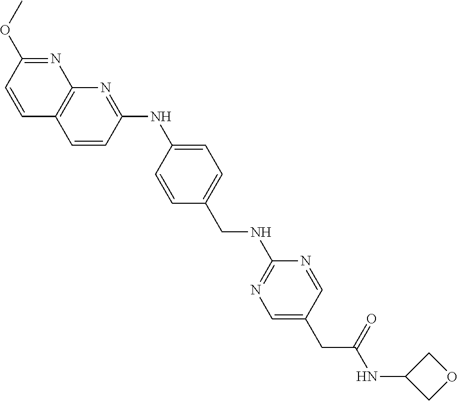 [SMILES: COc1ccc2ccc(Nc3ccc(CNc4ncc(CC(=O)NC5COC5)cn4)cc3)nc2n1] | PA215 | 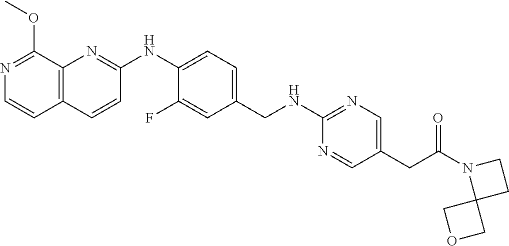 PA215 |
| | PA073 | | |
| PA074 | 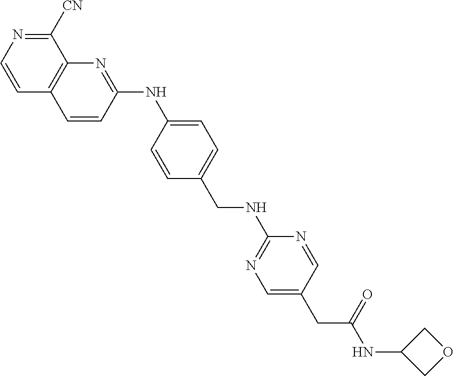 [SMILES: N#Cc1nccc2ccc(Nc3ccc(CNc4ncc(CC(=O)NC5COC5)cn4)cc3)nc12] | PA216 | 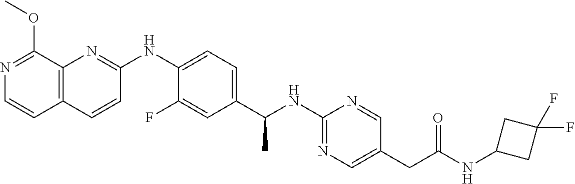 PA216 |
| | PA074 | | |
| PA075 | 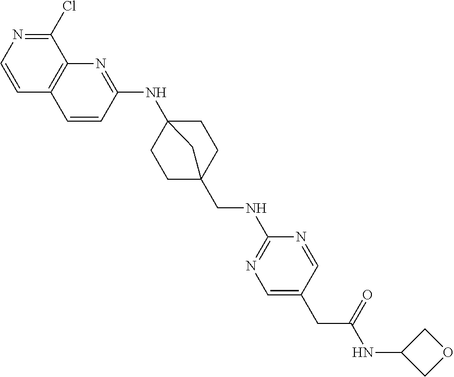 [SMILES: O=C(Cc1cnc(NCC23CCC(Nc4ccc5ccnc(Cl)c5n4)(CC2)C3)nc1)NC1COC1] | PA217 | 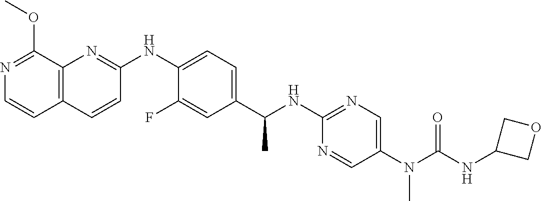 PA217 |
| | PA075 | | |
| PA076 | 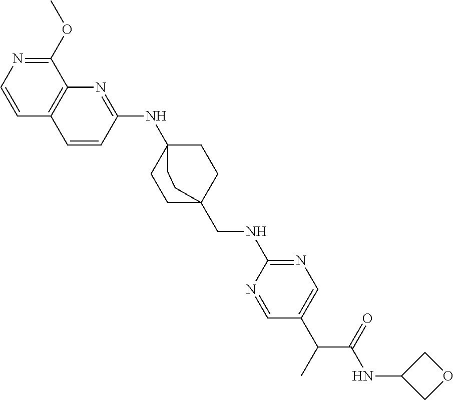 [SMILES: COc1nccc2ccc(NC34CCC(CNc5ncc(C(C)C(=O)NC6COC6)cn5)(CC3)CC4)nc12] | PA218 | 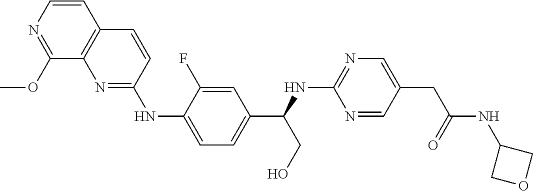 PA218 |
| | PA076 | | |
| PA077 | 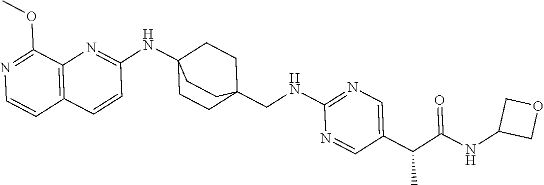 [SMILES: COc1nccc2ccc(NC34CCC(CNc5ncc([C@@H](C)C(=O)NC6COC6)cn5)(CC3)CC4)nc12] PA077 | PA219 | 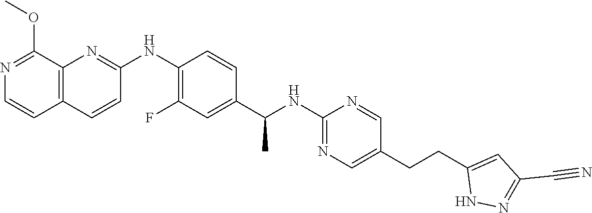 |
| | | | PA219 |
| PA078 | 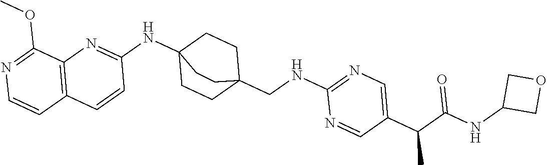 [SMILES: COc1nccc2ccc(NC34CCC(CNc5ncc([C@H](C)C(=O)NC6COC6)cn5)(CC3)CC4)nc12] | PA220 | 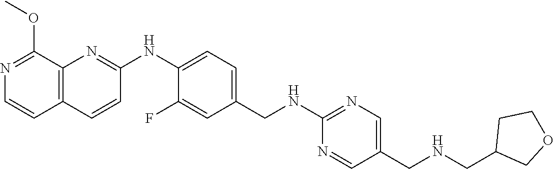 |
| | PA078 | | PA220 |
| PA079 | 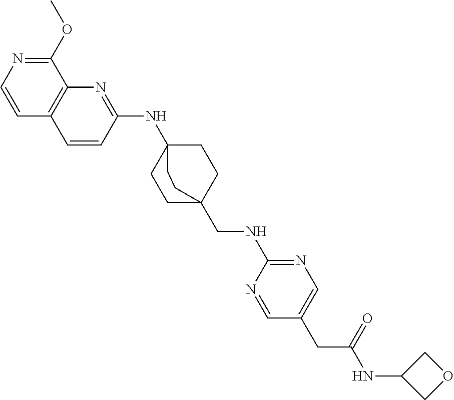 [SMILES: COc1nccc2ccc(NC34CCC(CNc5ncc(CC(=O)NC6COC6)cn5)(CC3)CC4)nc12] | PA221 | 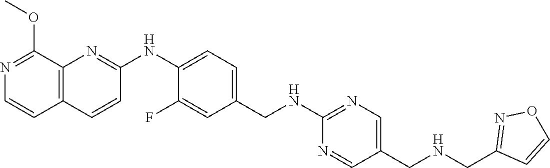 PA221 |
| | PA079 | | |
| PA081 | 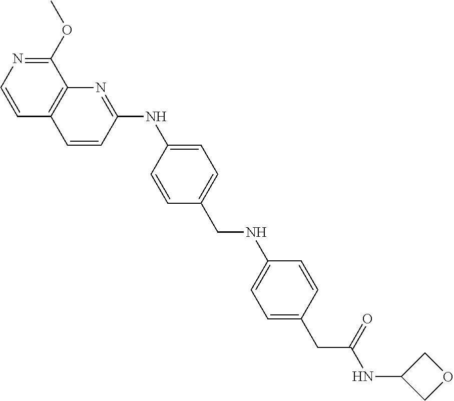 [SMILES: COc1nccc2ccc(Nc3ccc(CNc4ccc(CC(=O)NC5COC5)cc4)cc3)nc12] | PA222 | 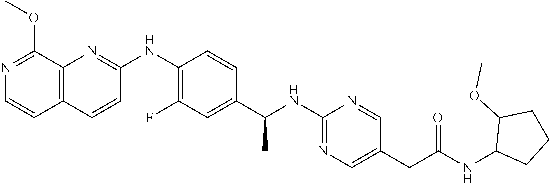 PA222 |
| | PA081 | | |
| PA082 | 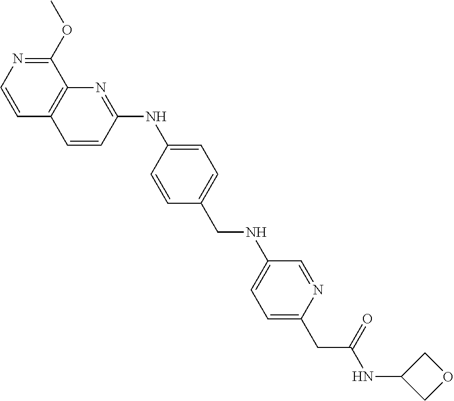 [SMILES: COc1nccc2ccc(Nc3ccc(CNc4ccc(CC(=O)NC5COC5)nc4)cc3)nc12] | PA223 | 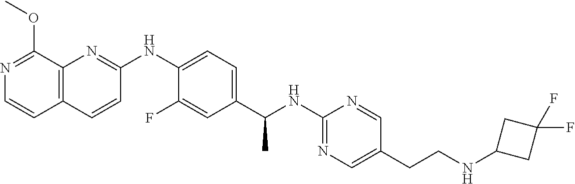 PA223 |
| | PA082 | | |
| PA085 | 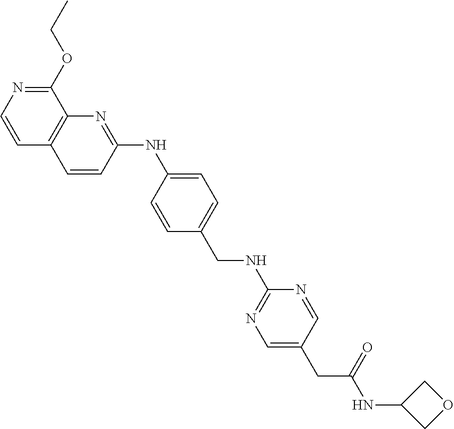 [SMILES: CCOc1nccc2ccc(Nc3ccc(CNc4ncc(CC(=O)NC5COC5)cn4)cc3)nc12] | PA224 | 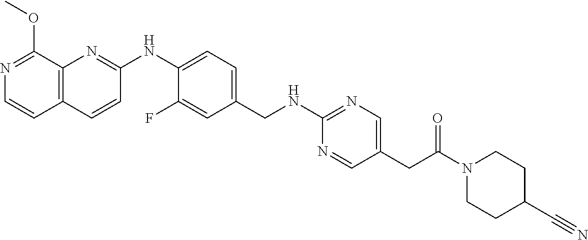 PA224 |
| | PA085 | | |
| PA089 | 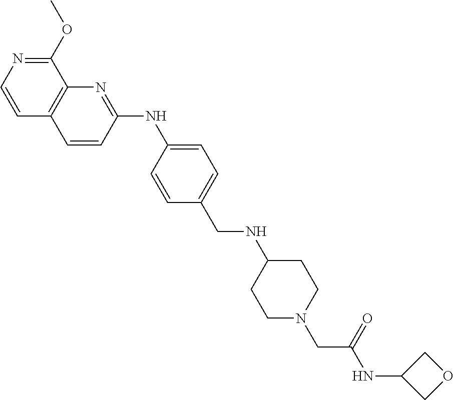 [SMILES: COc1nccc2ccc(Nc3ccc(CNC4CCN(CC(=O)NC5COC5)CC4)cc3)nc12] | PA225 | 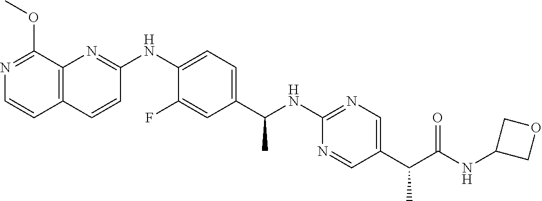 PA225 |
| | PA089 | | |
| PA106 | 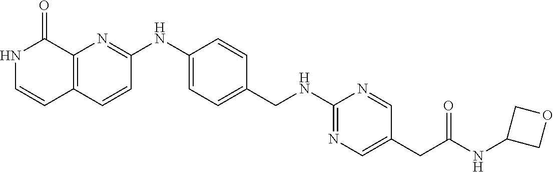 [SMILES: O=C(Cc1cnc(NCc2ccc(Nc3ccc4cc[nH]c(=O)c4n3)cc2)nc1)NC1COC1] PA106 | PA226 | 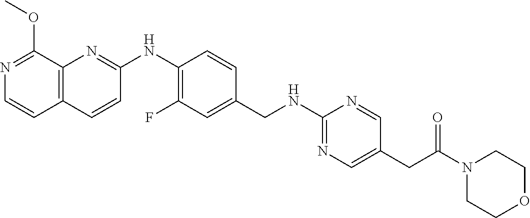 |
| | | | PA226 |
| PA110 | 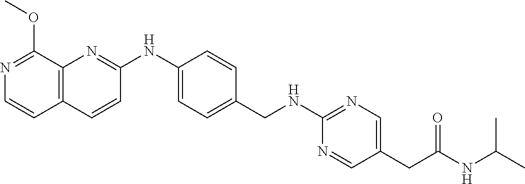 [SMILES: COc1nccc2ccc(Nc3ccc(CNc4ncc(CC(=O)NC(C)C)cn4)cc3)nc12] | PA227 | 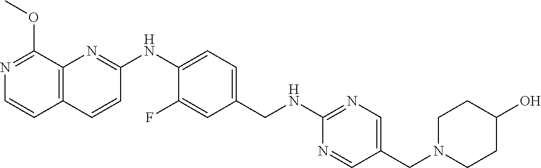 |
| | PA110 | | PA227 |
| PA112 | 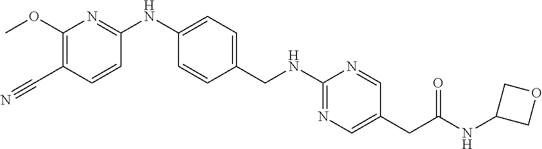 [SMILES: COc1nc(Nc2ccc(CNc3ncc(CC(=O)NC4COC4)cn3)cc2)ccc1C#N] PA112 | PA228 | 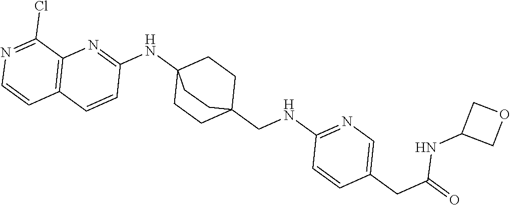 |
| | | | PA228 |
| PA113 | 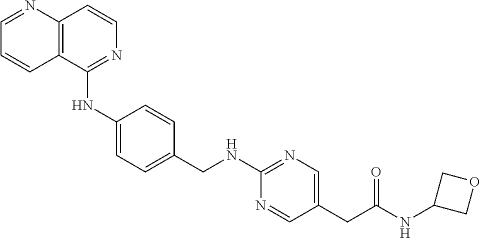 [SMILES: O=C(Cc1cnc(NCc2ccc(Nc3nccc4ncccc34)cc2)nc1)NC1COC1] | PA229 | 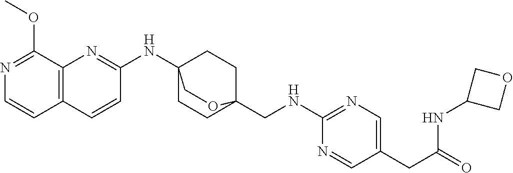 PA229 |
| | PA113 | | |
| PA122 | 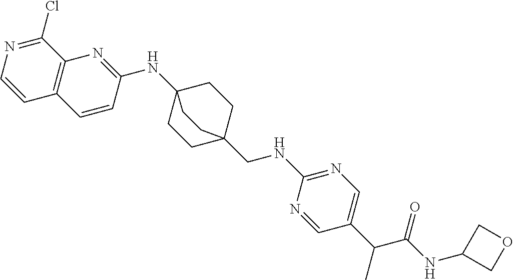 [SMILES: CC(C(=O)NC1COC1)c1cnc(NCC23CCC(Nc4ccc5ccnc(Cl)c5n4)(CC2)CC3)nc1] | PA230 | 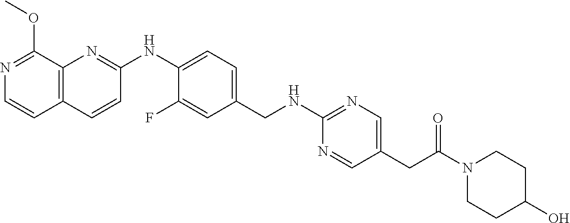 PA230 |
| | PA122 | | |
| PA123 | 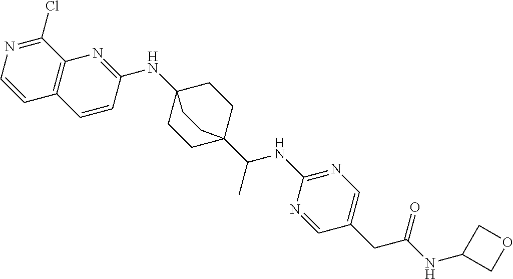 [SMILES: CC(Nc1ncc(CC(=O)NC2COC2)cn1)C12CCC(Nc3ccc4ccnc(Cl)c4n3)(CC1)CC2] | PA231 | 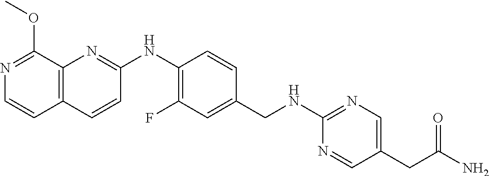 PA231 |
| | PA123 | | |
| PA124 | 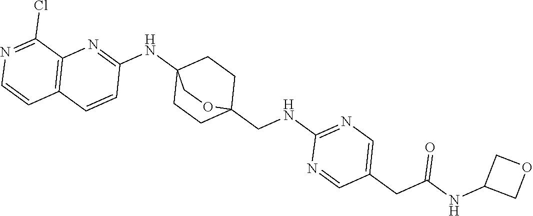 [SMILES: O=C(Cc1cnc(NCC23CCC(Nc4ccc5ccnc(Cl)c5n4)(CC2)CO3)nc1)NC1COC1] | PA232 | 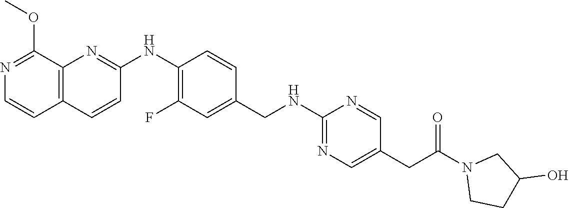 |
| | PA124 | | PA232 |
| PA151 | 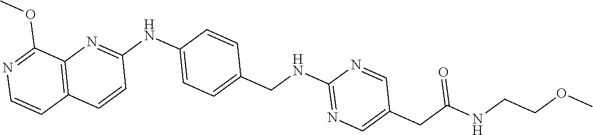 PA151 | PA233 | 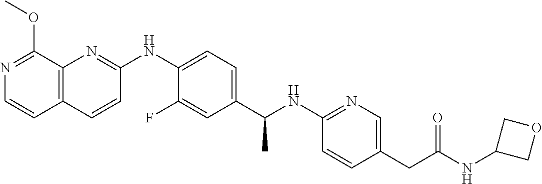 |
| | | | PA233 |
| PA152 | 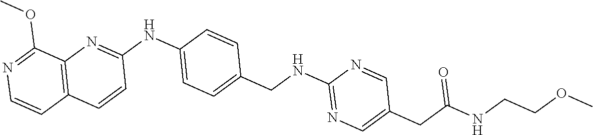 PA152 | PA234 | 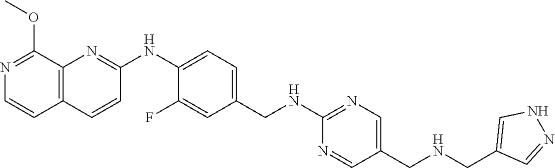 |
| | | | PA234 |
| PA153 | 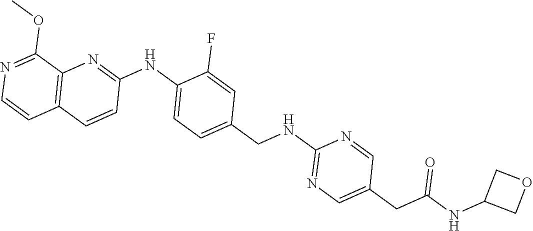 [SMILES: COc1nccc2ccc(Nc3ccc(CNc4ncc(CC(=O)NC5COC5)cn4)cc3F)nc12] | PA235 | 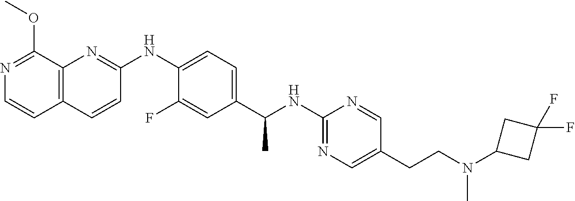 PA235 |
| | PA153 ZI | | |
| PA154 | 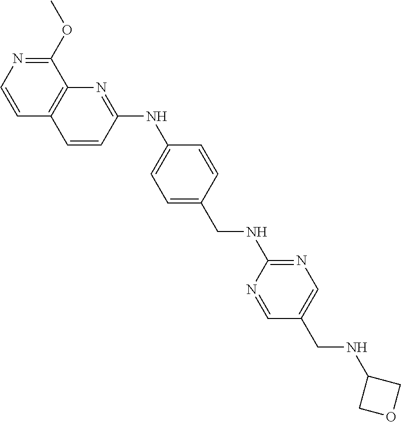 [SMILES: COc1nccc2ccc(Nc3ccc(CNc4ncc(CNC5COC5)cn4)cc3)nc12] | PA236 | 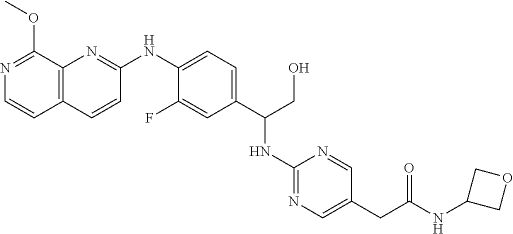 PA236 |
| | PA154 | | |
| PA155 | 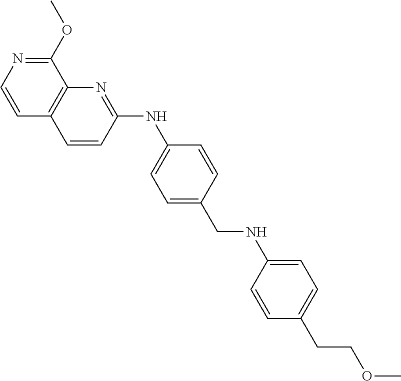 [SMILES: COCCc1ccc(NCc2ccc(Nc3ccc4ccnc(OC)c4n3)cc2)cc1] | PA237 | 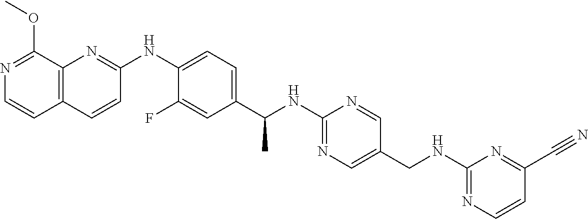 PA237 |
| | PA155 | | |
| PA156 | 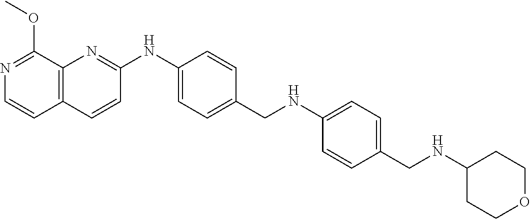 [SMILES: COc1nccc2ccc(Nc3ccc(CNc4ccc(CNC5CCOCC5)cc4)cc3)nc12] | PA238 | 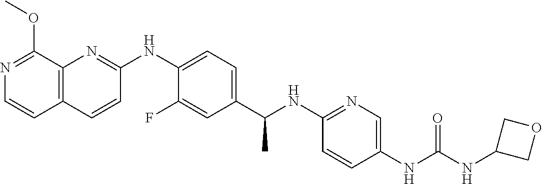 PA238 |
| | PA156 | | |
| PA157 | 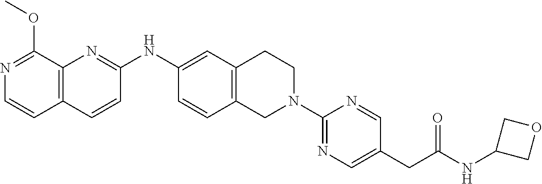 [SMILES: COc1nccc2ccc(Nc3ccc4c(c3)CCN(c3ncc(CC(=O)NC5COC5)cn3)C4)nc12] | PA239 | 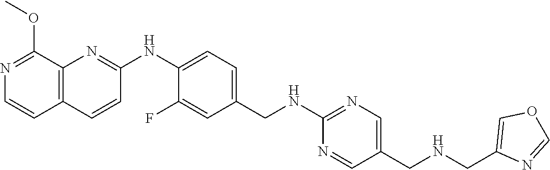 |
| | PA157 | | PA239 |
| PA158 | 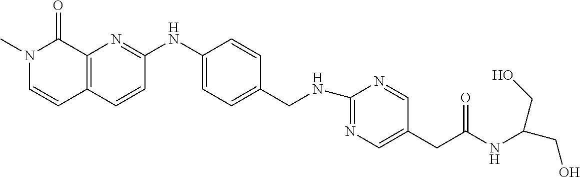 [SMILES: Cn1ccc2ccc(Nc3ccc(CNc4ncc(CC(=O)NC(CO)CO)cn4)cc3)nc2c1=O] | PA240 | 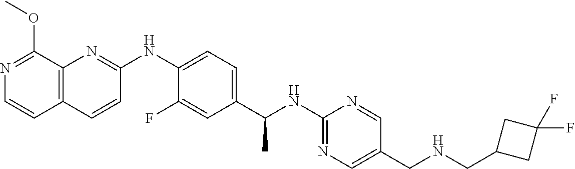 |
| | PA158 | | PA240 |
| PA159 | 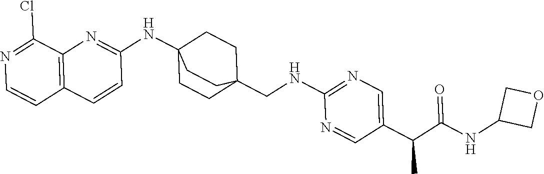 [SMILES: C[C@H](C(=O)NC1COC1)c1cnc(NCC23CCC(Nc4ccc5ccnc(Cl)c5n4)(CC2)CC3)nc1] PA159 | PA241 | 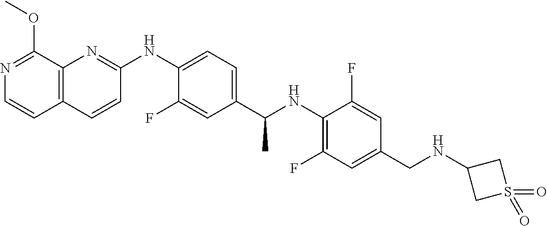 |
| | | | PA241 |
| PA160 | 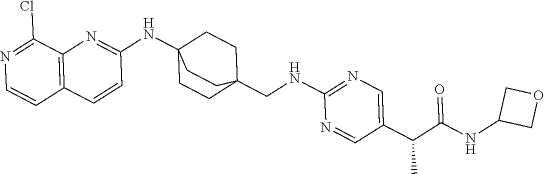 [SMILES: C[C@@H](C(=O)NC1COC1)c1cnc(NCC23CCC(Nc4ccc5ccnc(Cl)c5n4)(CC2)CC3)nc1] | PA242 | 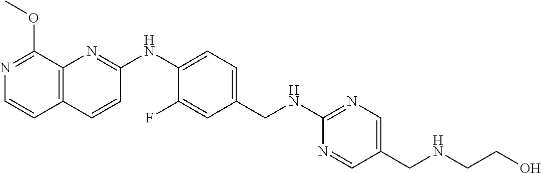 |
| | PA160 | | PA242 |
| PA161 | 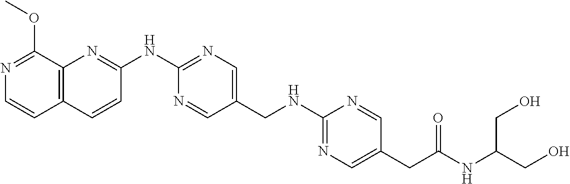 [SMILES: COc1nccc2ccc(Nc3ncc(CNc4ncc(CC(=O)NC(CO)CO)cn4)cn3)nc12] | PA243 | 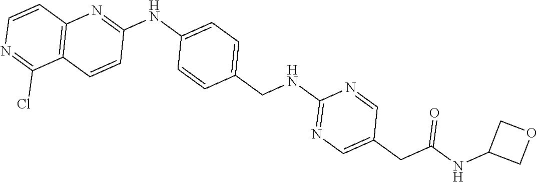 |
| | PA161 | | PA243 |
| PA162 | 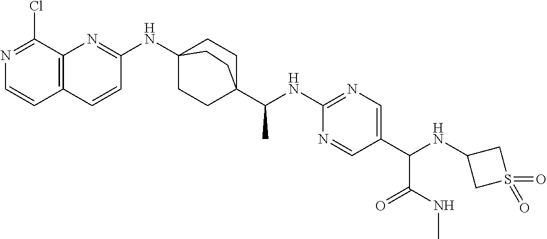 [SMILES: CNC(=O)C(NC1CS(=O)(=O)C1)c1cnc(N[C@@H](C)C23CCC(Nc4ccc5ccnc(Cl)c5n4)(CC2)CC3)nc1] | PA244 | 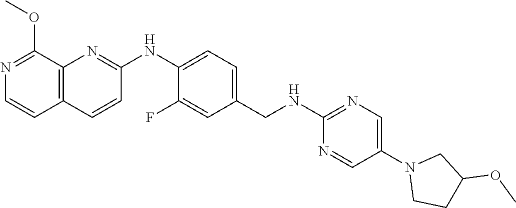 PA244 |
| | PA162 | | |
| PA163 | 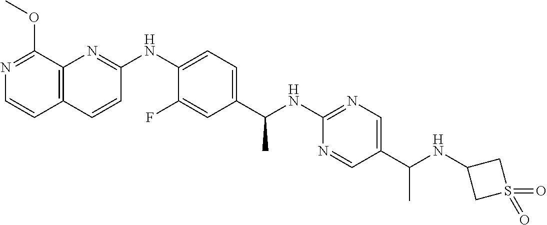 [SMILES: COc1nccc2ccc(Nc3ccc([C@H](C)Nc4ncc(C(C)NC5CS(=O)(=O)C5)cn4)cc3F)nc12] | PA245 | 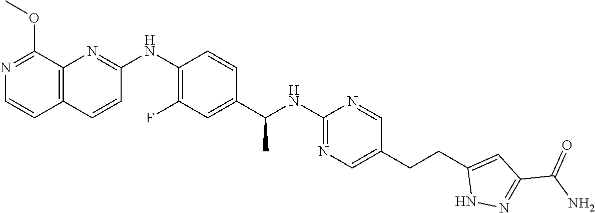 |
| | PA163 | | PA245 |
| PA164 | 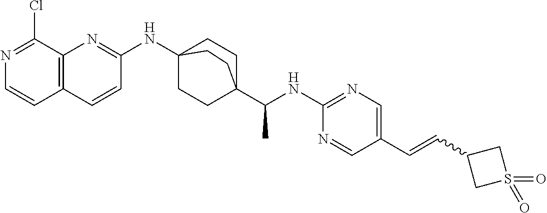 [SMILES: C[C@H](Nc1ncc(C=CC2CS(=O)(=O)C2)cn1)C12CCC(Nc3ccc4ccnc(Cl)c4n3)(CC1)CC2] | PA246 | 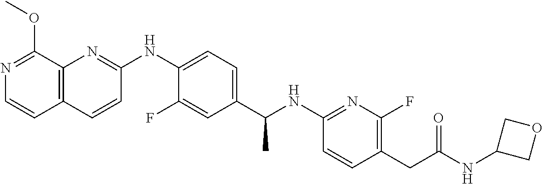 PA246 |
| | PA164 | | |
| PA165 | 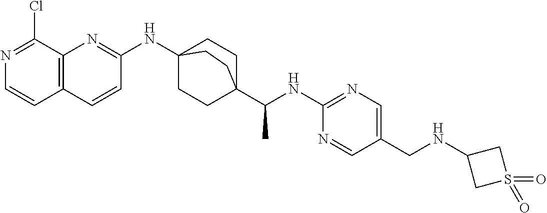 [SMILES: C[C@H](Nc1ncc(CNC2CS(=O)(=O)C2)cn1)C12CCC(Nc3ccc4ccnc(Cl)c4n3)(CC1)CC2] | PA247 | 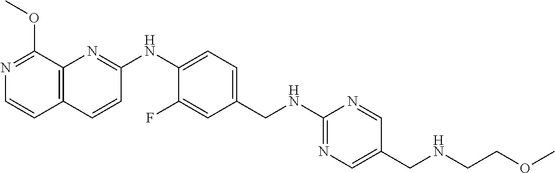 PA247 |
| | PA165 | | |
| PA166 | 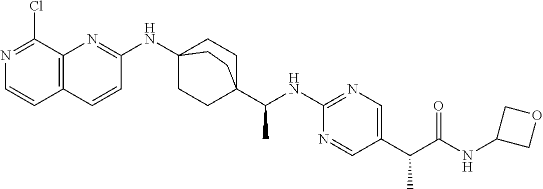 [SMILES: C[C@H](Nc1ncc([C@@H](C)C(=O)NC2COC2)cn1)C12CCC(Nc3ccc4ccnc(Cl)c4n3)(CC1)CC2] | PA248 | 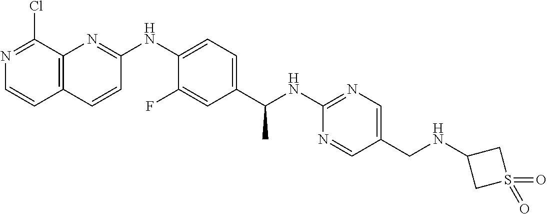 |
| | PA186 | | PA248 |
| | single compound | | |
| | Absolute configuration | | |
| | temporarily randomly assigned | | |
| PA167 | 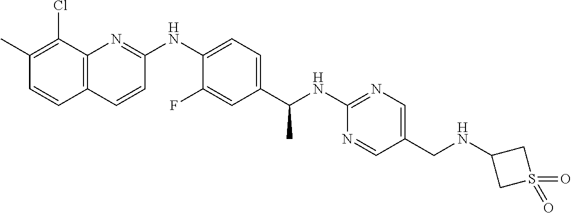 [SMILES: Cc1ccc2ccc(Nc3ccc([C@H](C)Nc4ncc(CNC5CS(=O)(=O)C5)cn4)cc3F)nc2c1Cl] | PA249 | 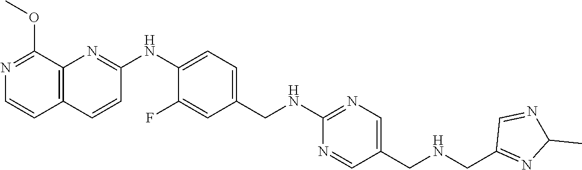 |
| | PA167 | | PA249 |
| PA168 | 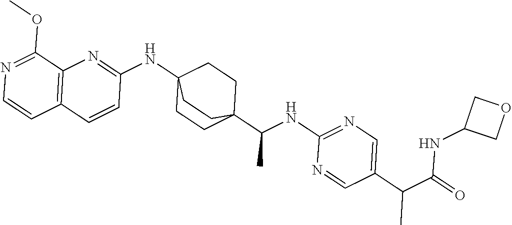 [SMILES: COc1nccc2ccc(NC34CCC([C@H](C)Nc5ncc(C(C)C(=O)NC6COC6)cn5)(CC3)CC4)nc12] | PA250 | 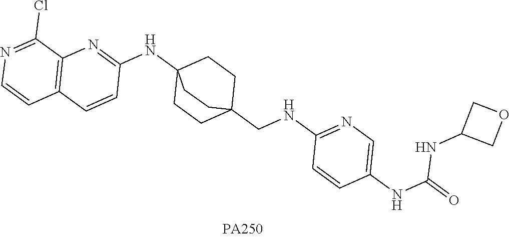 |
| | PA168 | | |
| PA169 | 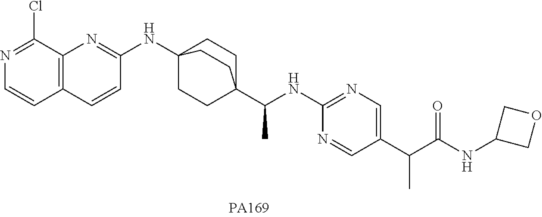 [SMILES: CC(C(=O)NC1COC1)c1cnc(N[C@@H](C)C23CCC(Nc4ccc5ccnc(Cl)c5n4)(CC2)CC3)nc1] | PA251 | 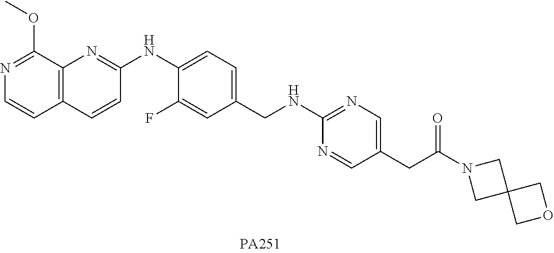 |
| PA170 | 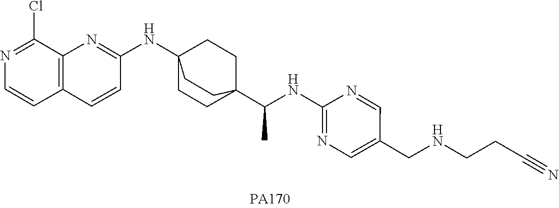 [SMILES: C[C@H](Nc1ncc(CNCCC#N)cn1)C12CCC(Nc3ccc4ccnc(Cl)c4n3)(CC1)CC2] | PA252 | 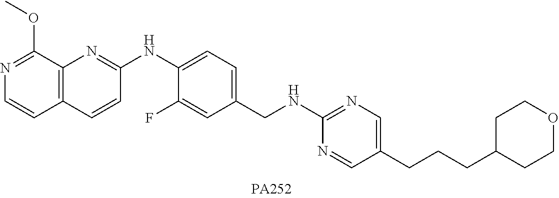 |
| PA171 | 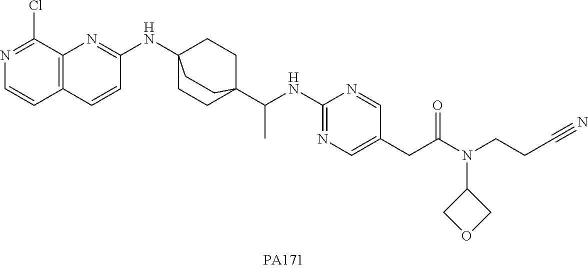 [SMILES: CC(Nc1ncc(CC(=O)N(CCC#N)C2COC2)cn1)C12CCC(Nc3ccc4ccnc(Cl)c4n3)(CC1)CC2] | PA253 | 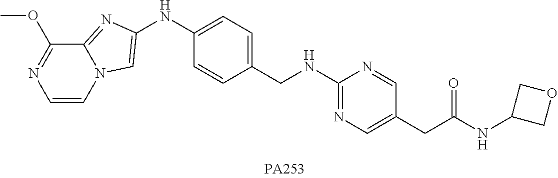 |
| PA172 | 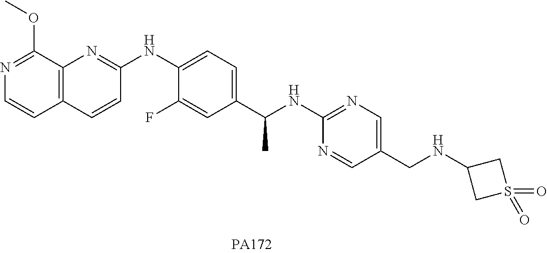 [SMILES: COc1nccc2ccc(Nc3ccc([C@H](C)Nc4ncc(CNC5CS(=O)(=O)C5)cn4)cc3F)nc12] | PA254 | 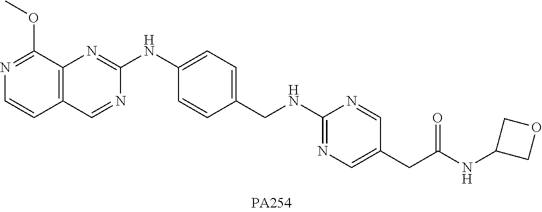 |
| PA173 | 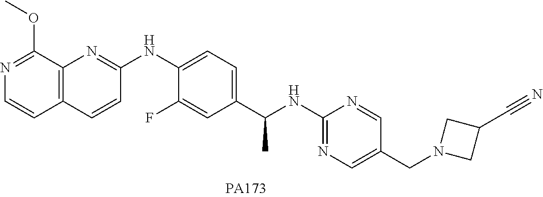 [SMILES: COc1nccc2ccc(Nc3ccc([C@H](C)Nc4ncc(CN5CC(C#N)C5)cn4)cc3F)nc12] | PA255 | 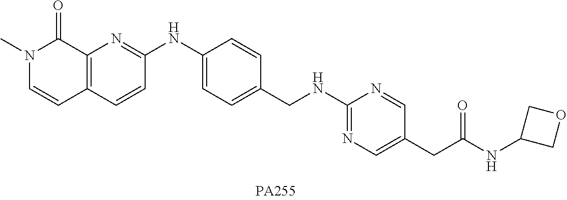 |
| PA174 | 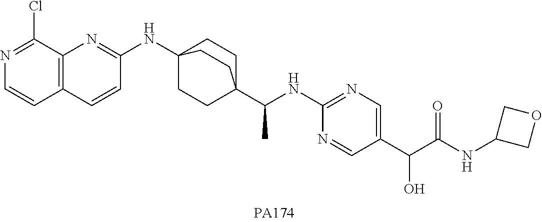 [SMILES: C[C@H](Nc1ncc(C(O)C(=O)NC2COC2)cn1)C12CCC(Nc3ccc4ccnc(Cl)c4n3)(CC1)CC2] | PA256 | 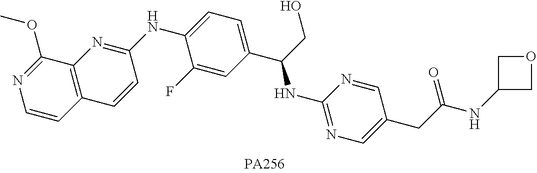 |
| PA175 | 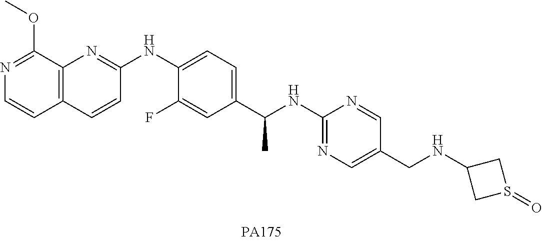 [SMILES: COc1nccc2ccc(Nc3ccc([C@H](C)Nc4ncc(CNC5CS(=O)C5)cn4)cc3F)nc12] | PA257 | 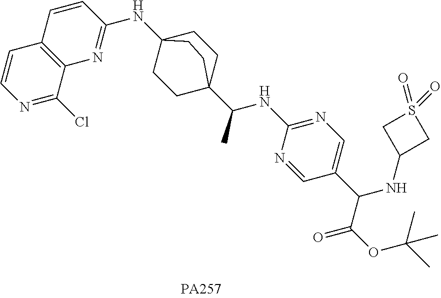 |
| PA176 | 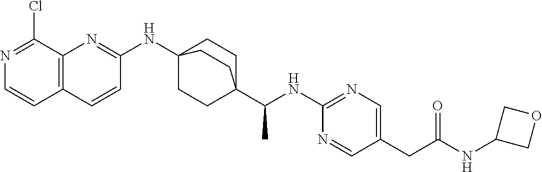 [SMILES: C[C@H](Nc1ncc(CC(=O)NC2COC2)cn1)C12CCC(Nc3ccc4ccnc(Cl)c4n3)(CC1)CC2] | PA258 | 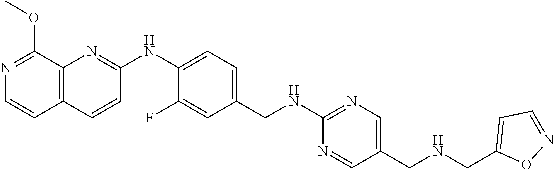 |
| | PA176 | | PA258 |
| PA177 | 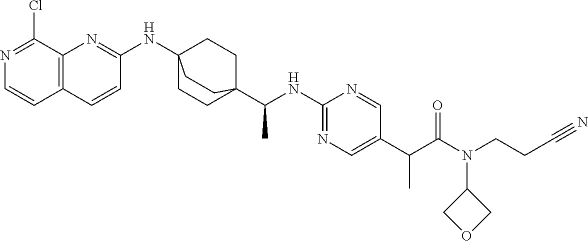 [SMILES: CC(C(=O)N(CCC#N)C1COC1)c1cnc(N[C@@H](C)C23CCC(Nc4ccc5ccnc(Cl)c5n4)(CC2)CC3)nc1] | PA259 | 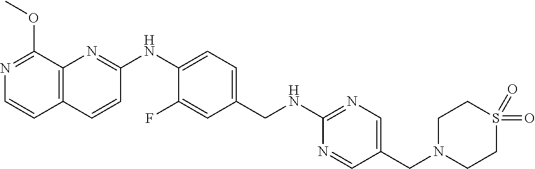 PA259 |
| | PA177 | | |
| PA179 | 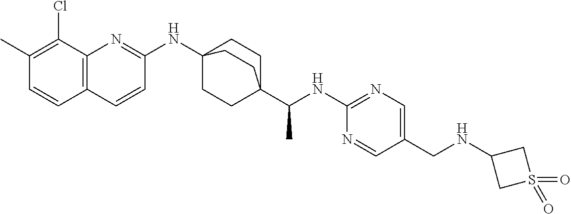 [SMILES: Cc1ccc2ccc(NC34CCC([C@H](C)Nc5ncc(CNC6CS(=O)(=O)C6)cn5)(CC3)CC4)nc2c1Cl] | PA260 | 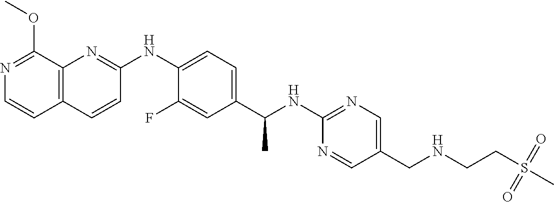 |
| | PA179 | | PA260 |
| PA180 | 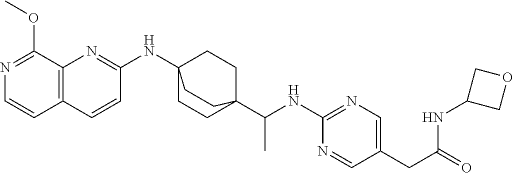 [SMILES: COc1nccc2ccc(NC34CCC(C(C)Nc5ncc(CC(=O)NC6COC6)cn5)(CC3)CC4)nc12] PA180 | PA261 | 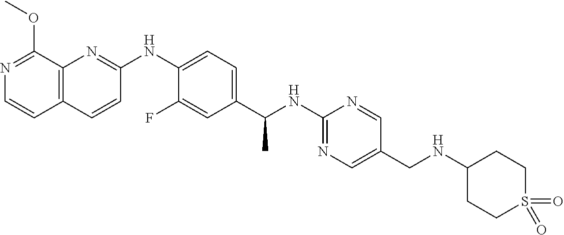 |
| | HN | | PA261 |
| PA181 | 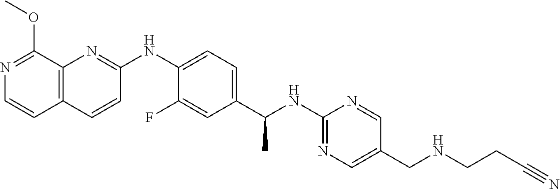 [SMILES: COc1nccc2ccc(Nc3ccc([C@H](C)Nc4ncc(CNCCC#N)cn4)cc3F)nc12] | PA262 | 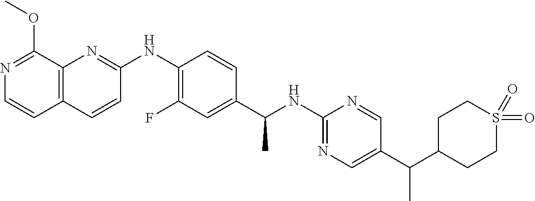 |
| | PA181 | | PA262 |
| PA182 | 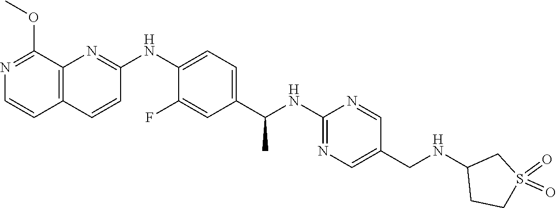 [SMILES: COc1nccc2ccc(Nc3ccc([C@H](C)Nc4ncc(CNC5CCS(=O)(=O)C5)cn4)cc3F)nc12] | PA263 | 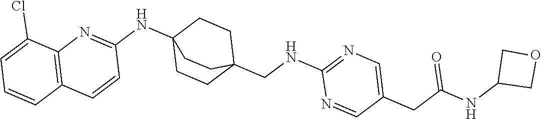 PA263 |
| | PA182 | | |
| PA183 | 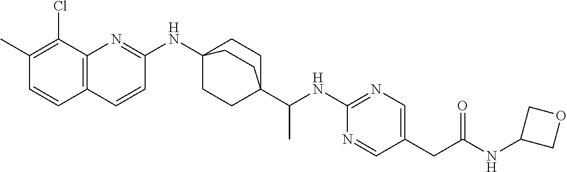 [SMILES: Cc1ccc2ccc(NC34CCC(C(C)Nc5ncc(CC(=O)NC6COC6)cn5)(CC3)CC4)nc2c1Cl] PA183 | PA264 | 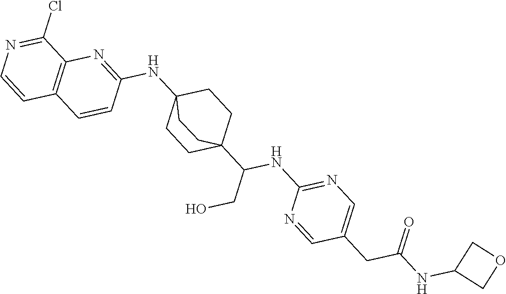 |
| | | | PA264 |
| PA184 | 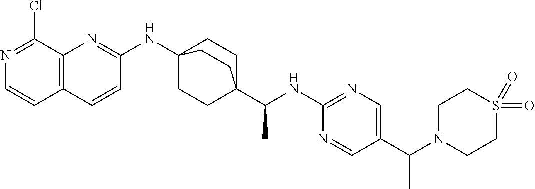 [SMILES: CC(c1cnc(N[C@@H](C)C23CCC(Nc4ccc5ccnc(Cl)c5n4)(CC2)CC3)nc1)N1CCS(=O)(=O)CC1] PA184 | PA265 | 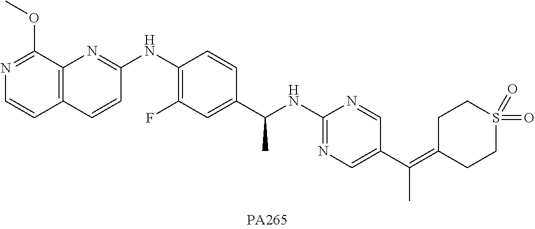 |
| PA266 | 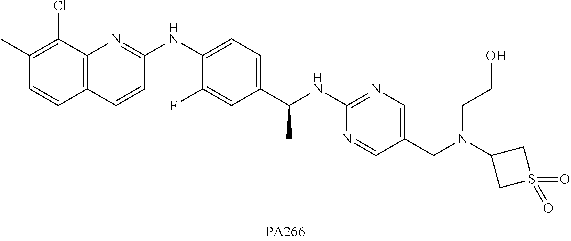 [SMILES: Cc1ccc2ccc(Nc3ccc([C@H](C)Nc4ncc(CN(CCO)C5CS(=O)(=O)C5)cn4)cc3F)nc2c1Cl] | PA267 | 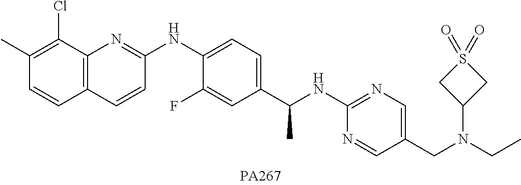 |
| PA268 | 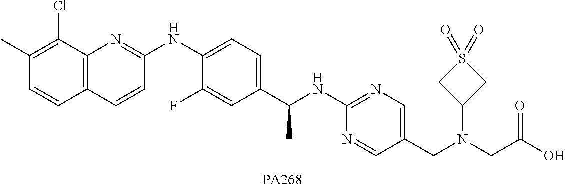 [SMILES: Cc1ccc2ccc(Nc3ccc([C@H](C)Nc4ncc(CN(CC(=O)O)C5CS(=O)(=O)C5)cn4)cc3F)nc2c1Cl] | PA269 | 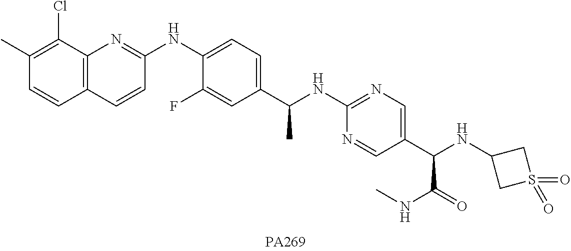 |
| PA270 | 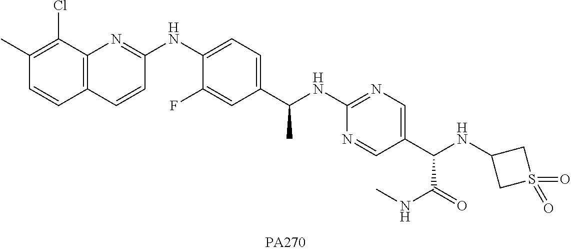 [SMILES: CNC(=O)[C@@H](NC1CS(=O)(=O)C1)c1cnc(N[C@@H](C)c2ccc(Nc3ccc4ccc(C)c(Cl)c4n3)c(F)c2)nc1] | PA271 | 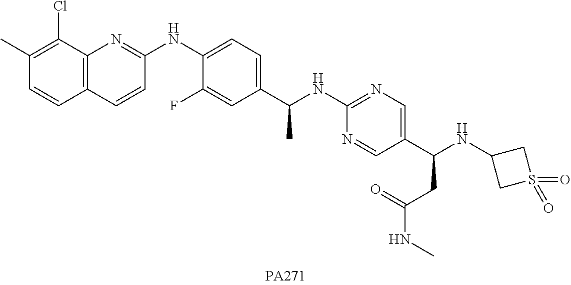 |
| PA272 | 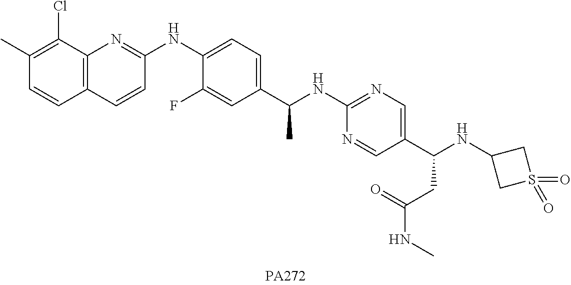 [SMILES: CNC(=O)C[C@@H](NC1CS(=O)(=O)C1)c1cnc(N[C@@H](C)c2ccc(Nc3ccc4ccc(C)c(Cl)c4n3)c(F)c2)nc1] | PA273 | 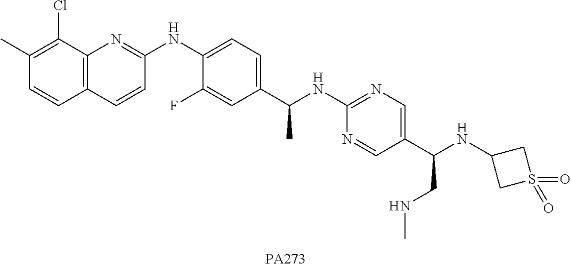 |
| PA274 | 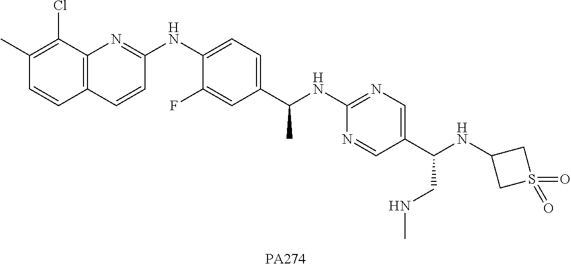 [SMILES: CNC[C@@H](NC1CS(=O)(=O)C1)c1cnc(N[C@@H](C)c2ccc(Nc3ccc4ccc(C)c(Cl)c4n3)c(F)c2)nc1] | PA275 | 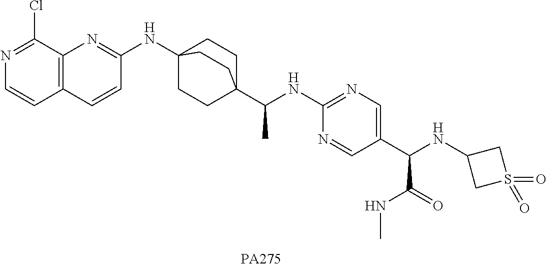 |
| PA276 | 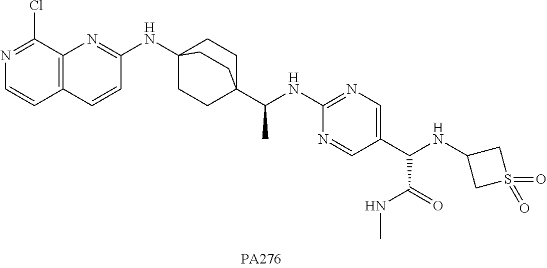 | PA277 | 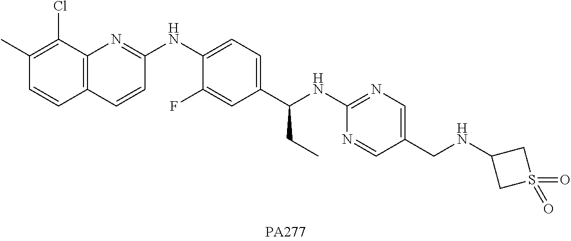 |
| PA278 | 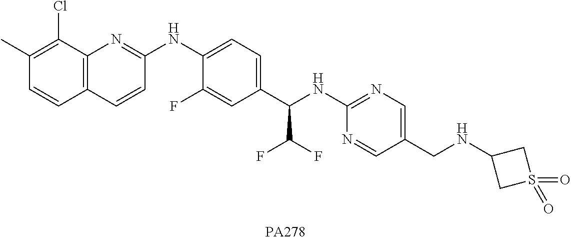 [SMILES: Cc1ccc2ccc(Nc3ccc([C@@H](Nc4ncc(CNC5CS(=O)(=O)C5)cn4)C(F)F)cc3F)nc2c1Cl] | PA279 | 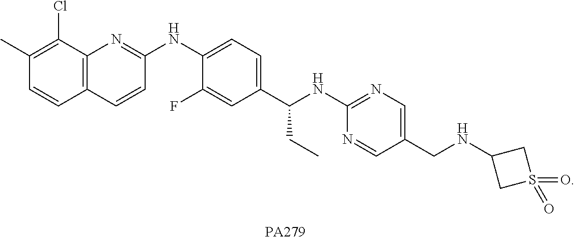 |

10. A pharmaceutical composition, characterized in that the pharmaceutical composition comprises a therapeutically effective amount of one or more of the compound according to claim 1, a pharmaceutically acceptable salt thereof, a racemate, an optical isomer, a stereoisomer, and a tautomer thereof, and one or more pharmaceutically acceptable carriers, excipients, adjuvants, excipients, and/or diluents.

11. A use of the compound according to claim 1, a racemate, an optical isomer monomer and mixtures thereof, or a pharmaceutically acceptable salt thereof in the preparation of a drug for the treatment or prevention of diseases associated with mutations, and overexpression of PRC2 complexes, monomers, or combinations thereof, or H3K127 methylation level dysregulation.

12. The use according to claim 11, characterized in that the disease is selected from the group consisting of: tumors and autoimmune diseases.

13. The use according to claim 11, characterized in that the disease is selected from the group consisting of: lymphoma, malignant hematopathy, sarcoma, prostate cancer, breast cancer, kidney cancer, urothelial cancer, gastric cancer, ovarian cancer, endometrial cancer, cervical cancer, lung cancer, liver cancer, pancreatic cancer, colon cancer, head and neck cancer, brain tumor, melanoma, mesothelioma, gastrointestinal stromal tumor, psoriasis, and lupus erythematosus.

14. The use according to claim 11, characterized in that the disease is selected from the group consisting of: non-Hodgkin lymphoma, follicular lymphoma, mantle cell lymphoma, peripheral T-cell lymphoma, Burkitt lymphoma, Hodgkin lymphoma, chronic lymphocytic leukemia, acute and chronic myeloid leukemia, acute and chronic lymphocytic leukemia, multiple myeloma, myelodysplastic syndrome, epithelioid sarcoma, rhabdomyosarcoma, liposarcoma, prostate cancer, breast cancer, kidney cancer, bladder cancer, upper urinary tract epithelial cancer, gastric cancer, ovarian cancer, endometrial cancer, cervical cancer, lung cancer, liver cancer, pancreatic cancer, colon cancer, head and neck cancer, medulloblastoma, glioma, schwannoma, melanoma, mesothelioma, gastrointestinal stromal tumor, psoriasis and lupus erythematosus.
